(19) United States
(12) Patent Application Publication
(10) Pub. No.: US 2026/0176300 A1
(43) Pub. Date: Jun. 25, 2026

(54) N-ARYLPYRAZOLE NOD2 AGONISTS AS PROMOTERS OF IMMUNE CHECKPOINT INHIBITOR THERAPY

(71) Applicants: THE SCRIPPS RESEARCH INSTITUTE, La Jolla, CA (US); THE ROCKEFELLER UNIVERSITY, New York, NY (US)

(72) Inventors: Howard HANG, Del Mar, CA (US); Taku TSUKIDATE, New York, NY (US); Matthew GRIFFIN, Irvine, CA (US)

(21) Appl. No.: 19/127,211

(22) PCT Filed: Nov. 6, 2023
(86) PCT No.: PCT/US23/78780

§ 371 Date: May 5, 2025

Related U.S. Application Data
(60) Provisional application No. 63/423,545, filed on Nov. 8, 2022.

(51) Int. Cl. C07K 5/062; A61K 38/00; A61K 45/06; A61P 37/04

(52) U.S. Cl. CPC C07K 5/06052; A61K 45/06; A61P 37/04; A61K 38/00

(57) ABSTRACT

The disclosure provides the development of enantiomer-specific 7V-arylpyrazole dipeptides as novel N0D2 agonists which are effective at promoting immune checkpoint inhibitor therapy requiring N0D2 for activity. Given the significant functions of N0D2 in innate and adaptive immunity, these novel agonists afford new therapeutic compounds for a variety of NOD2-responsive diseases.

### CROSS REFERENCE TO RELATED APPLICATION

This application claims priority to U.S. provisional patent application No. 63/423,545, which was filed on Nov. 8, 2022, and which is hereby incorporated by reference in its entirety.

### GOVERNMENT SUPPORT

This invention was made with government support under R01CA245292 awarded by the National Institutes of Health. The government has certain rights in the invention.

### FIELD OF THE INVENTION

The supplication relates to the development of enantiomer-specific N-arylpyrazole dipeptides as novel NOD2 agonists which are effective at promoting immune checkpoint inhibitor therapy requiring NOD2 for activity. Given the significant functions of NOD2 in innate and adaptive immunity, these novel agonists afford new therapeutic compounds for a variety of NOD2-responsive diseases.

### BACKGROUND OF THE INVENTION

Pattern recognition receptors (PRRs) provide important sensors for host recognition of microbes as well as environmental and cellular stresses to initiate innate and adaptive immunity responses.¹ These immune sensors have therefore emerged as excellent therapeutic targets for modulation of infection and inflammatory diseases in animals and humans. For example, the discovery of toll-like receptors (TLRs) has been transformative for understanding elucidating mechanisms of innate immunity and enabled the development several TLR-targeted therapeutics in clinical trials.² Beyond these PRRs, nucleotide-binding domain, leucine-rich repeat receptors (NLRs) also encode intracellular sensors of microbial ligands and stress.³ Amongst the NLRs, the founding members of this protein superfamily, nucleotide-binding and oligomerization domain (NOD) proteins 1 and 2 have been recognized key protein targets for immune activation.^{4,5} Notably, one of the key active components of the bacterial cell wall in Freund's adjuvant,⁶ muramyl dipeptide (MDP) is the minimal ligand required for the activation of NOD2 and downstream pro-inflammatory NF-κB signaling⁷⁻⁹. In contrast, NOD1 recognizes γ-d-glutamyl-meso-diaminopimelic acid (iE-DAP) an alternative peptidoglycan fragment of bacterial cell walls.^{10, 11} These two intracellular NLRs serve as important intracellular sensors of peptidoglycan fragments that is conserved in most bacterial species and crucial for host defense against diverse pathogens, as Nod1 and Nod2-deficient mice are more susceptible to bacterial and viral infections.¹² In addition to pathogens, these immune sensors of peptidoglycan also play key roles for detection of microbiota and maintenance of mucosal barriers. Notably, loss-of-function alleles of NOD2 are associated with compromised intestinal barrier function, microbiota dysbiosis and are major risk factors for inflammatory bowel disease (IBD) such as Crohn's disease.¹² Alternatively, gain-of-function NOD2 mutants are implicated in auto-inflammatory disease such as Blau syndrome.¹² Interestingly, NOD1 and NOD2 have also been implicated in sensing endoplasmic reticulum (ER) stress,¹³ potentially through binding of sphingosine-1-phosphate,¹⁴ and are also associated with inflammatory disorders such as obesity and neurodegeneration¹². Furthermore, NOD2 expression in hypothalamic inhibitory neurons has been shown to regulate appetite and body temperature in mice, which suggests additional roles for NOD2 in metabolism and behavior.¹⁵ These intracellular NLRs are therefore at the nexus of health and disease and accordingly are prominent targets for therapeutic development.^{16, 17}

Based on the significance of NOD1 and NOD2 in health and disease, their peptidoglycan ligands have been extensively derivatized to generate more effective synthetic compounds for therapeutic applications. Early studies on iE-DAP and MDP suggests these NOD1 and NOD2 agonists could stimulate neutrophils and monocytes, respectively, and facilitate the clearance of pathogens as well as tumor cells ex vivo and in vivo, but their modest potency and short half-lives limited their efficacy in animal models.¹⁷ Several studies were then initiated to improve the activity of these peptidoglycan fragments. In particular, the addition of lipophilic groups onto iE-DAP and MDP have improved their potency in cells and half-lives in vivo. For example, the addition of N-lauroyl fatty acid onto iE-DAP resulted in C12-iE-DAP, which exhibits ˜100-fold greater activation of NOD1-dependent NF-κB reporter activation.¹⁷ Similar approaches towards MDP have also yielded more potent NOD2 agonists. Notably, the addition of fatty acyl chains onto the 6-OH of N-acetylmuramic acid (MurNAc) and/or esterification of amino acids afforded more potent MDP-based NOD2 agonists.¹⁷ While these and other improved NOD1/2 agonists exhibited greater immunostimulatory activity, their efficacy as anti-infectives or adjuvants in vivo were limited. However, the development of muramyl tripeptide-phosphatidylethanolamine (MTP-PE) or mifamurtide into a liposomal formulation resulted in the drug Mepact,¹⁸ which has been approved to treat pediatric osteosarcoma in Europe. While Mepact was not approved by the FDA in USA, the collective clinical studies indicate NOD2 activation was beneficial for the preventing tumor metastases.¹⁸ Of note, microbiome profiling of cancer patients suggested immune checkpoint inhibitor therapy-responsive individuals have unique microbiota composition, which included several species of Enterococcus.¹⁹ Subsequent studies demonstrated that the Enterococcus species that expresses the unique secreted peptidoglycan hydrolase (SagA) can remodel peptidoglycan and activate NOD2 to promote immune checkpoint inhibitor cancer therapy in mice.²⁰ Moreover, the co-administration of MDP as the free molecule²⁰ or encapsulated in nanoparticles²¹ with immune checkpoint inhibitors resulted in enhanced tumor clearance in mice, suggesting improved NOD2 agonist formulation and/or composition may result in more effective immune checkpoint inhibitor therapy for the treatment of cancer and other disease indications.

Thus, there exists a need in the field for improved NOD2 agonist formulations and/or compositions for more effective immune checkpoint inhibitor therapy for the treatment of cancer and other disease indications.

### BRIEF DESCRIPTION OF THE FIGURES

FIG. 1(A-F). Virtual screening for small molecule NOD2 agonists.

FIG. 2(A-G). Structure-activity relationship studies of 3,5-dimethyl-N-arylpyrazole NOD2 agonists.

FIG. 3(A-H). NOD2 agonist TT030 activates cytokine production in vitro and improves immune checkpoint inhibitor treatment of tumor growth in vivo.

FIG. 4(A-E). NOD2 agonist TT030 remodels intratumoral immune cell composition and elicits broad inflammatory signaling.

FIG. 5(A-B). Lead 3,5-dimethyl-N-arylpyrazole TT007 specifically activates NOD2.

FIG. 6(A-E). Approach and synthesis of 3,5-dimethyl-N-arylpyrazole compound library.

FIG. 7(A-B). NOD2 agonist TT030 activates cytokine production in vitro.

FIG. 8(A-B). Single cell RNA sequencing clustering and classification.

FIG. 9(A-B). TT030 upregulates cytotoxic T cell gene expression.

### SUMMARY OF THE INVENTION

Efforts to generate more effective NOD2 agonists were undertaken through the development of desmuramyl dipeptide (dMDP) analogs, which replaced the potentially labile and dynamic structure of MurNAc with more stable and functional chemotypes.²² For example, lipophilic cinnamic acid-based dipeptide analogs have been developed as dMDP NOD2 agonists ex vivo^{23, 24} and are effective as adjuvants for promoting antigen-specific immune responses in mice²⁵. Inspired by SagA-expressing Enterococcus peptidoglycan remodeling and MDP co-administration studies with immune checkpoint inhibitor therapy,²⁰ new desmuramyl dipeptide NOD2 agonists by structure-based in silico screening and developed N-arylpyrazole dipeptides as novel NOD2 agonists, which are specific and effective at promoting immune checkpoint inhibitor therapy in vivo, were explored. These next-generation agonists as disclosed herein function as new therapeutic leads and adjuvants for a variety of NOD2-responsive diseases.

The application provides a compound of Formula I

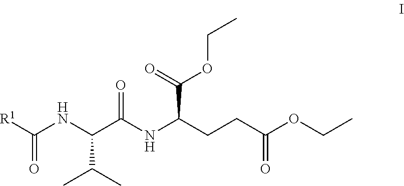

wherein:
R¹ is —(C₁-C₁₀)alkyl, —(C₁-C₁₀)heteroalkyl, —(C₂-C₁₀)alkenyl, —(C₂-C₁₀)alkynyl, —(C₂-C₁₀)heteroalkenyl, —(C₃-C₇)cycloalkyl, —(C₁-C₆)alkyl(C₃-C₇)cycloalkyl, —(C₃-C₇)cycloalkenyl,
(C₁-C₆)alkyl(C₃-C₇)cycloalkenyl, —(C₃-C₇)heterocycloalkyl, —(C₃-C₇)heterocycloalkenyl, —(C₁-C₆)alkyl(C₃-C₇)heterocycloalkyl, —(C₁-C₆)alkyl(C₃-C₇)heterocycloalkenyl, —(C₆-C₁₀)aryl, —(C₁-C₆)alkyl(C₆-C₁₀)aryl, —(C₅-C₁₀)heteroaryl, and —(C₁-C₆)alkyl(C₅-C₁₀)heteroaryl, wherein each is optionally and independently substituted with one or more R^{1′};
each R^{1′} is independently R^{1a} or R^{1b},
R^{1a} is H, halo, —CN, —OH, —NH₂, or —NHC(═O)O(C₁-C₆)alkyl;
R^{1b} is —(C₁-C₁₀)alkyl, —(C₁-C₁₀)heteroalkyl, —(C₂-C₁₀)alkenyl, —(C₂-C₁₀)alkynyl, —(C₂-C₁₀)heteroalkenyl, —(C₃-C₇)cycloalkyl, —(C₁-C₆)alkyl(C₃-C₇)cycloalkyl, —(C₃-C₇)cycloalkenyl, (C₁-C₆)alkyl(C₃-C₇)cycloalkenyl, —(C₃-C₇)heterocycloalkyl, —(C₃-C₇)heterocycloalkenyl, —(C₁-C₆)alkyl(C₃-C₇)heterocycloalkyl, —(C₁-C₆)alkyl(C₃-C₇)heterocycloalkenyl, —(C₆-C₁₀)aryl, (C₁-C₆)alkyl(C₆-C₁₀)aryl, —(C₅-C₁₀)heteroaryl, or —(C₁-C₆)alkyl(C₅-C₁₀)heteroaryl, wherein each is optionally and independently substituted with one or more R^{1″}; and
each R^{1″} is independently H, halo, oxo, —CN, —OH, —(C₁-C₆)alkyl, —O(C₁-C₆)alkyl-NH₂, —NHC(═O)O(C₁-C₆)alkyl, —(C₁-C₆)heteroalkyl, —(C₁-C₆) haloalkyl, —(C₂-C₆)alkenyl, —(C₂-C₆)alkynyl, —(C₃-C₇)cycloalkyl, —(C₃-C₇)cycloalkenyl, —(C₃-C₇)heterocycloalkyl, or —(C₃-C₇)heterocycloalkenyl;
with the proviso that Formula I is not diethyl ((E)-3-(4-hydroxy-3-methoxyphenyl)acryloyl)glycyl-L-valyl-D-glutamate; diethyl (tert-butoxycarbonyl)glycyl-L-valyl-D-glutamate; diethyl ((E)-3-(4-((tert-butoxycarbonyl)amino)phenyl)acryloyl)glycyl-L-valyl-D-glutamate; diethyl ((E)-3-(4-aminophenyl)acryloyl)glycyl-L-valyl-D-glutamate; diethyl(2-(3,4-difluorophenyl)cyclopropane-1-carbonyl)glycyl-L-valyl-D-glutamate; diethyl (6-phenyl-1H-indole-2-carbonyl)glycyl-L-valyl-D-glutamate; diethyl cinnamoylglycyl-L-valyl-D-glutamate; diethyl(2-phenylcyclopropane-1-carbonyl)glycyl-L-valyl-D-glutamate; or diethyl ((E)-3-(3,4-difluorophenyl)acryloyl)glycyl-L-valyl-D-glutamate;
or a pharmaceutically acceptable salt thereof.

The application provides a compound having the structure of Formula II

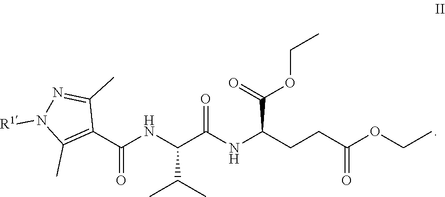

The application provides a compound having the structure of Formula III

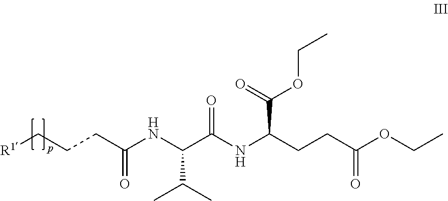

wherein:
[symbol] represents a single or double bond; and
n is 0 to 6.

The application provides a compound having the structure of Formula IV

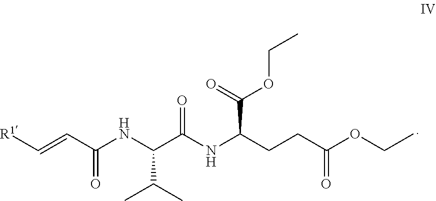

The application provides a compound having the structure of Formula V

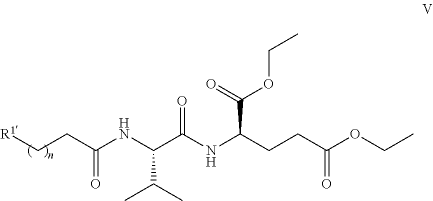

wherein:
n is 0 to 3.

The application provides a compound having the structure of Formula VI

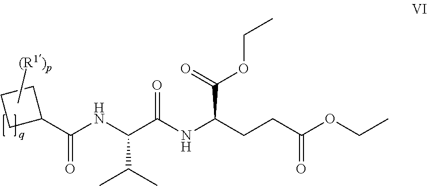

wherein:
p is 0 to 3; and
q is 0 to 3.

The application provides a pharmaceutical composition comprising the compound of any one of Formulae I-VI, admixed with a pharmaceutically acceptable carrier, diluent, or excipient.

The application provides the above pharmaceutical composition, further comprising one or more adjuvants, immunotherapeutic agents, or anti-infective compounds or compositions.

The application provides a method of activating NOD2, comprising administering to a subject infected with a NOD2-responsive infection, disease, or disorder a therapeutically effective amount of the compound of any one of Formulae I-VI or a pharmaceutical composition thereof.

The application provides a method of preventing, ameliorating, or treating a NOD2-responsive infection, disease, or disorder, comprising administering to a subject in need thereof a therapeutically effective amount of the compound of any one of Formulae I-VI or a pharmaceutical composition thereof.

### DETAILED DESCRIPTION OF THE INVENTION

PRRs represent an attractive class of drug targets due to their broad involvement in inflammation and immune regulation. Nevertheless, many synthetic approaches to design and improve PRR agonists rely on the addition of lipophilic moieties such as lipid anchors or their incorporation into novel formulations such as liposomes and other nanoparticles. Herein described is the development of a novel class of NOD2 agonists built upon the 3,5-dimethyl-N-arylpyrazole head group. This moiety has several advantages, including facile synthetic routes to access the scaffold and commercial availability of diverse precursors for structure-activity relationship studies. These factors enabled the generation and screening of multiple analogs, ultimately providing lead compounds with nanomolar EC₅₀ values in vitro. Importantly, it was found that the N-arylpyrazole group played a critical role in NOD2 binding, with multiple desmethyl analogs showing significantly decreased potency. The two methyl groups potentially play an important role in orienting the planarity of the N-arylpyrazole group with respect to the neighboring amide bond.

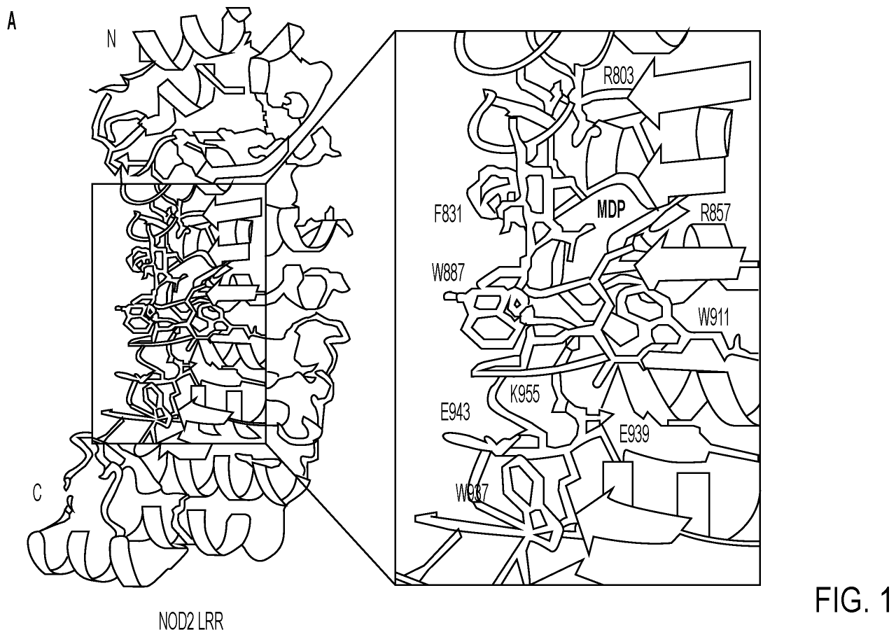

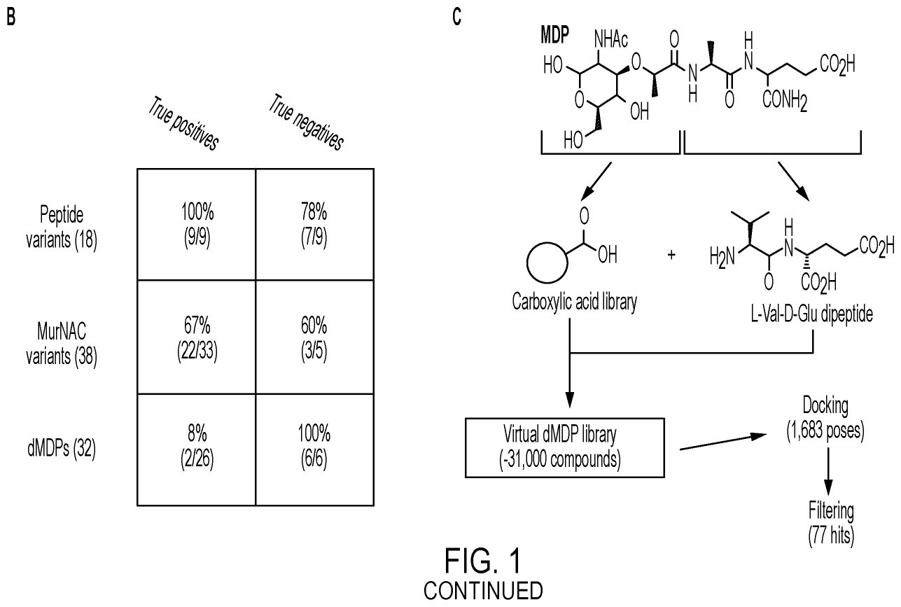

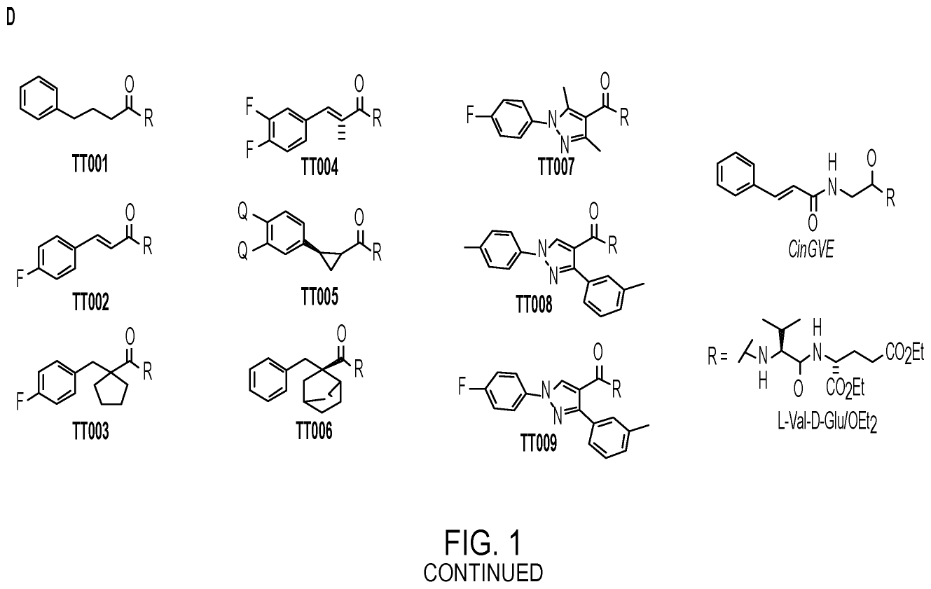

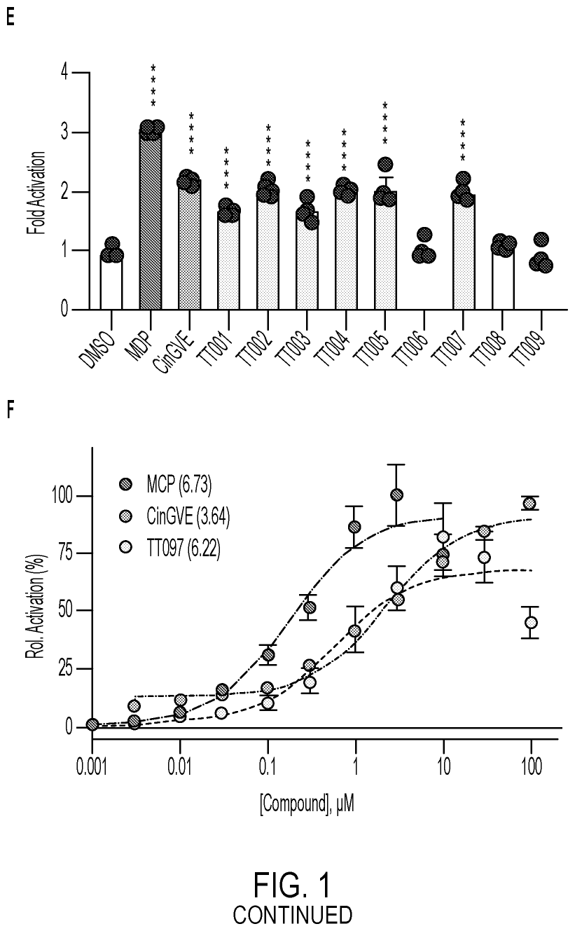

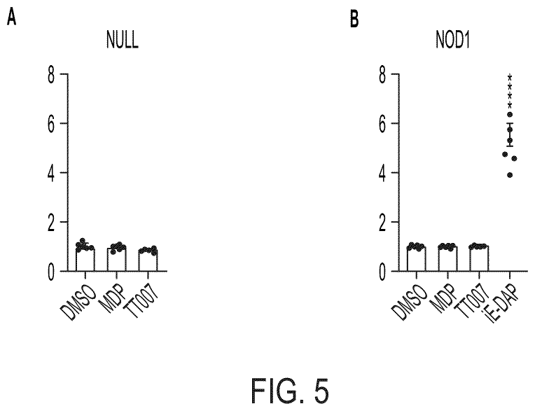

Identification of N-arylpyrazole dipeptide NOD2 agonists by structure-based in silico screening. To enable in silico screening, a powerful approach to generate new ligand scaffolds²⁶, for NOD2 agonists, whose ligand-bound structure remains unknown, MDP was docked onto the putative ligand-binding leucine-rich repeat (LRR) domain of NOD2 with the induced-fit approach²⁷ (FIG. 1A). This model shows MDP makes contacts with amino acid residues that are critical for receptor activation.²⁸⁻³⁰ To further validate the model, template-based docking of active and inactive MDP derivatives and dMDPs reported in literature was performed by aligning the maximum common substructures with the bound MDP (FIG. 1B). This resulted in the enrichment of the active derivatives in generated models. While the template-based approach was less sensitive for dMDPs, potentially indicative of the weak or moderate affinity of nearly all reported dMDPs compared to MDP, it remained highly selective for screening purposes. With the template-based docking method in hand, a library of ˜31,000 dMDPs was virtually screened (FIG. 1C). So constructed was this in silico library by conjugating enamine carboxylic acid building blocks to the dipeptide L-Val-D-Glu, which improved the activity of dMDPs relative to the native L-Ala-D-Glu and filtered the candidate ligands based on their physicochemical properties. Template docking resulted in 1,683 poses, and the top-scoring 10% were clustered and manually inspected. As further validation of this in silico method, the screening returned multiple, structurally distinct hits. In addition, some hits resembled previously reported dMDPs, including cinnamoyl-Gly-L-Val-D-Glu (OEt)₂ or CinGVE²³. A subset of the top 10% virtual screening hits encompassing the chemical diversity of the hits were synthesized and tested for activity with the NOD2-HEK-Blue NF-κB reporter cells (FIG. 1D). Six of the initial nine compounds effectively activated NOD2 with similar efficacy as CinGVE (FIG. 1E). Interestingly, one of the N-arylpyrazole compounds TT007 was active whereas the other two N-arylpyrazole compounds TT008 and TT009 were inactive, which suggested a structure-activity relationship around the pyrazole ring. Importantly, TT007 was inactive in the NULL and NOD1 HEK-Blue NF-κB reporter cells (FIG. 5), which confirmed its selectivity for NOD2, and elicited a clear dose-dependent NOD2 activation with pEC₅₀˜6.0 (FIG. 1F). Encouraged by these results, efforts were subsequently focused on the N-arylpyrazole scaffold.

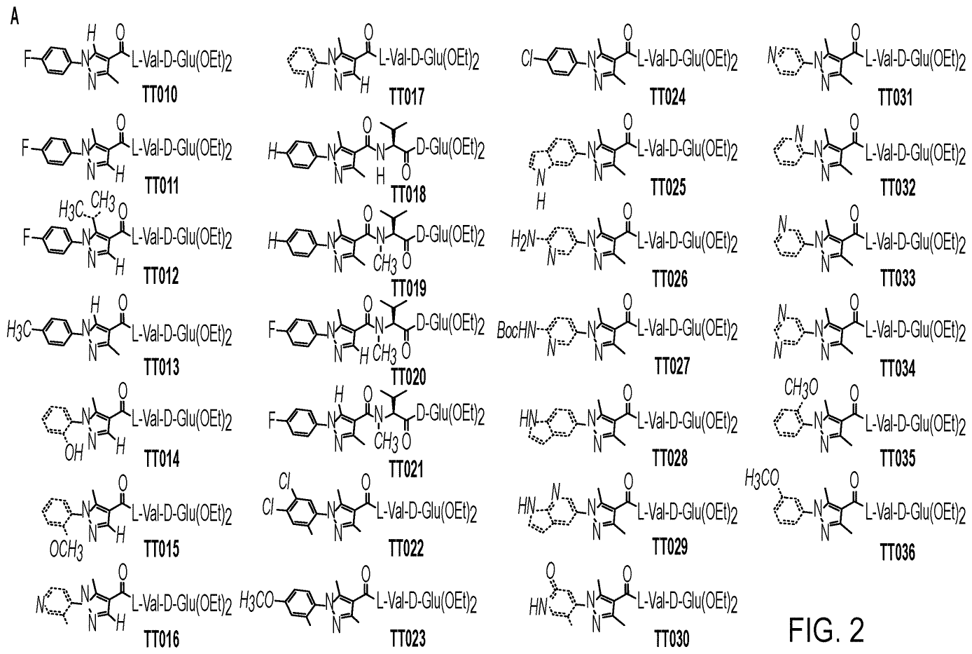

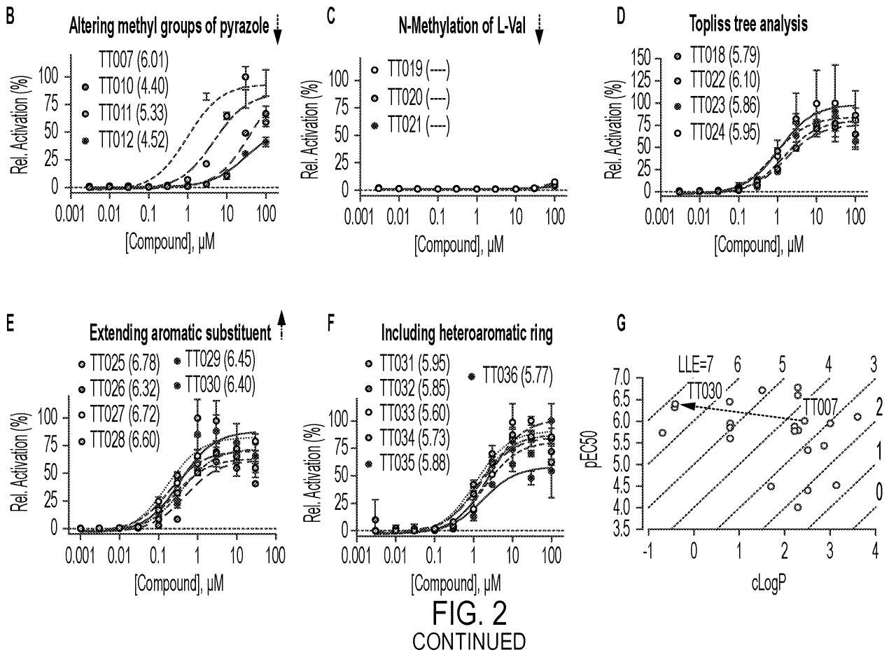

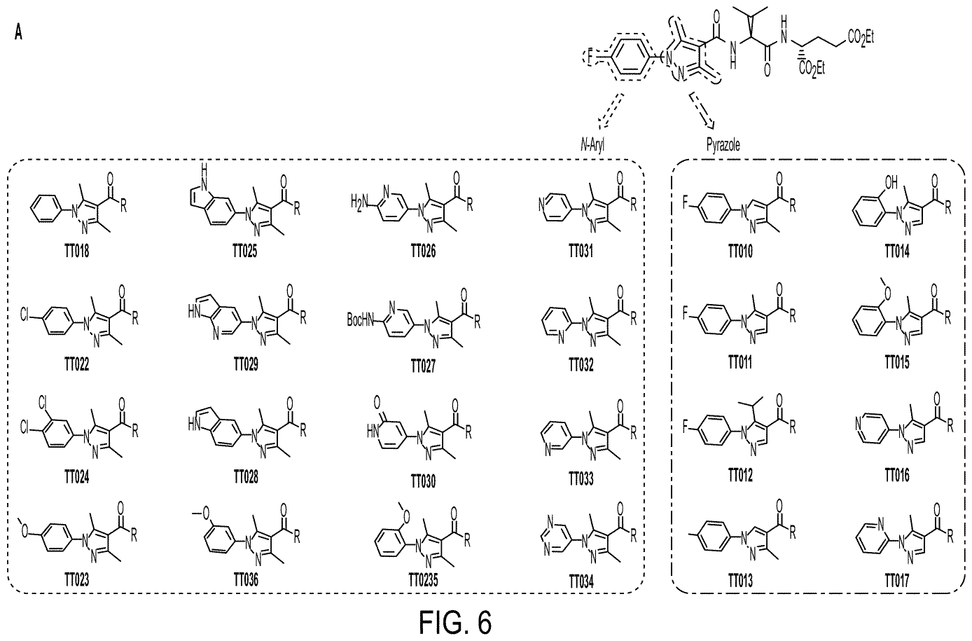

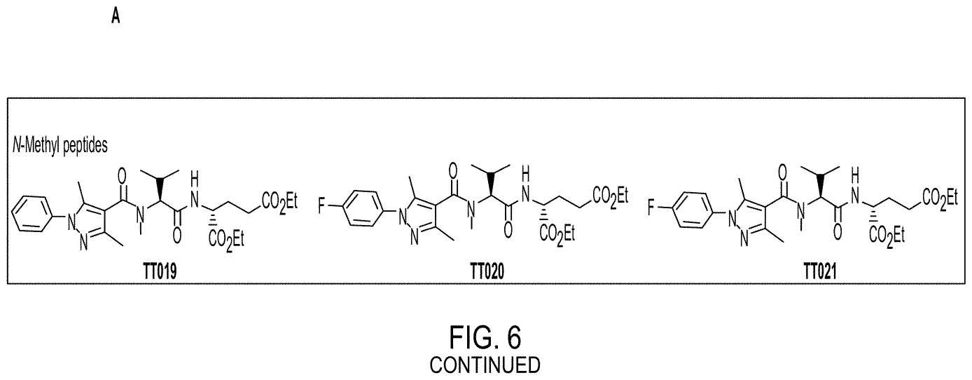

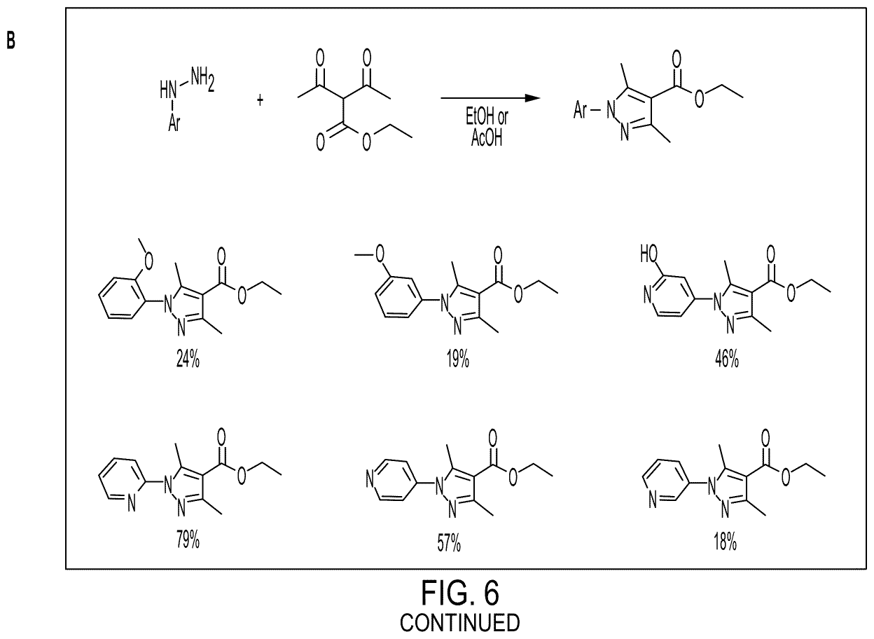

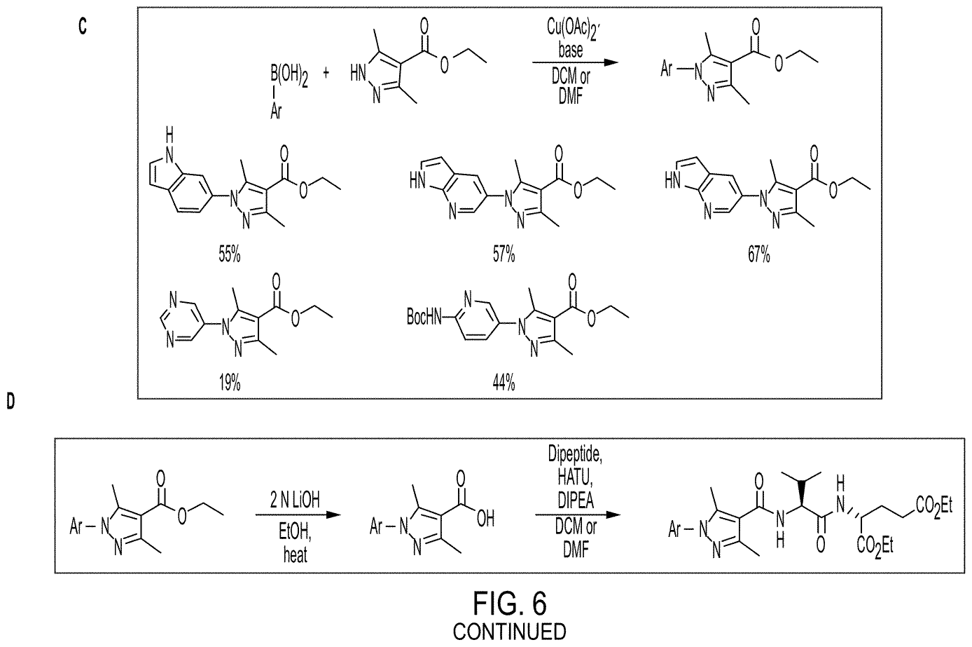

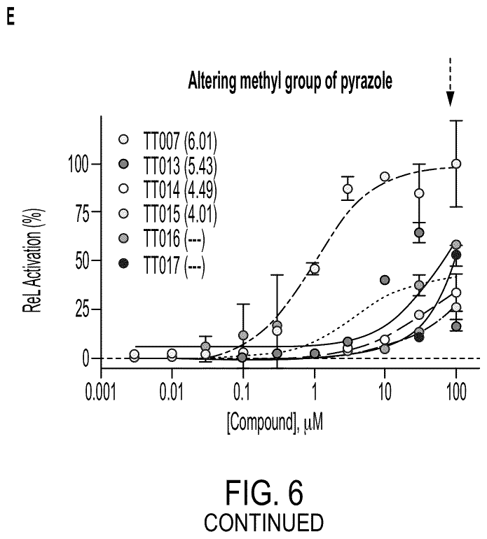

Optimization of N-arylpyrazole dipeptide NOD2 agonists. The chemical space of the N-arylpyrazole scaffold in two parts: (1) pyrazole group and (2) N-aryl group was explored (FIG. 2A and FIG. 6A). The dipeptide was synthesized as previously described and conjugated to N-arylpyrazole scaffolds that were either purchased or synthesized via Knorr pyrazole synthesis or Chan-Evans-Lam coupling routes (FIG. 6B-D). As the initial screening results suggested that substitutions on the pyrazole ring have a large effect on potency, analogs of the three N-arylpyrazole compounds with different substitutions were designed (FIG. 2B). Removing either one of the methyl groups from TT007 as in TT010 and TT011 reduced potency by up to two orders of magnitude. Increasing the size of a substituent as in TT009 and TT012 did not compensate the lack of the other substituent. This trend was also observed with compounds with various N-aryl group as in TT013-TT017 (FIG. 6E). Methyl groups can have profound effects on the binding affinity of small molecule ligands.³¹ One possible explanation for this observation may be that the two methyl groups stabilize the binding conformation by creating an energy barrier for the rotation around the bond between the aryl and pyrazole groups or the bond between the pyrazole and the amide groups. In addition, N-methylation on Val (TT018 vs. TT019, TT010 vs. TT020, TT011 vs. TT021, FIG. 2C), which was introduced to perturb the dihedral angle between the pyrazole and amide groups, abolished the activity regardless of substituents on the pyrazole ring. Analogues of TT007 with various N-aryl groups were then designed. Topliss tree approach³² as in TT018, TT022, TT023, and TT024 did not yield compounds with appreciable differences in potency except for a slight increase in TT022 possibly due to the increased steric bulk (FIG. 2D). Heteroaromatic rings that could increase steric bulk and potentially participate in polar interactions were therefore introduced. Substitutions with a hydrogen bond donor group on the para or meta position relative to the pyrazole ring as in TT025-TT030 increased potency up to ˜10-fold (FIG. 2E). On the other hand, replacing the fluorophenyl group with a hydrogen bond acceptor group such as pyridine, pyrimidine, or anisole as in TT031-TT036 did not improve potency relative to TT007 (FIG. 2F). Because MDP activity can be improved by lipid acylation¹⁷, it was important to ensure that the increased activity of these compounds was not due to a concomitant increase in lipophilicity. Comparison of the N-arylpyrazole compounds showed a moderately higher potency from the initial hit while also decreasing compound lipophilicity (FIG. 2G), with TT030 exhibiting the highest overall lipophilic ligand efficiency (LLE) of 6.81. Together, these structure-activity relationship studies led to improved potency for the N-arylpyrazole hit guided by the modification of tolerant portions of the pharmacophore while also maintaining low lipophilicity.

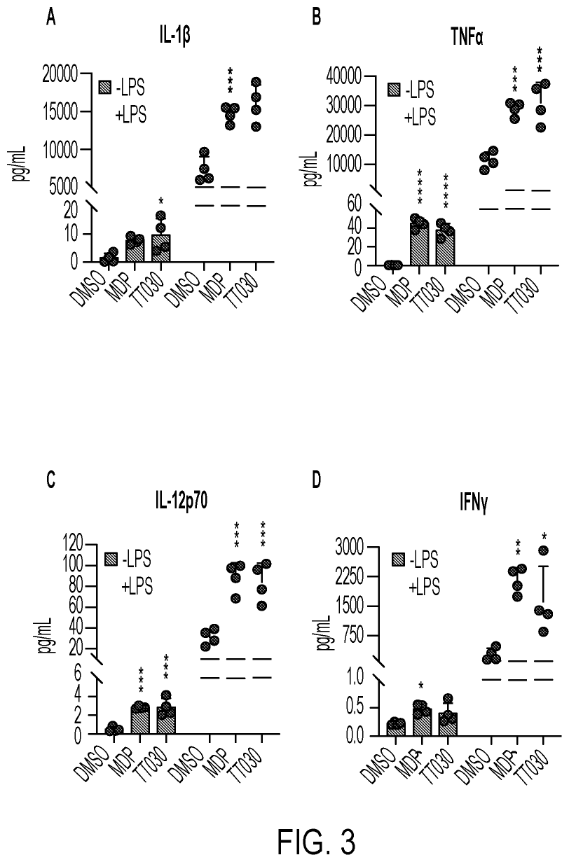

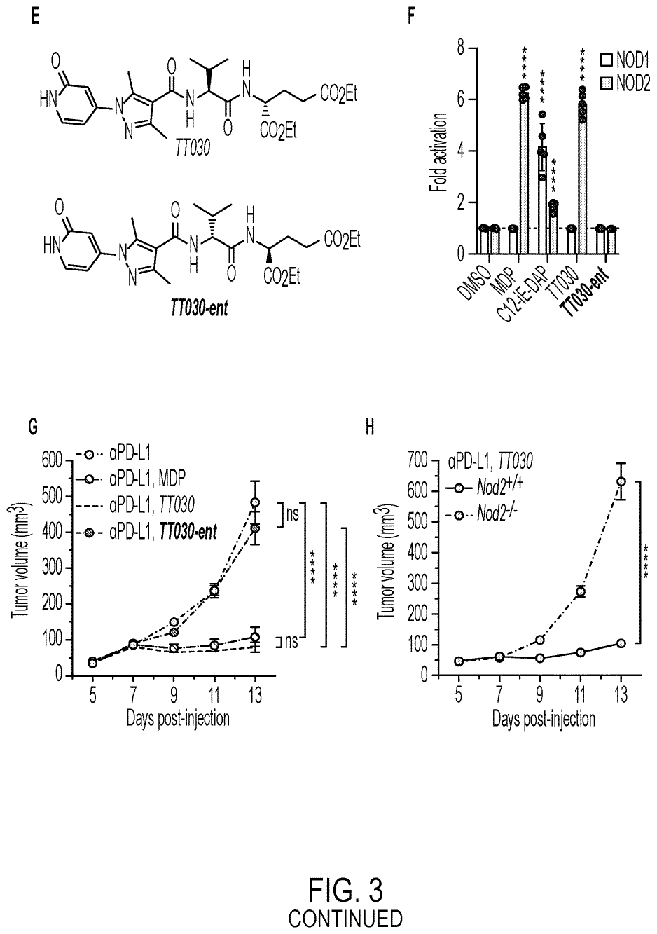

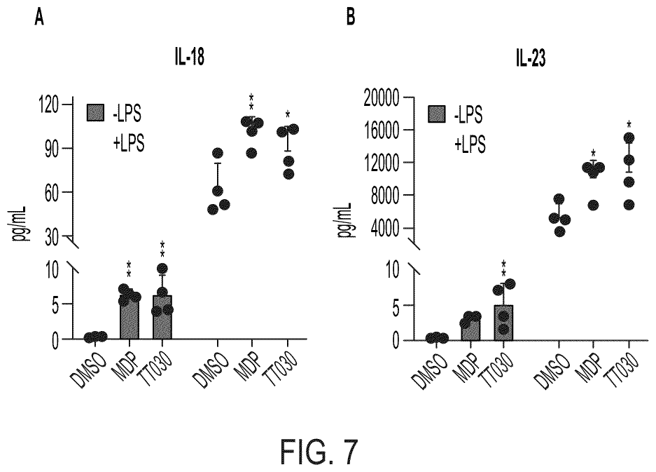

N-arylpyrazole NOD2 agonist promotes cytokine production ex vivo. To screen NOD2 activation in a more physiologically relevant system, the cytokine release profile of the optimized lead TT030 using primary human immune cells was examined. Healthy human peripheral mononuclear blood cells (PBMCs) were first depleted of red blood cells and then stimulated overnight with vehicle, MDP, or TT030. Because bacterial lipopolysaccharide has been reported to amplify cytokine secretion caused by NOD2 stimulation, the experiment in parallel with LPS co-stimulation was also conducted. As expected, MDP stimulation led to a significant increase of canonical, NOD2-dependent cytokines in the medium after 24 h, including interleukin-1 beta (IL-1B) (FIG. 3A) and tumor necrosis factor alpha (TNFα) (FIG. 3B). Similarly, TT030 was able to induce equivalent concentrations of these cytokines. In addition, TT030 caused an increase in IL-12p70 secretion with and without LPS (FIG. 3C), as well as the production of the IL-1 family member IL-18 (FIG. 7A) and the IL-12 family member IL-23 (FIG. 7B), highlighting the broad impact of NOD2 activation on the overall cytokine environment. Importantly, IL-12 family cytokines lead to the production of interferon gamma (IFNγ) by activated T and NK cells to enhance adaptive immune responses, and increased IFNγ levels were also observed for both MDP and TT030 stimulation (FIG. 3D).

N-arylpyrazole NOD2 agonist promotes immunotherapy in vivo. Next compared was TT030 activity against MDP in vivo. Previous work had demonstrated that MDP can potentiate the activity of anti-PD-L1 checkpoint inhibitor treatment of a subcutaneous B16-F10 melanoma model in a NOD2-dependent manner.²⁰ Using a similar treatment model, mice inoculated with B16 tumors were co-administered with anti-PD-L1 and either MDP or TT030. In addition, the enantiomer of TT030 (TT030-ent) was chemically synthesized (FIG. 3E) and shown to be NOD2-inactive using the HEK-Blue in vitro assay (FIG. 3F). As a control compound, TT030-ent was administered to a separate cohort of tumor-bearing animals along with the checkpoint inhibitor. As anticipated, administration of anti-PD-L1 alone was not effective at controlling B16 tumor growth over time, whereas co-treatment with MDP significantly inhibited tumor growth (FIG. 3G). This anti-tumor activity was also observed for the TT030 but not for TT030-ent, indicating the N-arylpyrazole NOD2 agonist was active in vivo and enantiomer-specific. This experiment in wild-type and Nod2^{-/-} mice was then repeated (FIG. 3H). NOD2 knockout caused a loss in the anti-tumor activity observed in the wild-type cohort, demonstrating that NOD2 was required for the ability of TT030 to augment anti-PD-L1 immunotherapy in vivo.

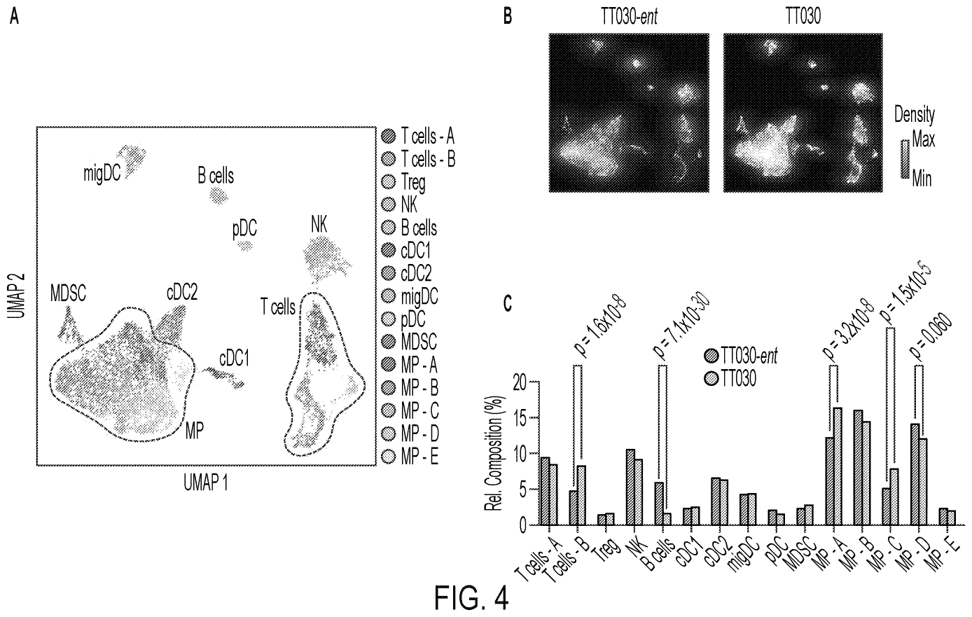

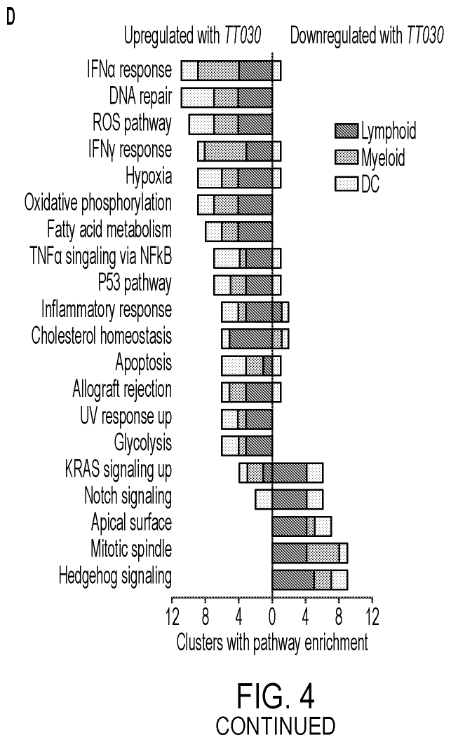

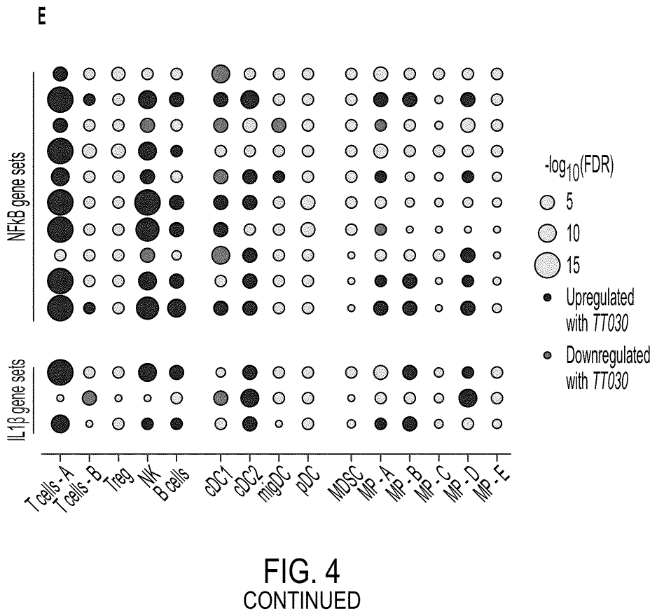

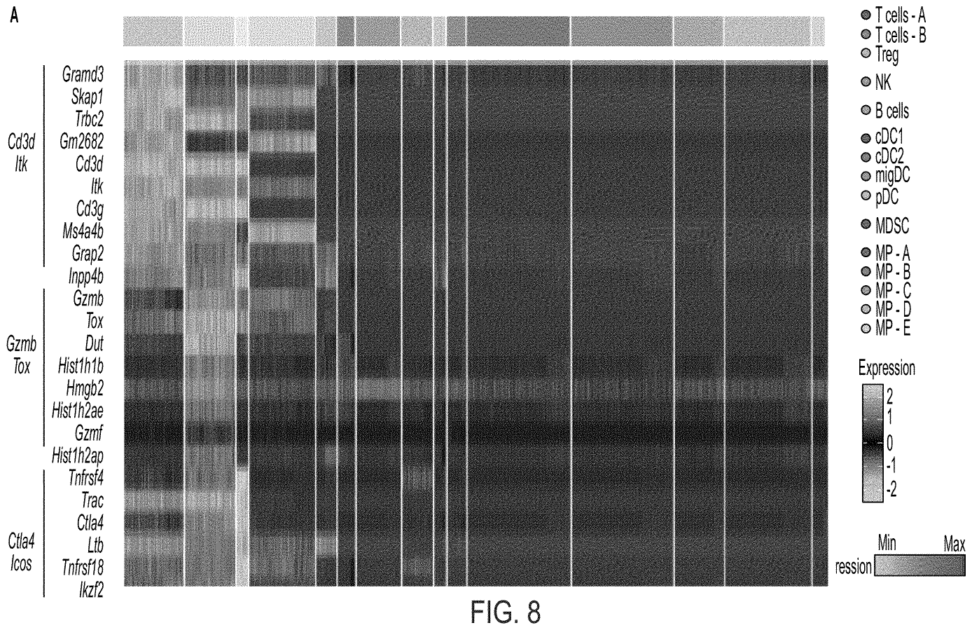

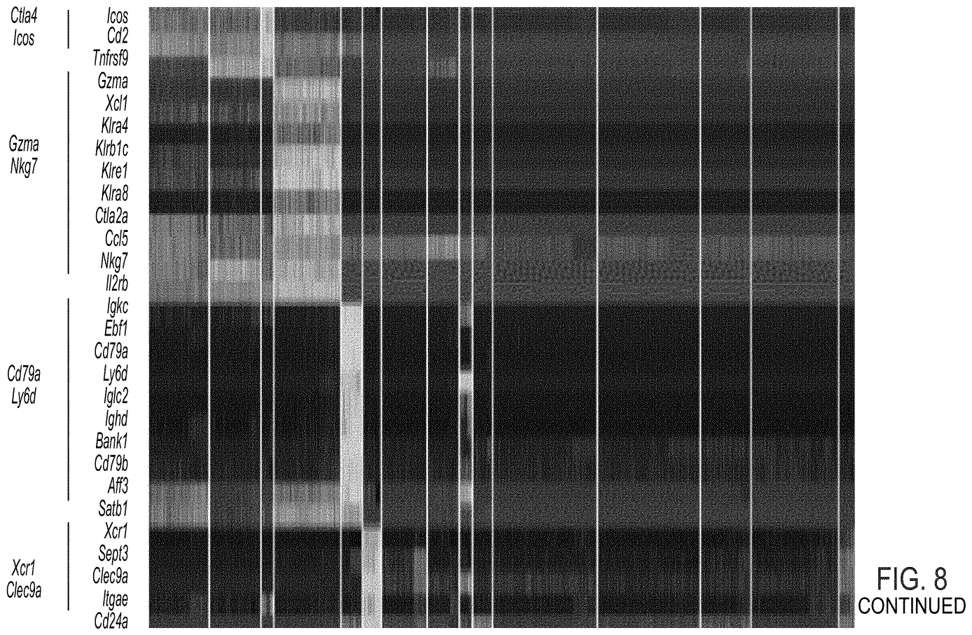

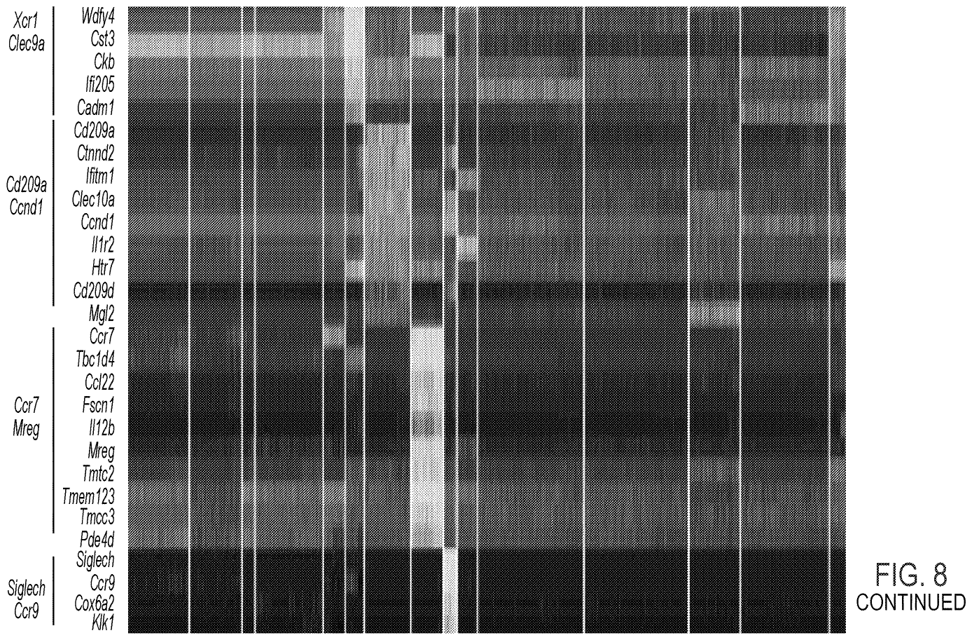

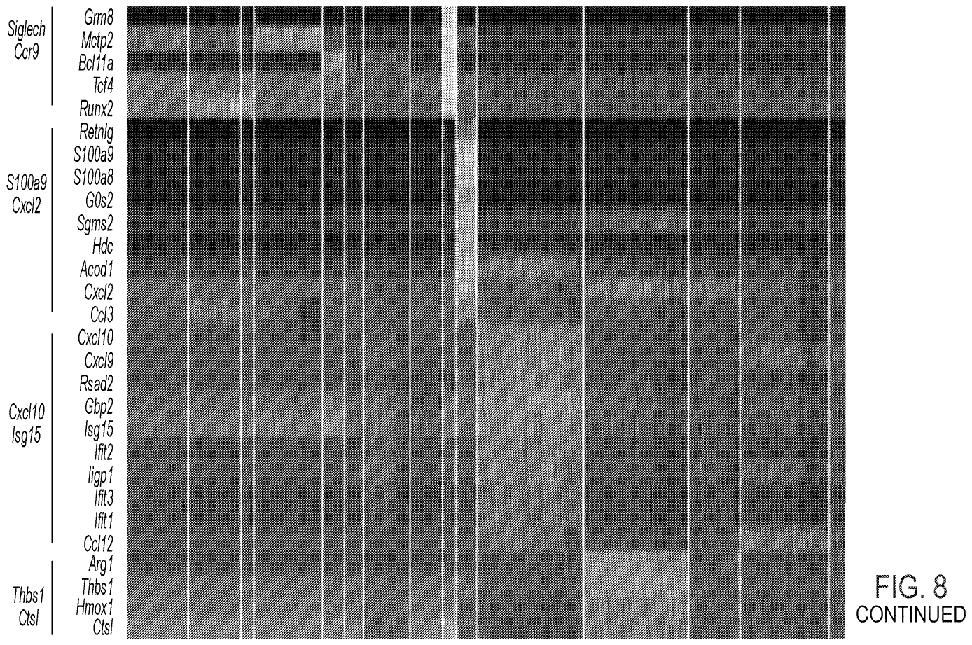

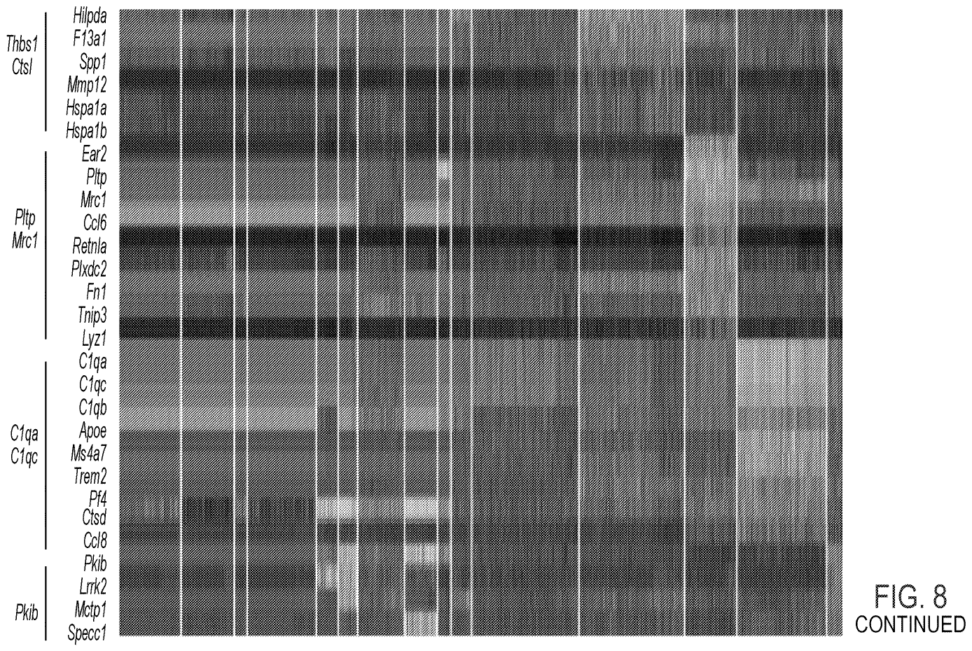

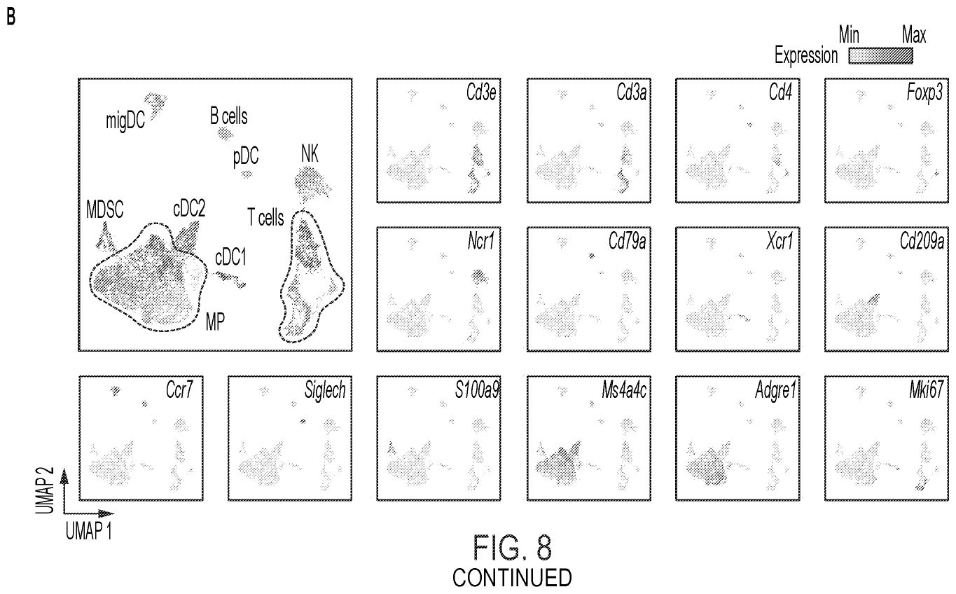

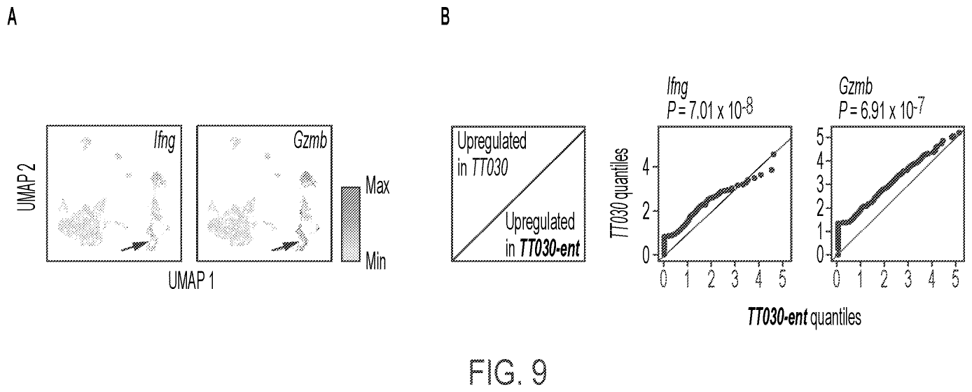

N-arylpyrazole NOD2 agonist enhances intratumoral immune responses in vivo. The anti-tumor mechanism of action of the N-arylpyrazole NOD2 agonist in vivo was then further characterized. Infiltrating leukocytes from tumors treated with anti-PD-L1 and either TT030 or TT030-ent were harvested via cell sorting and subjected to single cell transcriptomic profiling (FIG. 4A and FIG. 8). After assignment of cell clusters using well-defined markers, the overall composition of intratumoral immune cells were examined by comparing the relative proportions of each cell cluster. Here, it was found that tumors treated with TT030 showed a significant increase in the amount of a T cell cluster characterized by the expression of Cd8a, Ifng, Gzmb, and Mki67 (FIGS. 4B, 4C and FIGS. 8 and 9A), suggesting that the TT030 sample contained a higher amount of dividing cytotoxic T cells. In fact, overall expression of both Ifng and Gzmb were elevated in the TT030 sample compared to the enantiomer control (FIG. 9B), indicating heighted CD8⁺ T cell activation and effector function. Shifts in multiple clusters of mononuclear phagocytes (monocytes and macrophages) (FIGS. 4B, 4C) were also observed, which indicated that TT030 stimulation could also remodel the intratumoral myeloid compartment. To better characterize changes to downstream signaling caused by TT030, differential gene expression and gene set enrichment analyses were performed. Numerous inflammatory, immune activation, and metabolic pathways were upregulated in multiple clusters from tumors treated with TT030, including IFNα and IFNγ response as well as NF-κB signaling (FIG. 4D). As mentioned above, NOD2 initiates transcriptional changes through the transcription factor NF-κB, which were observed with broad enrichment in REACTOME pathways annotated for NF-κB activation (FIG. 4E). Enrichment in REACTOME pathways annotated for signaling via IL-1β (FIG. 4E) was also found, indicating that TT030 activated the canonical NOD2-NF-κB-IL-1β signaling axis within the tumor microenvironment akin to these results ex vivo and previous studies with MDP in vivo²⁰.

The results disclosed herein indicate that the N-arylpyrazole NOD2 agonist TT030 functions similarly to MDP, the naturally occurring molecular pattern for NOD2. Both MDP and TT030 elicit a variety of cytokines that mediate innate and adaptive immune responses, including IL-1β, TNFα, and IL-12p70. TT030 also exhibited the equivalent anti-tumor activity as MDP in combination with anti-PD-L1. That MDP can synergize with antibodies against all clinically approved checkpoint targets has previously been demonstrated; therefore, the N-arylpyrazole NOD2 agonist may also function as an adjuvant for multiple checkpoint inhibitors. A recent report has indicated that phosphorylation of MDP at the 6-O-hydroxyl position by N-acetylglucosamine kinase (NAGK) is required for NOD2 activation using Nagk^{-/-} mammalian cells ex vivo.³³ These results, along with other reports of desmuramyl NOD2 agonists that lack a potential phosphorylation site²²⁻²⁵, suggest that synthetic agonists can engage NOD2 without further modification, and the possibility of multiple binding poses of NOD2 agonists. Importantly, the activity observed in the system disclosed herein required both the proper stereochemistry of the N-arylpyrazole dipeptide as well as the expression of host NOD2, providing further evidence for its predicted, on-target activity. Finally, alterations in intratumoral immune cell composition and activation mirror those observed with MDP stimulation, including increased levels of CD8⁺ effector T cells and activation of NF-κB and IL-1β signaling. Altogether, these results support TT030 and the 3,5-dimethyl-N-arylpyrazole scaffold as a potent lead compound and chemotype to stimulate NOD2 signaling for eliciting both innate and adaptive immune responses in vivo for immunotherapy.

### REFERENCES

1 Takeuchi, O. & Akira, S. Pattern recognition receptors and inflammation. Cell 140, 805-820 (2010).
2. Anwar, M. A., Shah, M., Kim, J. & Choi, S. Recent clinical trends in Toll-like receptor targeting therapeutics. Med Res Rev 39, 1053-1090 (2019).
3 Jones, J. D., Vance, R. E. & Dangl, J. L. Intracellular innate immune surveillance devices in plants and animals. Science 354 (2016).
4. Philpott, D. J., Sorbara, M. T., Robertson, S. J., Croitoru, K. & Girardin, S. E. NOD proteins: regulators of inflammation in health and disease. Nat Rev Immunol 14, 9-23 (2014).
5. Caruso, R., Warner, N., Inohara, N. & Nunez, G. NOD1 and NOD2: signaling, host defense, and inflammatory disease. Immunity 41, 898-908 (2014).
6. Ellouz, F., Adam, A., Ciorbaru, R. & Lederer, E. Minimal structural requirements for adjuvant activity of bacterial peptidoglycan derivatives. Biochem Biophys Res Commun 59, 1317-1325 (1974).
7. Girardin, S. E. et al. Peptidoglycan molecular requirements allowing detection by Nod1 and Nod2. J Biol Chem 278, 41702-41708 (2003).
8. Inohara, N. et al. Host recognition of bacterial muramyl dipeptide mediated through NOD2. Implications for Crohn's disease. J Biol Chem 278, 5509-5512 (2003).
9. Kobayashi, K. S. et al. Nod2-dependent regulation of innate and adaptive immunity in the intestinal tract. Science 307, 731-734 (2005).
10. Girardin, S. E. et al. Nod1 detects a unique muropeptide from gram-negative bacterial peptidoglycan. Science 300, 1584-1587 (2003).
11. Chamaillard, M. et al. An essential role for NOD1 in host recognition of bacterial peptidoglycan containing diaminopimelic acid. Nat Immunol 4, 702-707 (2003).
12. Trindade, B. C. & Chen, G. Y. NOD1 and NOD2 in inflammatory and infectious diseases. Immunol Rev 297, 139-161 (2020).
13. Keestra-Gounder, A. M. & Tsolis, R. M. NOD1 and NOD2: Beyond Peptidoglycan Sensing. Trends Immunol 38, 758-767 (2017).
14. Pei, G. et al. Cellular stress promotes NOD1/2-dependent inflammation via the endogenous metabolite sphingosine-1-phosphate. EMBO J 40, e106272 (2021).
15. Gabanyi, I. et al. Bacterial sensing via neuronal Nod2 regulates appetite and body temperature. Science 376, eabj3986 (2022).
16. Geddes, K., Magalhaes, J. G. & Girardin, S. E. Unleashing the therapeutic potential of NOD-like receptors. Nat Rev Drug Discov 8, 465-479 (2009).
17. Griffin, M. E., Hespen, C. W., Wang, Y. C. & Hang, H. C. Translation of peptidoglycan metabolites into immunotherapeutics. Clin Transl Immunology 8, e1095 (2019).
18. Meyers, P. A. & Chou, A. J. Muramyl tripeptide-phosphatidyl ethanolamine encapsulated in liposomes (L-MTP-PE) in the treatment of osteosarcoma. Adv Exp Med Biol 804, 307-321 (2014).
19. Helmink, B. A., Khan, M. A. W., Hermann, A., Gopalakrishnan, V. & Wargo, J. A. The microbiome, cancer, and cancer therapy. Nat Med 25, 377-388 (2019).
20. Griffin, M. E. et al. Enterococcus peptidoglycan remodeling promotes checkpoint inhibitor cancer immunotherapy. Science 373, 1040-1046 (2021).
21. Priem, B. et al. Trained Immunity-Promoting Nanobiologic Therapy Suppresses Tumor Growth and Potentiates Checkpoint Inhibition. Cell 183, 786-801 e719 (2020).
22. Nabergoj, S., Mlinaric-Rascan, I. & Jakopin, Z. Harnessing the untapped potential of nucleotide-binding oligomerization domain ligands for cancer immunotherapy. Med Res Rev 39, 1447-1484 (2019).
23. Gobec, M. et al. Discovery of Nanomolar Desmuramylpeptide Agonists of the Innate Immune Receptor Nucleotide-Binding Oligomerization Domain-Containing Protein 2 (NOD2) Possessing Immunostimulatory Properties. J Med Chem 61, 2707-2724 (2018).
24. Guzelj, S., Bizjak, S. & Jakopin, Z. Discovery of Desmuramylpeptide NOD2 Agonists with Single-Digit Nanomolar Potency. ACS Med Chem Lett 13, 1270-1277 (2022).
25. Guzelj, S. et al. Structural Fine-Tuning of Desmuramylpeptide NOD2 Agonists Defines Their In Vivo Adjuvant Activity. J Med Chem 64, 7809-7838 (2021).
26. Shoichet, B. K. Virtual screening of chemical libraries. Nature 432, 862-865 (2004).
27. Sherman, W., Day, T., Jacobson, M. P., Friesner, R. A. & Farid, R. Novel procedure for modeling ligand/receptor induced fit effects. J Med Chem 49, 534-553 (2006).
28. D′Ambrosio, E. A., Bersch, K. L., Lauro, M. L. & Grimes, C. L. Differential Peptidoglycan Recognition Assay Using Varied Surface Presentations. J Am Chem Soc 142, 10926-10930 (2020).
29. Maekawa, S., Ohto, U., Shibata, T., Miyake, K. & Shimizu, T. Crystal structure of NOD2 and its implications in human disease. Nat Commun 7, 11813 (2016).
30. Tanabe, T. et al. Regulatory regions and critical residues of NOD2 involved in muramyl dipeptide recognition. EMBO J 23, 1587-1597 (2004).
31. Schonherr, H. & Cernak, T. Profound methyl effects in drug discovery and a call for new C—H methylation reactions. Angew Chem Int Ed Engl 52, 12256-12267 (2013).
32. Richter, L. Topliss Batchwise Schemes Reviewed in the Era of Open Data Reveal Significant Differences between Enzymes and Membrane Receptors. J Chem Inf Model 57, 2575-2583 (2017).
33. Stafford, C. A. et al. Phosphorylation of muramyl peptides by NAGK is required for NOD2 activation. Nature (2022).

### Embodiments

Embodiment 1. A compound of Formula I

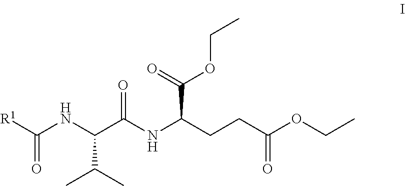

wherein:
R¹ is —(C₁-C₁₀)alkyl, —(C₁-C₁₀)heteroalkyl, —(C₂-C₁₀)alkenyl, —(C₂-C₁₀)alkynyl, —(C₂-C₁₀)heteroalkenyl, —(C₃-C₇)cycloalkyl, —(C₁-C₆)alkyl(C₃-C₇)cycloalkyl, —(C₃-C₇)cycloalkenyl, —(C₁-C₆)alkyl(C₃-C₇)cycloalkenyl, —(C₃-C₇)heterocycloalkyl, —(C₃-C₇)heterocycloalkenyl, —(C₁-C₆)alkyl(C₃-C₇)heterocycloalkyl, —(C₁-C₆)alkyl(C₃-C₇)heterocycloalkenyl, —(C₆-C₁₀)aryl, —(C₁-C₆)alkyl(C₆-C₁₀)aryl, —(C₅-C₁₀)heteroaryl, and —(C₁-C₆)alkyl(C₅-C₁₀)heteroaryl, wherein each is optionally and independently substituted with one or more R^{1′};
each R^{1′} is independently R^{1a} or R^{1b},
R^{1a} is H, halo, —CN, —OH, —NH₂, or —NHC(═O)O(C₁-C₆)alkyl;
R^{1b} is —(C₁-C₁₀)alkyl, —(C₁-C₁₀)heteroalkyl, —(C₂-C₁₀)alkenyl, —(C₂-C₁₀)alkynyl, —(C₂-C₁₀)heteroalkenyl, —(C₃-C₇)cycloalkyl, —(C₁-C₆)alkyl(C₃-C₇)cycloalkyl, —(C₃-C₇)cycloalkenyl, —(C₁-C₆)alkyl(C₃-C₇)cycloalkenyl, —(C₃-C₇)heterocycloalkyl, —(C₃-C₇)heterocycloalkenyl, —(C₁-C₆)alkyl(C₃-C₇)heterocycloalkyl, —(C₁-C₆)alkyl(C₃-C₇)heterocycloalkenyl, —(C₆-C₁₀)aryl, —(C₁-C₆)alkyl(C₆-C₁₀)aryl, —(C₅-C₁₀)heteroaryl, or —(C₁-C₆)alkyl(C₅-C₁₀)heteroaryl, wherein each is optionally and independently substituted with one or more R^{1″}; and
each R^{1″} is independently H, halo, oxo, —CN, —OH, —(C₁-C₆)alkyl, —O(C₁-C₆)alkyl-NH₂, —NHC(═O)O(C₁-C₆)alkyl, —(C₁-C₆)heteroalkyl, —(C₁-C₆) haloalkyl, —(C₂-C₆)alkenyl, —(C₂-C₆)alkynyl, —(C₃-C₇)cycloalkyl, —(C₃-C₇)cycloalkenyl, —(C₃-C₇)heterocycloalkyl, or —(C₃-C₇)heterocycloalkenyl;
with the proviso that Formula I is not diethyl ((E)-3-(4-hydroxy-3-methoxyphenyl)acryloyl)glycyl-L-valyl-D-glutamate; diethyl (tert-butoxycarbonyl)glycyl-L-valyl-D-glutamate; diethyl ((E)-3-(4-((tert-butoxycarbonyl)amino)phenyl)acryloyl)glycyl-L-valyl-D-glutamate; diethyl ((E)-3-(4-aminophenyl)acryloyl)glycyl-L-valyl-D-glutamate; diethyl(2-(3,4-difluorophenyl)cyclopropane-1-carbonyl)glycyl-L-valyl-D-glutamate; diethyl (6-phenyl-1H-indole-2-carbonyl)glycyl-L-valyl-D-glutamate; diethyl cinnamoylglycyl-L-valyl-D-glutamate; diethyl(2-phenylcyclopropane-1-carbonyl)glycyl-L-valyl-D-glutamate; or diethyl ((E)-3-(3,4-difluorophenyl)acryloyl)glycyl-L-valyl-D-glutamate;
or a pharmaceutically acceptable salt thereof.

Embodiment 2. The compound of Embodiment 1, wherein R¹ is —(C₁-C₁₀)alkyl optionally substituted with one or more R^{1′}.

Embodiment 3. The compound of Embodiment 1, wherein R¹ is —(C₂-C₁₀)alkenyl optionally substituted with one or more R^{1′}.

Embodiment 4. The compound of Embodiment 1, wherein R¹ is —(C₃-C₇)cycloalkyl optionally substituted with one or more R^{1′}.

Embodiment 5. The compound of Embodiment 4, wherein-(C₃-C₇)cycloalkyl is cyclopropyl.

Embodiment 6. The compound of Embodiment 4, wherein-(C₃-C₇)cycloalkyl is cyclopentyl.

Embodiment 7. The compound of Embodiment 1, wherein R¹ is —(C₁-C₆)alkyl(C₃-C₇)heterocycloalkenyl optionally substituted with one or more R^{1′}.

Embodiment 8. The compound of Embodiment 7, wherein-(C₁-C₆)alkyl(C₃-C₇)heterocycloalkenyl is methylene diazirinyl.

Embodiment 9. The compound of Embodiment 1, wherein R¹ is —(C₅-C₁₀)heteroaryl optionally substituted with one or more R^{1′}.

Embodiment 10. The compound of Embodiment 9, wherein-(C₅-C₁₀)heteroaryl is pyrazolyl.

Embodiment 11. The compound of Embodiment 10, wherein R¹ is pyrazolyl optionally substituted with one or more —(C₁-C₆)alkyl and further substituted with one or more R^{1′}.

Embodiment 12. The compound of Embodiment 11, wherein R¹ is pyrazolyl substituted with one or more —CH₃ and further substituted with one or more R^{1′}.

Embodiment 13. The compound of Embodiment 12, wherein R¹ is pyrazolyl substituted with one or more —CH₃ and further substituted with one or more R^{1′}.

Embodiment 14. The compound of Embodiment 13, wherein R¹ is methyl pyrazolyl further substituted with one or more R^{1′}.

Embodiment 15. The compound of Embodiment 13, wherein R¹ is dimethyl pyrazolyl further substituted with one or more R^{1′}.

Embodiment 16. The compound of Embodiment 1, having the structure of Formula II

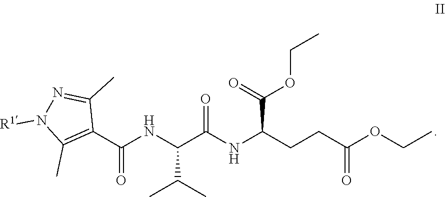

Embodiment 17. The compound of Embodiment 1, having the structure of Formula III

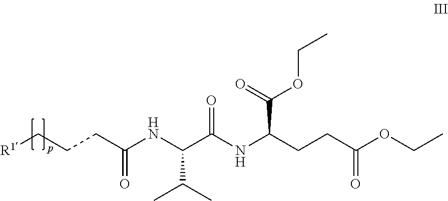

wherein:
[symbol] represents a single or double bond; and
n is 0 to 6.

Embodiment 18. The compound of Embodiment 17, wherein n is 0.

Embodiment 19. The compound of Embodiment 17, wherein n is 1.

Embodiment 20. The compound of Embodiment 17, wherein n is 2.

Embodiment 21. The compound of any one of Embodiments 17-20, wherein [symbol] is a single bond.

Embodiment 22. The compound of any one of Embodiments 17-20, wherein [symbol] is a double bond.

Embodiment 23. The compound of Embodiment 1, having the structure of Formula IV

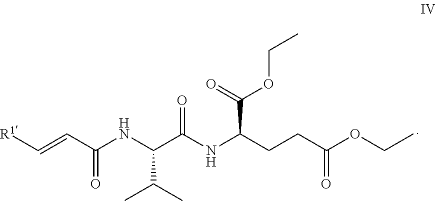

Embodiment 24. The compound of Embodiment 1, having the structure of Formula V

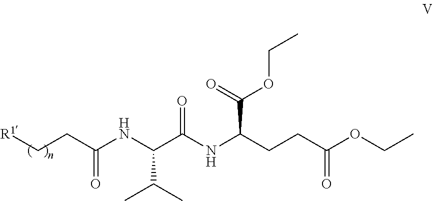

wherein:
n is 0 to 3.

Embodiment 25. The compound of Embodiment 24, wherein n is 0.

Embodiment 26. The compound of Embodiment 24, wherein n is 1.

Embodiment 27. The compound of Embodiment 24, wherein n is 2.

Embodiment 28. The compound of Embodiment 1, having the structure of Formula VI

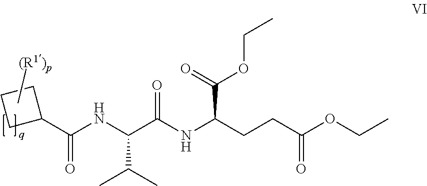

wherein:
p is 0 to 3; and
q is 0 to 3.

Embodiment 29. The compound of Embodiment 28, wherein q is 0 and p is 1.

Embodiment 30. The compound of Embodiment 28, wherein q is 1 and p is 1.

Embodiment 31. The compound of Embodiment 28, wherein q is 2 and p is 1.

Embodiment 32. The compound of any one of Embodiments 1-31, wherein R^{1′} is Ph optionally substituted with one or more R^{1″}.

Embodiment 33. The compound of any one of Embodiments 1-31, wherein R^{1′} is —(C₅-C₁₀)heteroaryl optionally substituted with one or more R^{1″}.

Embodiment 34. The compound of Embodiment 33, wherein R^{1′} is pyridinyl optionally substituted with one or more R^{1″}.

Embodiment 35. The compound of Embodiment 33, wherein R^{1′} is 1,2-dihydropyridinyl optionally substituted with one or more R^{1″}.

Embodiment 36. The compound of Embodiment 33, wherein R^{1′} is pyrimidinyl optionally substituted with one or more R^{1″}.

Embodiment 37. The compound of Embodiment 33, wherein R^{1′} is 1-H-indolyl optionally substituted with one or more R^{1″}.

Embodiment 38. The compound of Embodiment 33, wherein R^{1′} is 1-H-pyrrolo[2,3-b]pyridinyl optionally substituted with one or more R^{1″}.

Embodiment 39. The compound of any one of Embodiments 1-39, wherein one R^{1″} is present.

Embodiment 40. The compound of Embodiment 39, wherein R^{1″} is halo.

Embodiment 41. The compound of Embodiment 40, wherein halo is F.

Embodiment 42. The compound of Embodiment 40, wherein halo is Cl.

Embodiment 43. The compound of Embodiment 39, wherein R^{1″} is —OMe.

Embodiment 44. The compound of Embodiment 39, wherein R^{1″} is —NH₂.

Embodiment 45. The compound of Embodiment 35, wherein R^{1″} is oxo.

Embodiment 46. The compound of Embodiment 39, wherein R^{1″} is oxo.

Embodiment 47. The compound of Embodiment 39, wherein R^{1″} is Me.

Embodiment 48. The compound of Embodiment 39, wherein R^{1″} is —OH.

Embodiment 49. The compound of any one of Embodiments 1-38, wherein two R^{1″} are present.

Embodiment 50. The compound of Embodiment 49, wherein at least one R^{1″} is halo.

Embodiment 51. The compound of Embodiment 49, wherein both R^{1″} are halo.

Embodiment 52. The compound of either Embodiment 49 or Embodiment 50, wherein halo is F.

Embodiment 53. The compound of either Embodiment 49 or Embodiment 50, wherein halo is Cl.

Embodiment 54. A compound having any one of the formulae selected from the group consisting of:
diethyl (4-phenylbutanoyl)-L-valyl-D-glutamate;
diethyl ((E)-3-(4-fluorophenyl)acryloyl)-L-valyl-D-glutamate;
diethyl (1-(4-fluorobenzyl)cyclopentane-1-carbonyl)-L-valyl-D-glutamate;
rac-diethyl ((1R,2R)-2-(3,4-dichlorophenyl)cyclopropane-1-carbonyl)-L-valyl-D-glutamate;
rac-diethyl ((R)-3-(3,4-difluorophenyl)-2-methylpropanoyl)-L-valyl-D-glutamate;
diethyl (1-(4-fluorophenyl)-3,5-dimethyl-1H-pyrazole-4-carbonyl)-L-valyl-D-glutamate;
diethyl (1-(4-fluorophenyl)-5-methyl-1H-pyrazole-4-carbonyl)-L-valyl-D-glutamate;
diethyl (1-(4-fluorophenyl)-3-methyl-1H-pyrazole-4-carbonyl)-L-valyl-D-glutamate;
diethyl (1-(4-fluorophenyl)-5-isopropyl-1H-pyrazole-4-carbonyl)-L-valyl-D-glutamate;
diethyl (3-methyl-1-(p-tolyl)-1H-pyrazole-4-carbonyl)-L-valyl-D-glutamate;
diethyl (1-(2-hydroxyphenyl)-5-methyl-1H-pyrazole-4-carbonyl)-L-valyl-D-glutamate;
diethyl (5-methyl-1-(pyridin-4-yl)-1H-pyrazole-4-carbonyl)-L-valyl-D-glutamate;
diethyl (3,5-dimethyl-1-phenyl-1H-pyrazole-4-carbonyl)-L-valyl-D-glutamate;
diethyl (1-(3,4-dichlorophenyl)-3,5-dimethyl-1H-pyrazole-4-carbonyl)-L-valyl-D-glutamate;
diethyl (1-(4-methoxyphenyl)-3,5-dimethyl-1H-pyrazole-4-carbonyl)-L-valyl-D-glutamate;
diethyl (1-(4-chlorophenyl)-3,5-dimethyl-1H-pyrazole-4-carbonyl)-L-valyl-D-glutamate;
diethyl (1-(1H-indol-6-yl)-3,5-dimethyl-1H-pyrazole-4-carbonyl)-L-valyl-D-glutamate;
diethyl (1-(6-aminopyridin-3-yl)-3,5-dimethyl-1H-pyrazole-4-carbonyl)-L-valyl-D-glutamate;
diethyl (1-(6-((tert-butoxycarbonyl)amino)pyridin-3-yl)-3,5-dimethyl-1H-pyrazole-4-carbonyl)-L-valyl-D-glutamate;
diethyl (1-(1H-indol-5-yl)-3,5-dimethyl-1H-pyrazole-4-carbonyl)-L-valyl-D-glutamate;
diethyl (3,5-dimethyl-1-(1H-pyrrolo[2,3-b]pyridin-5-yl)-1H-pyrazole-4-carbonyl)-L-valyl-D-glutamate;
diethyl (3,5-dimethyl-1-(2-oxo-1,2-dihydropyridin-4-yl)-1H-pyrazole-4-carbonyl)-L-valyl-D-glutamate;
diethyl (3,5-dimethyl-1-(pyridin-4-yl)-1H-pyrazole-4-carbonyl)-L-valyl-D-glutamate;
diethyl (3,5-dimethyl-1-(pyridin-2-yl)-1H-pyrazole-4-carbonyl)-L-valyl-D-glutamate;
diethyl (3,5-dimethyl-1-(pyridin-3-yl)-1H-pyrazole-4-carbonyl)-L-valyl-D-glutamate;
diethyl (3,5-dimethyl-1-(pyrimidin-5-yl)-1H-pyrazole-4-carbonyl)-L-valyl-D-glutamate;
diethyl (1-(2-methoxyphenyl)-3,5-dimethyl-1H-pyrazole-4-carbonyl)-L-valyl-D-glutamate;
diethyl (1-(3-methoxyphenyl)-3,5-dimethyl-1H-pyrazole-4-carbonyl)-L-valyl-D-glutamate;
diethyl (1-(pyridin-3-yl)-1H-pyrazole-4-carbonyl)-L-valyl-D-glutamate; and
diethyl(2-(3-(but-3-yn-1-yl)-3H-diazirin-3-yl) acetyl)-L-valyl-D-glutamate.

Embodiment 55. A compound having the formula diethyl (3,5-dimethyl-1-(2-oxo-1,2-dihydropyridin-4-yl)-1H-pyrazole-4-carbonyl)-L-valyl-D-glutamate.

Embodiment 56. A pharmaceutical composition comprising the compound of any one of Embodiments 1-55, admixed with a pharmaceutically acceptable carrier, diluent, or excipient.

Embodiment 57. The pharmaceutical composition of Embodiment 56, further comprising one or more adjuvants, immunotherapeutic agents, or anti-infective compounds or compositions.

Embodiment 58. The pharmaceutical composition of Embodiment 57, wherein the one or more adjuvants, immunotherapeutic agents, or anti-infective compounds or compositions is IL-1β, TNFα, IL-12p70, or anti-PD-L1.

Embodiment 59. The pharmaceutical composition of Embodiment 57, wherein the one or more adjuvants, immunotherapeutic agents, or anti-infective compounds or compositions is an additional NOD2 agonist compound or composition.

Embodiment 60. The pharmaceutical composition of Embodiment 59, wherein the additional NOD2 agonist compound or composition includes, but is not limited to, MDP, dMDP analogues including CinGVE, MurNAc, L18-MDP, N-glycolyl-MDP, murabutide, mifamurtide, M-TriLYS, and CL429.

Embodiment 61. A method of activating NOD2, comprising administering to a subject infected with a NOD2-responsive infection, disease, or disorder a therapeutically effective amount of the compound of any one of Embodiments 1-55 or the pharmaceutical composition of Embodiments 56-60.

Embodiment 62. A method of preventing, ameliorating, or treating a NOD2-responsive infection, disease, or disorder, comprising administering to a subject in need thereof a therapeutically effective amount of the compound of any one of Embodiments 1-55 or the pharmaceutical composition of Embodiments 56-60.

Embodiment 63. The method of Embodiment 62, further comprising administration of one or more additional therapeutic compounds or compositions.

Embodiment 64. The method of Embodiment 63, wherein the one or more therapeutic compounds or compositions is an adjuvant, anti-infective, immunotherapeutic, or anti-cancer therapeutic compound or composition.

Embodiment 65. The method of Embodiment 64, wherein at least one of the one or more therapeutic compounds or compositions is an immunotherapeutic compound or composition.

Embodiment 66. The method of Embodiment 63, wherein at least one of the one or more therapeutic compounds or compositions is an anti-cancer therapeutic compound or composition.

Embodiment 67. Use of the compound of any one of Embodiments 1-55 as an adjuvant in immunotherapy.

Embodiment 68. Use of the compound of any one of Embodiments 1-55 as an anti-infective agent.

Embodiment 69. Use of the compound of any one of Embodiments 1-55 as an anti-cancer agent.

Embodiment 70. The use of any one of Embodiments 67-69, wherein the compound is the compound of Embodiment 55.

Embodiment 71. Any compound, composition, method, or use as described herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “˜˜˜˜” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—□—C(—OH)═CH—), amide/imidic acid (—C(═O)—NH—□—C(—OH)═N—) and amidine (—C(═NR)—NH—□—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe— or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂ (C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, C(═NH)O(C₁₋₆ alkyl), —OC(═NH)(C₁₋₆ alkyl), —OC(═NH) OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal Rad substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

As used herein, the term “adjuvant” refers to a compound or composition that enhances a medical treatment, such as an added pharmacological agent added to a drug to increase or aid its effect, such as an immunological agent that increases an antigenic response or otherwise contributes to or enhances an existing medical regimen (for example, Freund's adjuvant).

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

In the various embodiments described herein, the NOD2 agonists of Formulae I-VI is a compound selected from the compounds shown in Table 1 below. The first two compounds, CinGVE and MDP, are reference compounds. TT004, TT005, TT006 are racemic mixtures of the head groups. The nomenclature was generated using ChemDraw. Assay data represents calculated pEC₅₀ (μM) values for dose-dependent activation of NOD2-expressing HEK-293T cells.

TABLE 1
NOD2 Agonist Structures and NOD2 Activation Assay Data

| | | | pEC₅₀ |
|---|---|---|---|
| Name | Structure | Nomenclature | (μM) |
| CinGVE | 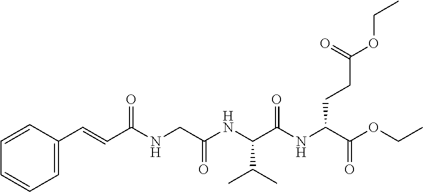 [SMILES: CCOC(=O)CC[C@@H](NC(=O)[C@@H](NC(=O)CNC(=O)/C=C/c1ccccc1)C(C)C)C(=O)OCC] | diethyl cinnamoylglycyl- L-valyl-D- glutamate | 5.9 |
| MDP | 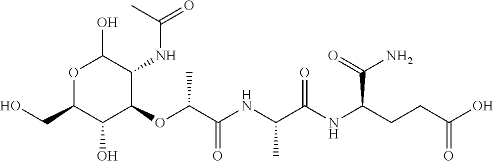 [SMILES: CC(=O)N[C@H]1C(O)O[C@H](CO)[C@@H](O)[C@@H]1O[C@H](C)C(=O)N[C@@H](C)C(=O)N[C@H](CCC(=O)O)C(N)=O] | (4R)-4-((2S)-2- ((2R)-2- (((3R,4R,5S,6R)- 3-acetamido-2,5- dihydroxy-6- (hydroxymethyl) tetrahydro-2H- pyran-4- yl)oxy)propanamido) propanamido)- 5-amino-5- oxopentanoic acid | 6.9 |
| TT001 | 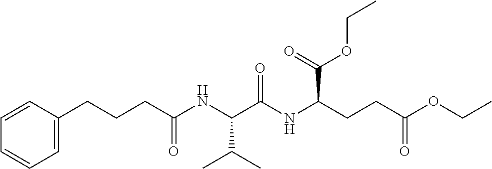 [SMILES: CCOC(=O)CC[C@@H](NC(=O)[C@@H](NC(=O)CCCc1ccccc1)C(C)C)C(=O)OCC] | diethyl (4- phenylbutanoyl)- L-valyl-D- glutamate | Active |
| TT002 | 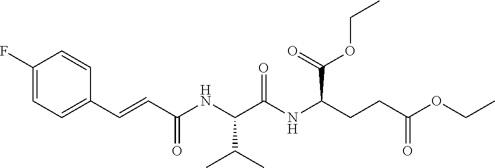 [SMILES: CCOC(=O)CC[C@@H](NC(=O)[C@@H](NC(=O)/C=C/c1ccc(F)cc1)C(C)C)C(=O)OCC] | diethyl ((E)-3-(4- fluorophenyl) acryloyl)-L-valyl- D-glutamate | Active |
| TT003 | 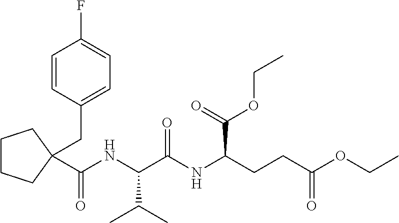 [SMILES: CCOC(=O)CC[C@@H](NC(=O)[C@@H](NC(=O)C1(Cc2ccc(F)cc2)CCCC1)C(C)C)C(=O)OCC] | diethyl (1-(4- fluorobenzyl) cyclopentane-1- carbonyl)-L- valyl-D- glutamate | 7.3 |
| TT004 | 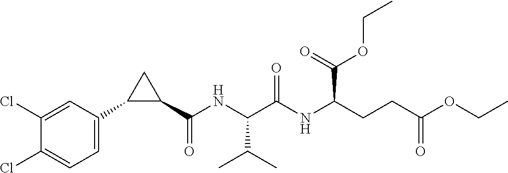 [SMILES: CCOC(=O)CC[C@@H](NC(=O)[C@@H](NC(=O)[C@@H]1C[C@H]1c1ccc(Cl)c(Cl)c1)C(C)C)C(=O)OCC] | rac-diethyl ((1R,2R)-2-(3,4- dichlorophenyl) cyclopropane-1- carbonyl)-L- valyl-D- glutamate | Active |
| TT005 | 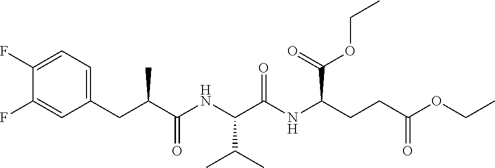 [SMILES: CCOC(=O)CC[C@@H](NC(=O)[C@@H](NC(=O)[C@H](C)Cc1ccc(F)c(F)c1)C(C)C)C(=O)OCC] | rac-diethyl ((R)- 3-(3,4- difluorophenyl)- 2- methylpropanoyl)- L-valyl-D- glutamate | 6.2 |
| TT007 | 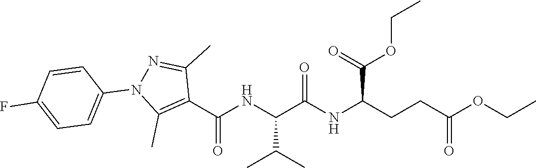 [SMILES: CCOC(=O)CC[C@@H](NC(=O)[C@@H](NC(=O)c1c(C)nn(-c2ccc(F)cc2)c1C)C(C)C)C(=O)OCC] | diethyl (1-(4- fluorophenyl)- 3,5-dimethyl- 1H-pyrazole-4- carbonyl)-L- valyl-D- glutamate | 6.0 |
| TT010 | 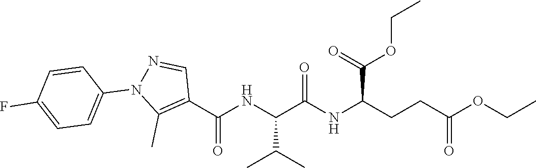 [SMILES: CCOC(=O)CC[C@@H](NC(=O)[C@@H](NC(=O)c1cnn(-c2ccc(F)cc2)c1C)C(C)C)C(=O)OCC] | diethyl (1-(4- fluorophenyl)-5- methyl-1H- pyrazole-4- carbonyl)-L- valyl-D- glutamate | 4.4 |
| TT011 | 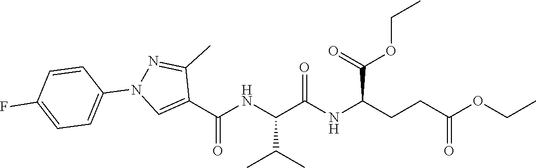 [SMILES: CCOC(=O)CC[C@@H](NC(=O)[C@@H](NC(=O)c1cn(-c2ccc(F)cc2)nc1C)C(C)C)C(=O)OCC] | diethyl (1-(4- fluorophenyl)-3- methyl-1H- pyrazole-4- carbonyl)-L- valyl-D- glutamate | 5.0 |
| TT012 | 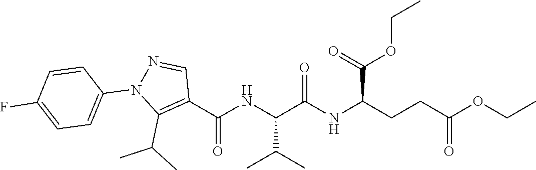 [SMILES: CCOC(=O)CC[C@@H](NC(=O)[C@@H](NC(=O)c1cnn(-c2ccc(F)cc2)c1C(C)C)C(C)C)C(=O)OCC] | diethyl (1-(4- fluorophenyl)-5- isopropyl-1H- pyrazole-4- carbonyl)-L- valyl-D- glutamate | 4.5 |
| TT013 | 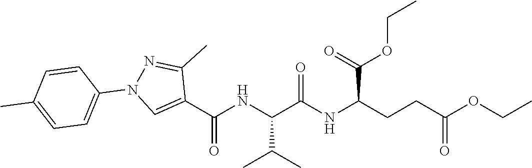 [SMILES: CCOC(=O)CC[C@@H](NC(=O)[C@@H](NC(=O)c1cn(-c2ccc(C)cc2)nc1C)C(C)C)C(=O)OCC] | diethyl (3- methyl-1-(p- tolyl)-1H- pyrazole-4- carbonyl)-L- valyl-D- glutamate | 5.0 |
| TT014 | 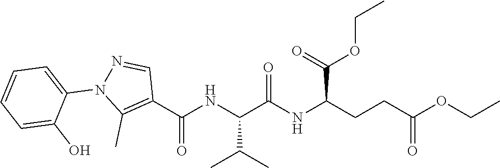 [SMILES: CCOC(=O)CC[C@@H](NC(=O)[C@@H](NC(=O)c1cnn(-c2ccccc2O)c1C)C(C)C)C(=O)OCC] | diethyl (1-(2- hydroxyphenyl)- 5-methyl-1H- pyrazole-4- carbonyl)-L- valyl-D- glutamate | 4.5 |
| TT016 | 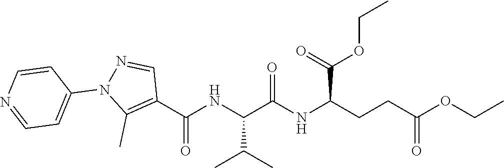 [SMILES: CCOC(=O)CC[C@@H](NC(=O)[C@@H](NC(=O)c1cnn(-c2ccncc2)c1C)C(C)C)C(=O)OCC] | diethyl (5- methyl-1- (pyridin-4-yl)- 1H-pyrazole-4- carbonyl)-L- valyl-D- glutamate | 4.1 |
| TT018 | 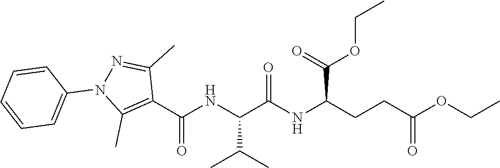 [SMILES: CCOC(=O)CC[C@@H](NC(=O)[C@@H](NC(=O)c1c(C)nn(-c2ccccc2)c1C)C(C)C)C(=O)OCC] | diethyl (3,5- dimethyl-1- phenyl-1H- pyrazole-4- carbonyl)-L- valyl-D- glutamate | 5.6 |
| TT022 | 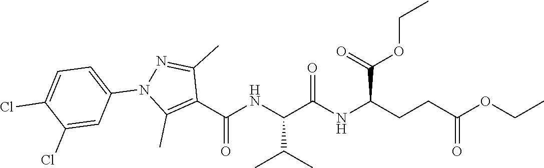 [SMILES: CCOC(=O)CC[C@@H](NC(=O)[C@@H](NC(=O)c1c(C)nn(-c2ccc(Cl)c(Cl)c2)c1C)C(C)C)C(=O)OCC] | diethyl (1-(3,4- dichlorophenyl)- 3,5-dimethyl- 1H-pyrazole-4- carbonyl)-L- valyl-D- glutamate | 5.9 |
| TT023 | 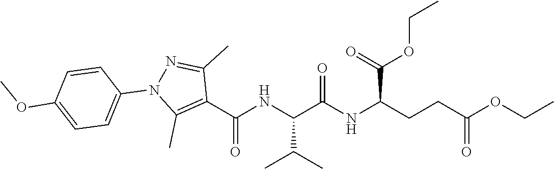 [SMILES: CCOC(=O)CC[C@@H](NC(=O)[C@@H](NC(=O)c1c(C)nn(-c2ccc(OC)cc2)c1C)C(C)C)C(=O)OCC] | diethyl (1-(4- methoxyphenyl)- 3,5-dimethyl- 1H-pyrazole-4- carbonyl)-L- valyl-D- glutamate | 5.7 |
| TT024 | 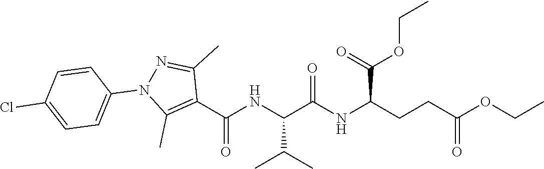 [SMILES: CCOC(=O)CC[C@@H](NC(=O)[C@@H](NC(=O)c1c(C)nn(-c2ccc(Cl)cc2)c1C)C(C)C)C(=O)OCC] | diethyl (1-(4- chlorophenyl)- 3,5-dimethyl- 1H-pyrazole-4- carbonyl)-L- valyl-D- glutamate | 6.0 |
| TT025 | 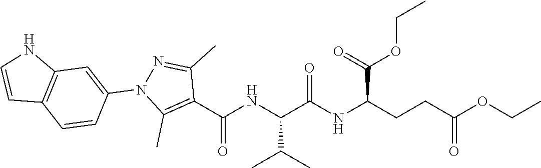 [SMILES: CCOC(=O)CC[C@@H](NC(=O)[C@@H](NC(=O)c1c(C)nn(-c2ccc3cc[nH]c3c2)c1C)C(C)C)C(=O)OCC] | diethyl (1-(1H- indol-6-yl)-3,5- dimethyl-1H- pyrazole-4- carbonyl)-L- valyl-D- glutamate | 6.8 |
| TT026 | 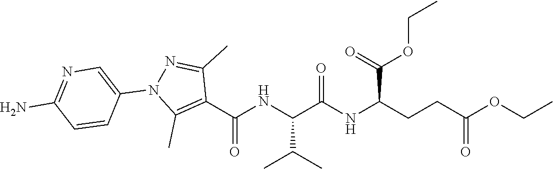 [SMILES: CCOC(=O)CC[C@@H](NC(=O)[C@@H](NC(=O)c1c(C)nn(-c2ccc(N)nc2)c1C)C(C)C)C(=O)OCC] | diethyl (1-(6- aminopyridin-3- yl)-3,5-dimethyl- 1H-pyrazole-4- carbonyl)-L- valyl-D- glutamate | 6.3 |
| TT027 | 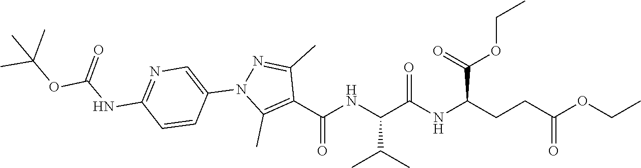 [SMILES: CCOC(=O)CC[C@@H](NC(=O)[C@@H](NC(=O)c1c(C)nn(-c2ccc(NC(=O)OC(C)(C)C)nc2)c1C)C(C)C)C(=O)OCC] | diethyl (1-(6- ((tert- butoxycarbonyl) amino)pyridin-3- yl)-3,5-dimethyl- 1H-pyrazole-4- carbonyl)-L- valyl-D- glutamate | 6.8 |
| TT028 | 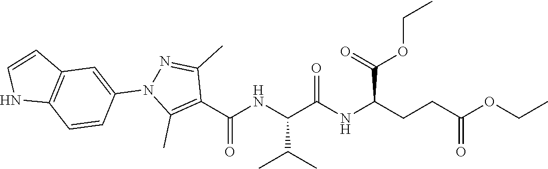 [SMILES: CCOC(=O)CC[C@@H](NC(=O)[C@@H](NC(=O)c1c(C)nn(-c2ccc3[nH]ccc3c2)c1C)C(C)C)C(=O)OCC] | diethyl (1-(1H- indol-5-yl)-3,5- dimethyl-1H- pyrazole-4- carbonyl)-L- valyl-D- glutamate | 6.6 |
| TT029 | 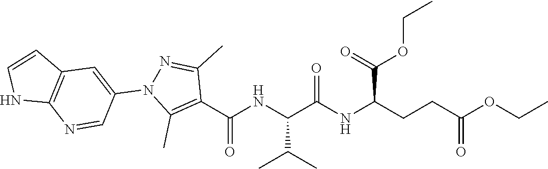 [SMILES: CCOC(=O)CC[C@@H](NC(=O)[C@@H](NC(=O)c1c(C)nn(-c2cnc3[nH]ccc3c2)c1C)C(C)C)C(=O)OCC] | diethyl (3,5- dimethyl-1-(1H- pyrrolo [2,3- b]pyridin-5-yl)- 1H-pyrazole-4- carbonyl)-L- valyl-D- glutamate | 6.4 |
| TT030 | 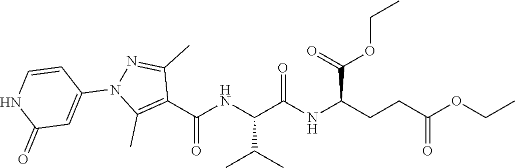 [SMILES: CCOC(=O)CC[C@@H](NC(=O)[C@@H](NC(=O)c1c(C)nn(-c2cc[nH]c(=O)c2)c1C)C(C)C)C(=O)OCC] | diethyl (3,5- dimethyl-1-(2- oxo-1,2- dihydropyridin- 4-yl)-1H- pyrazole-4- carbonyl)-L- valyl-D- glutamate | 6.6 |
| TT031 | 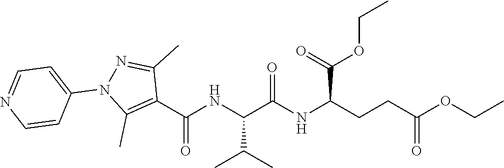 [SMILES: CCOC(=O)CC[C@@H](NC(=O)[C@@H](NC(=O)c1c(C)nn(-c2ccncc2)c1C)C(C)C)C(=O)OCC] | diethyl (3,5- dimethyl-1- (pyridin-4-yl)- 1H-pyrazole-4- carbonyl)-L- valyl-D- glutamate | 5.5 |
| TT032 | 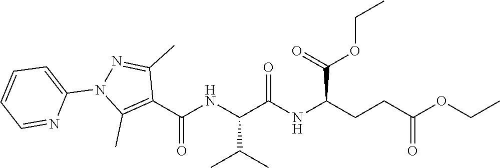 [SMILES: CCOC(=O)CC[C@@H](NC(=O)[C@@H](NC(=O)c1c(C)nn(-c2ccccn2)c1C)C(C)C)C(=O)OCC] | diethyl (3,5- dimethyl-1- (pyridin-2-yl)- 1H-pyrazole-4- carbonyl)-L- valyl-D- glutamate | 5.8 |
| TT033 | 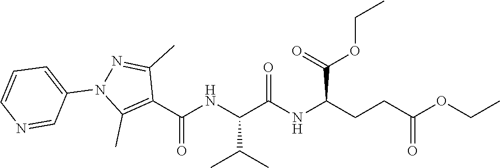 [SMILES: CCOC(=O)CC[C@@H](NC(=O)[C@@H](NC(=O)c1c(C)nn(-c2cccnc2)c1C)C(C)C)C(=O)OCC] | diethyl (3,5- dimethyl-1- (pyridin-3-yl)- 1H-pyrazole-4- carbonyl)-L- valyl-D- glutamate | 5.6 |
| TT034 | 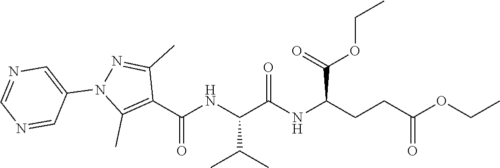 [SMILES: CCOC(=O)CC[C@@H](NC(=O)[C@@H](NC(=O)c1c(C)nn(-c2cncnc2)c1C)C(C)C)C(=O)OCC] | diethyl (3,5- dimethyl-1- (pyrimidin-5-yl)- 1H-pyrazole-4- carbonyl)-L- valyl-D- glutamate | 5.6 |
| TT035 | 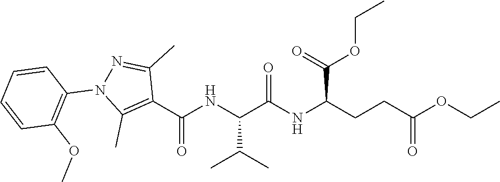 [SMILES: CCOC(=O)CC[C@@H](NC(=O)[C@@H](NC(=O)c1c(C)nn(-c2ccccc2OC)c1C)C(C)C)C(=O)OCC] | diethyl (1-(2- methoxyphenyl)- 3,5-dimethyl- 1H-pyrazole-4- carbonyl)-L- valyl-D- glutamate | 5.8 |
| TT036 | 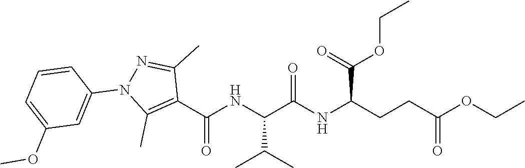 [SMILES: CCOC(=O)CC[C@@H](NC(=O)[C@@H](NC(=O)c1c(C)nn(-c2cccc(OC)c2)c1C)C(C)C)C(=O)OCC] | diethyl (1-(3- methoxyphenyl)- 3,5-dimethyl- 1H-pyrazole-4- carbonyl)-L- valyl-D- glutamate | 5.6 |
| TT037 | 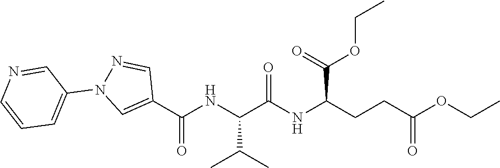 [SMILES: CCOC(=O)CC[C@@H](NC(=O)[C@@H](NC(=O)c1cnn(-c2cccnc2)c1)C(C)C)C(=O)OCC] | diethyl (1- (pyridin-3-yl)- 1H-pyrazole-4- carbonyl)-L- valyl-D- glutamate | 5.3 |
| x-alk-VE | 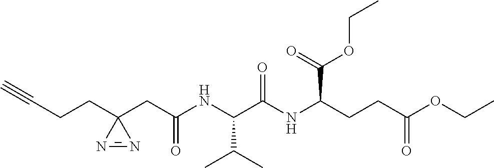 [SMILES: C#CCCC1(CC(=O)N[C@H](C(=O)N[C@H](CCC(=O)OCC)C(=O)OCC)C(C)C)N=N1] | diethyl (2-(3- (but-3-yn-1-yl)- 3H-diazirin-3- yl)acetyl)-L- valyl-D- glutamate | 5.8 |

### DETAILED DESCRIPTION OF THE FIGURES

FIG. 1(A-F). Virtual screening for small molecule NOD2 agonists. A, Docked structure of muramyl dipeptide (MDP) in the leucine-rich repeat (LRR) domain of rabbit NOD2 (pdb 5IRM). Inset shows amino acids within 5 angstroms of the docked MDP molecule. B, Results of virtual docking approach with reported chemical structures related to MDP. C, Workflow of virtual screening in which a library was generated using carboxylic acids appended to the L-Val-D-Glu dipeptide and then docked to the NOD2 LRR domain. D, Desmuramyl MDP (dMDP) structures resulting from the virtual screen that were synthesized for further screening. Chemicals shown as a pure enantiomer were synthesized as a racemic mixture. E, Activation of NOD2-expressing HEK-293T cells treated for 16 h with 10 UM of the indicated compound using a colorimetric assay. F, Dose-dependent activation of NOD2-expressing HEK-293T cells using the indicated compounds. Numbers in parentheses represent calculated negative log of the half maximal effective concentration (pEC₅₀) values. NOD activation assays were analyzed using a one-way ANOVA with Dunnett's multiple comparisons test compared to the DMSO control. pEC₅₀ values were derived by a three-parameter dose-response nonlinear regression. ****P<0.0001.

FIG. 2(A-G). Structure-activity relationship studies of 3,5-dimethyl-N-arylpyrazole NOD2 agonists. A, Structures of 3,5-dimethyl-N-arylpyrazole structures synthesized for SAR studies. B-F, Dose-dependent activation of NOD2-expressing HEK-293T cells using the indicated compounds grouped by similar structural perturbations. Numbers in parentheses represent calculated pEC₅₀ values. G, Graph of calculated pEC₅₀ values vs. calculated partition coefficient (clogP). Dotted lines show equivalent lipophilic ligand efficiency (LLE) values. Red arrow indicates optimization of the TT007 scaffold to TT030. pEC₅₀ values were derived by a three-parameter dose-response nonlinear regression.

FIG. 3(A-H). NOD2 agonist TT030 activates cytokine production in vitro and improves immune checkpoint inhibitor treatment of tumor growth in vivo. A-D, Measurements of secreted cytokine levels from human peripheral mononuclear blood cells (PMBCs) treated with the indicated compounds for 24 h without or with lipopolysaccharide (LPS). E, Structures of TT030 and its enantiomer (TT030-ent). F, Activation of NOD1- and NOD2-expressing HEK-293T cells with the indicated compounds. C₁₂-iE-DAP is a lipophilic version of the NOD1 agonist, iE-DAP. G, Growth profiles of B16-F10 subcutaneous tumors in wild-type C57BL/6 mice treated with 100 μg α-PD-L1 and 100 μg of the indicated compounds every other day starting at Day 7. H, Growth profiles of B16-F10 subcutaneous tumors in wild-type or Nod2^{-/-} C57BL/6 mice treated with 100 μg α-PD-L1 and 100 μg TT030 every other day starting at Day 7. Cytokine secretion assays and NOD activation assays were analyzed using a one-way ANOVA with Dunnett's multiple comparisons test compared to the DMSO control. Longitudinal tumor growth assays were analyzed using linear mixed-effects model analysis on the log transformed data. ns=not significant, *P<0.05, **P<0.01, ***P<0.001, ****P<0.0001.

FIG. 4(A-E). NOD2 agonist TT030 remodels intratumoral immune cell composition and elicits broad inflammatory signaling. A, Uniform manifold approximation and projection (UMAP) reduction of single intratumoral immune cells harvested from α-PD-L1 and TT030- and TT030-ent-treated tumors. B-C, Heatmap and quantification of the relative composition of intratumoral immune cell types divided by clusters. D, Quantification of the number of clusters that were enriched for individual Molecular Signature Database (MSigDB) hallmark gene sets. E, Bubble plot for enrichment of curated REACTOME gene sets involving NF-κB or interleukin 1 across cell clusters. For relative composition, absolute cell counts were analyzed by Pearson's chi-square test for count data with Holm's correction for multiple comparisons; exact P values are given. False discovery rates (FDRs) for gene set enrichment were obtained using model-based analysis of single transcriptomics (MAST). NOD activation assays were analyzed using a one-way ANOVA with Dunnett's multiple comparisons test compared to the DMSO control.

FIG. 5(A-B). Lead 3,5-dimethyl-N-arylpyrazole TT007 specifically activates NOD2. A-B, Activation of Null (control) and NOD1-expressing HEK-293T cells treated for 16 h with 10 μM of the indicated compound using a colorimetric assay. ****P<0.0001.

FIG. 6(A-E). Approach and synthesis of 3,5-dimethyl-N-arylpyrazole compound library. A, Diversification approach for compound library focusing on the N-aryl or pyrazole ring. B, Sample of library precursors generated with the Knorr pyrazole method. Numbers represent chemical yield. C, Sample of library precursors generated with the Chan-Evans-Lam method. Numbers represent chemical yield. D, Deprotection and coupling steps to produce library compounds. E, Dose-dependent activation of NOD2-expressing HEK-293T cells using the indicated compounds. Numbers in parentheses represent calculated pEC₅₀values. pEC₅₀ values were derived by a three-parameter dose-response nonlinear regression.

FIG. 7(A-B). NOD2 agonist TT030 activates cytokine production in vitro. A-B, Measurements of secreted cytokine levels from human peripheral mononuclear blood cells (PMBCs) treated with the indicated compounds for 24 h without or with lipopolysaccharide (LPS). Cytokine secretion assays and NOD activation assays were analyzed using a one-way ANOVA with Dunnett's multiple comparisons test compared to the DMSO control. *P<0.05, **P<0.01.

FIG. 8(A-B). Single cell RNA sequencing clustering and classification. A, Heat map of top 10 globally differentially expressed genes in each cluster (within cluster vs. other cells). Clusters are indicated by color, and representative genes are listed on the left. B, Heat map of individual genes used to classify gene clusters mapped onto a UMAP reduction of individual cells.

FIG. 9(A-B). TT030 upregulates cytotoxic T cell gene expression. A, Heat map of interferon gamma (Ifng) and granzyme B (Gzmb) expression mapped onto a UMAP reduction of individual cells. Red arrow indicates the increased cluster of CD8⁺ T cells. B, Quantile-quantile (QQ) plots of cytotoxic T cell genes Ifng and Gzmb. QQ plots were analyzed using a two-sided Wilcoxon rank-sum test; exact P values are given.

### Examples

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂-(mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMezSi (TBDMS), triethylamine (TEA or Et₃N), 2,2,6,6-tetramethylpiperidine 1-oxyl (TEMPO), triflate or CF₃SO₂-(Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford).

### General Synthetic Procedures.

All chemicals and solvents were purchased from Acros Organics, Alfa Aesar, Ambeed, Enamine, Sigma-Aldrich, TCI America and others and used without further purification.

HATU-mediated coupling. The corresponding carboxylic acid derivative (1.0-1.1 equiv.) and HATU (1.0-1.1 equiv.) were weighed in a scintillation vial and dissolved in DCM or DMF (0.2 M). The vial was flashed with argon gas and cooled to 0° C. To this was added DIPEA (5.0 equiv.), and the reaction mixture was stirred for 15 min. Then, a solution of the corresponding dipeptide (deprotected by acidolysis with 25% TFA/DCM or 4N HCl/1,4-dioxane) (1.0 equiv.) in DCM or DMF (0.2 M) was added, and the reaction mixture was allowed to warm to RT and stirred for 24-72 h. The reaction mixture was directly purified with flash chromatography when DCM was used as solvent or first concentrated by half before loading onto column when DMF was used as solvent. Alternatively, the reaction mixture was diluted with 70% EtOAc/hexanes, washed with aqueous NH₄Cl and brine, dried over MgSO₄, filtered, and concentrated before loading column. Flash chromatography was typically performed with a gradient of EtOAc/hexanes or MeOH/DCM.

Alkaline hydrolysis. The corresponding ester was weighed in a scintillation vial and dissolved in ethanol. The vial was flashed with argon gas. To a solution of ethyl ester in ethanol, was added 2 M LiOH (final concentration of LiOH: 1 M), and the reaction mixture was stirred at 50° C. for 24 h. Upon completion of the reaction, as monitored by TLC, the aqueous phase was acidified with 1 N HCl to PH ≈3 and extracted with EtOAc. The organic layer was dried over MgSO₄, filtered, and concentrated. Alternatively, the reaction mixture was acidified with Amberlite resin, filtered, and concentrated. The residue was dried under vacuum at 50° C.

Cell culture. The following cell lines were used and sourced as follows: HEK-Blue NOD2 (InvivoGen, hkb-hnod2), HEK-Blue NOD1 (InvivoGen, hkb-nod1), HEK-Blue Null2 (hkb-null2), and B16-F10 (ATCC CRL-6475). All cell lines were cultured at 37° C. in 5% CO₂ in DMEM (ThermoFisher, 11995065) supplemented with 10% fetal bovine serum (FBS), 100 U mL-1 penicillin, and 100 μg mL-1 streptomycin. Cells were maintained at no greater than 70% confluency and subcultured using TrypLE Express. Cell lines were routinely cultured without antibiotics to ensure no bacterial infection and tested for mycoplasma using a Universal Mycoplasma Detection Kit (ATCC 30-1012K). Primary human peripheral mononuclear blood cells (PBMCs) were cultured at 37° C. in 5% CO₂ in RPMI 1640 (ThermoFisher, 61870143) supplemented with 10% FBS. All experimental work using human samples was reviewed and approved by the Scripps Research Institutional Review Board (Protocol IRB-21-7816NOD2).

Animals. Specific pathogen-free, eight-week old male C₅₇BL/6J (B6, 000664) and B6.129S1—Nod2^{tm1Flv}/J (Nod2^{-/-}, 005763 from Flavell Lab, Yale School of Medicine) were obtained from The Jackson Laboratory. Animals were housed in autoclaved caging with SaniChip bedding and enrichment for nest building on a 12-hour light/dark cycle. Animals were provided gamma-irradiated chow (LabDiet, 5053) and sterile drinking water ad libitum. Animal care and experiments were conducted in accordance with NIH guidelines and approved by the Institutional Animal Care and Use Committee at Scripps Research (Protocol AUP-21-095).

### Synthesis and Chemical Characterization.

### Example 1. Diethyl D-Glutamate Hydrochloride

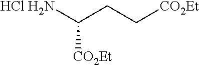

To EtOH (20 mL) was added acetyl chloride (2.1 mL, 0.03 mmol) on ice. After 30 min, D-glutamic acid (2.00 g, 0.014 mmol) was added in one portion, and the reaction mixture was refluxed for 4 h. The solvent was evaporated to provide crude material as white solid.
¹H NMR (DMSO-d₆, 600 MHz) δ 8.68 (br, 3H), 4.19 (q, 2 H, J=7.0 Hz), 4.07 (q, 2H J=7.0 Hz), 4.00 (m, 1H), 2.54 (m, 1H), 2.47 (m, 1H), 2.06 (m, 2H), 1.23 (t, 3H, J=7.1 Hz), 1.18 (t, 3H, J=7.1 Hz).
Example 2. Diethyl (tert-butoxycarbonyl)-L-valyl-D-glutamate

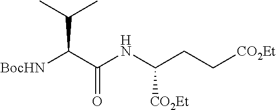 [SMILES: CCOC(=O)CC[C@@H](NC(=O)[C@@H](NC(=O)OC(C)(C)C)C(C)C)C(=O)OCC]

To a solution of Boc-L-valine-OH (906 mg, 4.17 mmol), diethyl D-glutamate hydrochloride (1000 mg, 4.17 mmol), EDC-HCl (500 mg, 4.17 mmol) and HOBt-xH₂O(620 mg, 4.59 mmol) in DMF (20 mL) was added TEA (1.2 mL, 8.34 mmol) at 0° C. The reaction mixture was allowed to warm to RT overnight. The reaction mixture was diluted with EtOAc, washed with aqueous NaHCO₃, aqueous NH₄Cl, and brine, dried over MgSO₄, filtered, and concentrated. The residue was purified by column chromatography (10% EtOAc/DCM). Yield: 914 mg (2.27 mmol, 54%), white powder.
¹H NMR (CDCl₃, 600 MHz) δ 6.67 (br, 1H), 4.97 (m, 1H), 4.59 (m, 1H), 4.20 (q, 2H, J=7.0 Hz), 4.14 (q, 2H, J=7.0 Hz), 4.00 (m 1H), 2.38 (m, 2H), 2.21 (m, 2H), 2.01 (m, 1H), 1.45 (s, 9H), 1.27 (m, 6H), 0.98 (d, 3H, J=6.3 Hz), 0.91 (d, 3H, J=6.5 Hz).
Example 3. Diethyl N-(tert-butoxycarbonyl)-N-methyl-L-valyl-D-glutamate

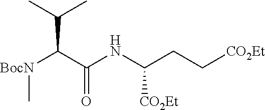 [SMILES: CCOC(=O)CC[C@@H](NC(=O)[C@H](C(C)C)N(C)C(=O)OC(C)(C)C)C(=O)OCC]

To a solution of Boc-N-methyl-L-valine-OH (386 mg, 1.67 mmol), diethyl D-glutamate hydrochloride (600 mg, 2.50 mmol), EDC-HCl (320 mg, 1.67 mmol) and HOBt-xH₂O (248 mg, 1.84 mmol) in DMF (10 mL) was added TEA (0.70 mL, 5.01 mmol) at 0° C. The reaction mixture was allowed to warm to RT overnight. The reaction mixture was diluted with EtOAc, washed with aqueous NaHCO₃, aqueous NH₄Cl, and brine, dried over MgSO₄, filtered, and concentrated. The residue was purified by flash chromatography using a gradient of EtOAc in hexanes. Yield: 388 mg (0.93 mmol, 56%), colourless oil.
¹H NMR (CDCl₃, 600 MHz) δ 6.77 (br, 1H), 4.51 (m, 1H), 4.13 (m, 4H), 2.76 (s, 3H), 2.36 (m, 2H), 2.24 (m, 2H), 1.96 (m, 1H), 1.48 (s, 9H), 1.25 (m, 6H), 0.91 (d, 3H, J=6.4 Hz), 0.87 (d, 3H, J=6.4 Hz).
Example 4. Diethyl (1-(4-fluorophenyl)-3,5-dimethyl-1H-pyrazole-4-carbonyl)-L-valyl-D-glutamate (TT007)

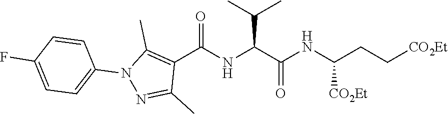 [SMILES: CCOC(=O)CC[C@@H](NC(=O)[C@@H](NC(=O)c1c(C)nn(-c2ccc(F)cc2)c1C)C(C)C)C(=O)OCC]

TT007 was synthesised according to the general procedure for HATU-mediated coupling. Yield: 35 mg (0.067 mmol, 75%).
¹H NMR (CDCl₃, 600 MHz) δ 7.36 (dd, 2H, J_{H,H}=8.9 Hz, 4J_{H,F}=4.8 Hz), 7.17 (dd, 2H, J_{H,H}=8.5 Hz, ³J_{H,F}=8.5 Hz), 6.72 (d, 1H, J=7.3 Hz), 6.29 (d, 1H, J=8.2 Hz), 4.57 (m, 2H), 4.20 (m, 2H), 4.13 (m, 2H), 2.53 (s, 3H), 2.48 (s, 3H), 2.46-1.94 (m, 5H), 1.26 (m, 6H), 1.05 (d, 3H, J=6.8 Hz), 1.02 (d, 3H, J=6.8 Hz).
¹³C NMR (CDCl₃, 150 MHz) δ 172.8 (s), 171.4 (s), 171.2 (s), 164.5 (s), 162.2 (d, ¹J_{C,F}=247 Hz), 147.5 (s), 142.5 (s), 135.0 (s), 127.5 (d, ³J_{C,F}=9 Hz), 116.2 (d, ²J_{C,F}=22 Hz), 114.4 (s), 61.7 (s), 60.8 (s), 57.9 (s), 52.0 (s), 31.3 (s), 30.3 (s), 27.1 (s), 19.5 (s), 17.9 (s), 14.21 (s), 14.15 (s), 14.12 (s), 12.2 (s).
HRMS m/z: [M+H]⁺ Calcd for C₂₆FH₃₆N₄O₆ 519.2613; Found 519.2624.
Example 5. Diethyl (1-phenyl-3-(m-tolyl)-1H-pyrazole-4-carbonyl)-L-valyl-D-glutamate (TT008)

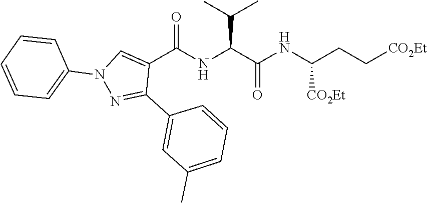 [SMILES: CCOC(=O)CC[C@@H](NC(=O)[C@@H](NC(=O)c1cn(-c2ccccc2)nc1-c1cccc(C)c1)C(C)C)C(=O)OCC]

TT008 was synthesised according to the general procedure for HATU-mediated coupling.

Yield: 34 mg (0.060 mmol, 67%).
¹H NMR (CDCl₃, 600 MHz) δ 8.54 (s, 1H), 7.75 (d, 2H, J=7.9 Hz), 7.49-7.29 (m, 7H), 6.76 (d, 1H, J=7.4 Hz), 6.13 (d, 1H, J=8.1 Hz), 4.53 (m, 1H), 4.39 (m, 1H), 4.18 (q, 2H, J=7.1 Hz), 4.08 (m, 2H), 2.42 (s, 3H), 2.40-2.00 (m, 5H), 1.26 (t, 3H, J=7.1 Hz), 1.21 (t, 3H, J=7.1 Hz), 0.84 (d, 3H, J=6.8 Hz), 0.61 (d, 3H, J=6.8 Hz).
¹³C NMR (CDCl₃, 150 MHz) δ 172.9, 171.5, 170.9, 162.9, 151.4, 139.3, 139.0, 132.1, 131.3, 130.3, 130.0, 129.6, 129.1, 127.3, 126.5, 119.4, 117.8, 61.6, 60.7, 58.4, 51.8, 30.3, 30.0, 27.1, 21.4, 19.4, 17.1, 14.1.

HRMS m/z: [M+H]⁺ Calcd for C₃₁H₃₉N₄O₆ 563.2864; Found 563.2878.

Example 6. Diethyl (1-(4-fluorophenyl)-3-(m-tolyl)-1H-pyrazole-4-carbonyl)-L-valyl-D-glutamate (TT009)

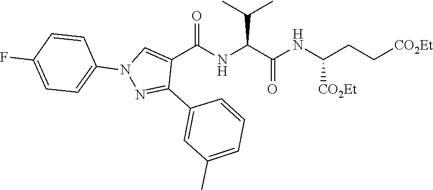 [SMILES: CCOC(=O)CC[C@@H](NC(=O)[C@@H](NC(=O)c1cn(-c2ccc(F)cc2)nc1-c1cccc(C)c1)C(C)C)C(=O)OCC]

TT009 was synthesised according to the general procedure for HATU-mediated coupling.
Yield: 44 mg (0.076 mmol, 84%).
¹H NMR (CDCl₃, 600 MHz) δ 8.47 (s, 1H), 7.72 (m, 2H), 7.49 (m, 2H), 7.41 (t, 1H, J=7.6 Hz), 7.30 (d, 1H, J=7.6 Hz), 7.17 (m, 2H), 6.74 (d, 1H, J=7.4 Hz), 6.13 (d, 1H, J=8.0 Hz), 4.53 (m, 1H), 4.38 (m, 1H), 4.18 (q, 2H, J=7.1 Hz), 4.09 (m, 2H), 2.42 (s, 3H), 2.39-2.00 (m, 5H), 1.26 (t, 3H, J=7.1 Hz), 1.22 (t, 3H, J=7.1 Hz), 0.84 (d, 3H, J=6.8 Hz), 0.61 (d, 3H, J=6.8 Hz).
¹³C NMR (CDCl₃, 150 MHz) δ 172.9 (s), 171.5 (s), 170.9 (s), 162.8 (s), 161.2 (d, ¹J_{C,F}=246 Hz), 151.4 (s), 139.0 (s), 135.7 (s), 131.9 (s), 131.3 (s), 130.3 (s), 129.9 (s), 129.1 (s), 126.4 (s), 121.3 (d, ³J_{C,F}=8 Hz), 117.9 (s), 116.4 (d, ²J_{C,F}=23 Hz), 61.6 (s), 60.7 (s), 58.4 (s), 51.8 (s), 30.3 (s), 30.1 (s), 27.1 (s), 21.4 (s), 19.3 (s), 17.1 (s), 14.1 (s), 14.1 (s).
HRMS m/z: [M+H]⁺ Calcd for C₃₁FH₃₈N₄O₆ 581.2770; Found 581.2783.
Example 7. Diethyl (1-(4-fluorobenzyl)cyclopentane-1-carbonyl)-L-valyl-D-glutamate (TT003)

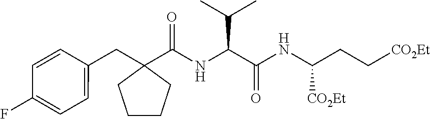 [SMILES: CCOC(=O)CC[C@@H](NC(=O)[C@@H](NC(=O)C1(Cc2ccc(F)cc2)CCCC1)C(C)C)C(=O)OCC]

TT003 was synthesised according to the general procedure for HATU-mediated coupling.
Yield: 36 mg (0.071 mmol, 71%).
HRMS m/z: [M+H]⁺ Calcd for C₂₇FH₄₀N₂O₆ 507.2865; Found 507.2877.
Example 8. Diethyl (3-(3,4-difluorophenyl)-2-methylpropanoyl)-L-valyl-D-glutamate (TT005)

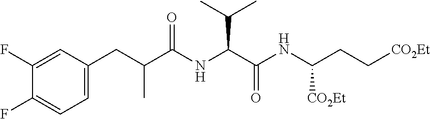 [SMILES: CCOC(=O)CC[C@@H](NC(=O)[C@@H](NC(=O)C(C)Cc1ccc(F)c(F)c1)C(C)C)C(=O)OCC]

TT005 (racemic mixture) was synthesised according to the general procedure for HATU-mediated coupling. Yield: 41 mg (0.085 mmol, 85%).

¹H NMR (CDCl₃, 600 MHz) δ 7.12-6.75 (m, 3H), 6.58 (br, 1H), 5.95 (br, 1H), 4.52 (m, 1H), 4.23 (m, 2H), 4.13 (m, 2H), 3.11-1.88 (m, 8H), 1.39-1.11 (m, 9H), 0.98-0.66 (m, 6H).
¹³C NMR (CDCl₃, 150 MHz) δ 175.4 & 175.1 (s), 172.6 (s), 171.3 (s), 171.2 (s), 150.1 (dd, ¹J_{C,F}=246 Hz, ²J_{C,F}=˜12 Hz), 149.0 (dd, ¹J_{C,F}=245 Hz, ²J_{C,F}=˜11 Hz), 136.7 & 136.6 (s), 124.9 (s), 117.7 (dd, ²J_{C,F}=15 Hz), 117.0 (d, ²J_{C,F}=17 Hz), 61.73 (s), 61.67 (s), 60.8 (s), 58.0 (s), 43.7 & 43.2 (s), 39.5 & 39.0 (s), 31.2 & 30.9 (s), 30.2 (s), 27.2 & 27.0 (s), [three of 19.2 (s), 18.9 (s), 17.9 (s), 17.82 (s), 17.79 (s), 17.7 (s)], 14.13 (s), 14.09 (s).
HRMS m/z: [M+H]⁺ Calcd for C₂₄F₂H₃₅N₂O₆ 485.2458; Found 485.2469.
Example 9. Diethyl(2-benzylbicyclo[2.2.1]heptane-2-carbonyl)-L-valyl-D-glutamate (TT006)

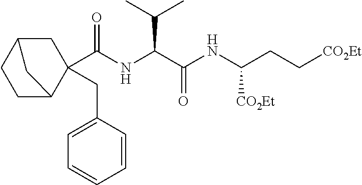 [SMILES: CCOC(=O)CC[C@@H](NC(=O)[C@@H](NC(=O)C1(Cc2ccccc2)CC2CCC1C2)C(C)C)C(=O)OCC]

TT006 (racemic mixture) was synthesised according to the general procedure for HATU-mediated coupling. Yield: 37 mg (0.072 mmol, 72%).
¹H NMR (CDCl₃, 600 MHz) δ 7.23-7.00 (m, 5H), 6.52 (br, 1H), 5.90 (br, 1H), 4.54 (m, 1H), 4.21 (m, 2H), 4.12 (q, 2H, J=7.1 Hz), 3.14 (m, 1H), 2.73 (m, 1H), 2.50-0.65 (m, 28H).
¹³C NMR (CDCl₃, 150 MHz) δ 176.1, 175.8, 172.6, 171.3, 170.84, 170.80, 137.7, 129.9, 129.6, 128.15, 128.11, 126.4, 126.3, 61.73, 61.66, 60.7, 58.7, 58.4, 56.4, 56.2, 51.70, 51.67, 46.5, 46.4, 45.9, 45.6, 38.6, 38.23, 38.18, 37.8, 37.64, 37.60, 37.5, 36.5, 31.4, 31.1, 30.5, 30.22, 30.18, 28.3, 28.2, 27.5, 27.4, 25.8, 25.7, 19.2, 18.3, 18.2, 14.2.
HRMS m/z: [M+H]⁺ Calcd for C₂₉H₄₃N₂O₆ 515.3116; Found 515.3130.
Example 10. Diethyl (rac-(1R,2R)-2-(3,4-dichlorophenyl)cyclopropane-1-carbonyl)-L-valyl-D-glutamate (TT004)

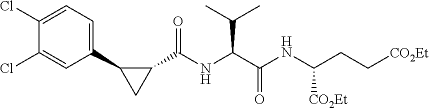 [SMILES: CCOC(=O)CC[C@@H](NC(=O)[C@@H](NC(=O)[C@@H]1C[C@H]1c1ccc(Cl)c(Cl)c1)C(C)C)C(=O)OCC]

TT004 (racemic mixture) was synthesised according to the general procedure for HATU-mediated coupling. Yield: 11 mg (0.021 mmol, 21%).
¹H NMR (CDCl₃, 600 MHz) δ 7.32 (m, 1H), 7.18 (m, 1H), 6.93 (m, 1H), 6.69 (m, 1H), 6.24 (m, 1H), 4.56 (m, 1H), 4.37 (m, 1H), 4.20 (m, 2H), 4.12 (m, 2H), 2.57-1.94 (m, 6H), 1.67 (m, 2H), 1.26 (m, 7H), 0.96 (m, 2H).
¹³C NMR (CDCl₃, 150 MHz) δ 172.8, 171.5, 171.4, 171.03, 170.98, 141.1, 141.0, 132.5, 132.4, 130.4, 130.3, 130.2, 128.4, 128.1, 125.7, 125.6, 61.8, 60.8, 58.5, 58.4, 51.9, 31.2, 30.9, 30.3, 27.1, 27.0, 26.54, 26.48, 24.3, 24.2, 19.3, 17.9, 16.4, 16.1, 14.1.
HRMS m/z: [M+H]⁺ Calcd for C₂₄Cl₂H₃₃N₂O₆ 515.1710; Found 515.1723.
Example 11. Diethyl ((E)-3-(4-fluorophenyl)acryloyl)-L-valyl-D-glutamate (TT002)

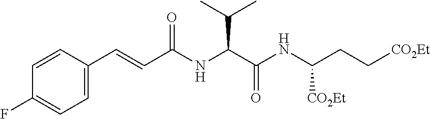 [SMILES: CCOC(=O)CC[C@@H](NC(=O)[C@@H](NC(=O)/C=C/c1ccc(F)cc1)C(C)C)C(=O)OCC]

To the solution of the dipeptide (0.10 mmol) and DIPEA (87 μL, 0.50 mmol) in DMF (1 mL) was added (4-fluorophenyl)acryloyl chloride (37 mg, 0.20 mmol) at 0° C. The reaction mixture was allowed to warm to RT over 4 h, quenched with NH₄Cl, extracted three times with DCM, dried over MgSO₄, filtered, and concentrated. The residue was purified with flash chromatography using a gradient of EtOAC in DCM. Yield: 26 mg (0.058 mmol, 58%).
¹H NMR (CDCl₃, 600 MHz) δ 7.60 (d, 1H, J=15.5 Hz), 7.49 (dd, 2H, J_{H,H}=8.3 Hz, ⁴J_{H,F}=5.5 Hz), 7.06 (dd, 2H, J_{H,H}=8.5 Hz, ³J_{H,F}=8.5 Hz), 6.81 (d, 1H, J=7.1 Hz), 6.38 (d, 1H, J=15.4 Hz), 6.19 (d, 1H, J=8.5 Hz), 4.56 (m, 1H), 4.49 (m, 1H), 4.20 (m, 2H), 4.13 (q, 2H, J=7.1 Hz), 2.40 (m, 2H), 2.21 (m, 2H), 2.04 (m, 1H), 1.26 (m, 6H), 1.01 (d, 3H, J=6.7 Hz), 0.98 (d, 3H, J=6.8 Hz).
HRMS m/z: [M+H]⁺ Calcd for C₂₃FH₃₂N₂O₆ 451.2239; Found 451.2251.
Example 12. Diethyl (4-phenylbutanoyl)-L-valyl-D-glutamate (TT001)

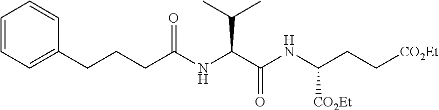 [SMILES: CCOC(=O)CC[C@@H](NC(=O)[C@@H](NC(=O)CCCc1ccccc1)C(C)C)C(=O)OCC]

TT001 was synthesised according to the general procedure for HATU-mediated coupling.
Yield: 25 mg (0.056 mmol, 56%).
¹H NMR (CDCl₃, 600 MHz) δ 7.28 (m, 2H), 7.18 (m, 3H), 6.70 (br, 1H), 5.96 (br, 1H), 4.54 (m, 1H), 4.35 (m, 1H), 4.18 (q, 2H, J=7.0 Hz), 4.12 (q, 2H, J=7.1 Hz), 2.65 (t, 2H, J=7.5 Hz), 2.50-1.90 (m, 9H), 1.26 (m, 6H), 0.96 (d, 3H, J=6.8 Hz), 0.93 (d, 3H, J=6.8 Hz).
HRMS m/z: [M+H]⁺ Calcd for C₂₄H₃₇N₂O₆ 449.2646; Found 449.2656.
Example 13. Diethyl (1-(2-methoxyphenyl)-5-methyl-1H-pyrazole-4-carbonyl)-L-valyl-D-glutamate (TT015)

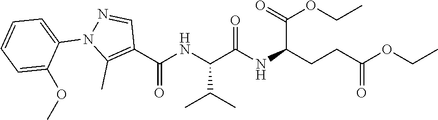 [SMILES: CCOC(=O)CC[C@@H](NC(=O)[C@@H](NC(=O)c1cnn(-c2ccccc2OC)c1C)C(C)C)C(=O)OCC]

TT015 was synthesised according to the general procedure for HATU-mediated coupling.
Yield: 66 mg (0.13 mmol, 71%).
¹H NMR (CDCl₃, 600 MHz) δ 7.90 (s, 1H), 7.45 (m, 1H), 7.30 (m, 1H), 7.06 (m, 2H), 7.78 (d, 1H, J=6.8 Hz), 6.32 (d, 1H, J=8.2 Hz), 4.59 (m, 1H), 4.53 (m, 1H), 4.20 (m, 2H), 4.13 (m, 2H), 3.80 (s, 3H), 2.42 (m, 2H), 2.38 (s, 3H), 2.28 (m, 1H), 2.22 (m, 1H), 2.06 (m, 1H), 1.26 (m, 6H), 1.04 (d, 3H, J=6.8 Hz), 1.02 (d, 3H, J=6.8 Hz).
¹³C NMR (CDCl₃, 150 MHz) δ 172.9, 171.5, 163.9, 154.6, 144.2, 138.7, 130.9, 129.0, 127.6, 121.0, 114.5, 112.1, 61.7, 60.8, 58.1, 55.8, 52.0, 31.3, 30.4, 27.1, 19.5, 18.1, 14.24, 14.18, 11.2.
HRMS m/z: [M+H]⁺ Calcd for C₂₆H₃₆N₄O₇ 517.2657; Found 517.2654.
Example 14. Diethyl (1-(4-methoxyphenyl)-3,5-dimethyl-1H-pyrazole-4-carbonyl)-L-valyl-D-glutamate (TT023)

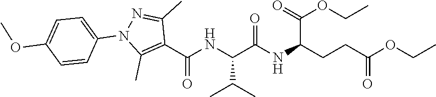 [SMILES: CCOC(=O)CC[C@@H](NC(=O)[C@@H](NC(=O)c1c(C)nn(-c2ccc(OC)cc2)c1C)C(C)C)C(=O)OCC]

TT023 was synthesised according to the general procedure for HATU-mediated coupling.
Yield: 63 mg (0.12 mmol, 66%).
¹H NMR (CDCl₃, 600 MHz) δ 7.28 (d, 2H, J=8.8 Hz), 6.97 (d, 2H, J=8.8 Hz), 6.74 (d, 1H, J=7.4 Hz), 6.27 (d, 1H, J=8.1 Hz), 4.57 (m, 2H), 4.20 (m, 2H), 4.12 (m, 2H), 3.85 (s, 3H), 2.53 (s, 3H), 2.46 (s, 3H), 2.44-1.95 (m, 5H), 1.26 (m, 6H), 1.05 (d, 3H, J=6.8 Hz), 1.02 (d, 3H, J=6.8 Hz).
¹³C NMR (CDCl₃, 150 MHz) δ 172.8, 171.5, 171.4, 164.8, 159.6, 147.2, 142.5, 131.9, 127.1, 114.4, 114.1, 61.8, 60.8, 58.0, 55.7, 52.0, 31.4, 30.4, 27.2, 19.6, 18.0, 14.3, 14.24, 14.20, 12.3.
HRMS m/z: [M+H]⁺ Calcd for C₂₇H₃₈N₄O₇ 531.2813; Found 531.2811.
Example 15. Diethyl (5-methyl-1-(pyridin-2-yl)-1H-pyrazole-4-carbonyl)-L-valyl-D-glutamate (TT017)

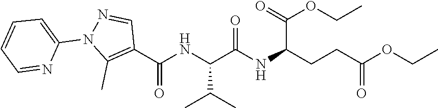 [SMILES: CCOC(=O)CC[C@@H](NC(=O)[C@@H](NC(=O)c1cnn(-c2ccccn2)c1C)C(C)C)C(=O)OCC]

TT017 was synthesised according to the general procedure for HATU-mediated coupling.
Yield: 27 mg (0.055 mmol, 92%).
¹H NMR (CDCl₃, 600 MHz) δ 8.50 (s, 1H), 7.89 (s, 1H), 7.86 (m, 1H), 7.80 (m, 1H), 7.29 (m, 1H), 6.79 (d, 1H, J=7.3 Hz), 6.34 (d, J=8.2 Hz), 4.59 (m, 1H), 4.53 (m, 1H), 4.20 (q, 2H, J=6.7 Hz), 4.12 (q, 2H, J=7.1 Hz), 2.90 (s, 3H), 2.50-2.00 (m, 5H), 1.26 (m, 6H), 1.04 (d, 3H, J=6.8 Hz), 1.02 (d, 3H, J=6.8 Hz).
¹³C NMR (CDCl₃, 150 MHz) δ 173.0, 171.6, 171.4, 163.7, 153.0, 148.0, 143.6, 139.4, 138.7, 122.6, 117.9, 116.8, 61.9, 60.9, 58.2, 52.1, 31.3, 30.4, 27.1, 19.5, 18.2, 14.3, 14.2, 13.1.
HRMS m/z: [M+H]⁺ Calcd for C₂₄H₃₃N₅O₆ 488.2504; Found 488.2502.
Example 16. Diethyl (5-methyl-1-(pyridin-4-yl)-1H-pyrazole-4-carbonyl)-L-valyl-D-glutamate (TT016)

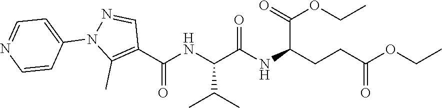 [SMILES: CCOC(=O)CC[C@@H](NC(=O)[C@@H](NC(=O)c1cnn(-c2ccncc2)c1C)C(C)C)C(=O)OCC]

TT016 was synthesised according to the general procedure for HATU-mediated coupling.

Yield: 12 mg (0.025 mmol, 41%).
¹H NMR (CDCl₃, 600 MHz) δ 8.76 (d, 2H, J=5.3 Hz), 7.93 (s, 1H), 7.46 (d, 2H, J=6.0 Hz), 6.76 (d, 1H, J=5.7 Hz), 6.40 (d, 1H, J=7.6 Hz), 4.58 (m, 1H), 4.53 (dd, 1H, J=8.3, 6.0 Hz), 4.20 (m, 2H), 4.13 (q, 2H, J=6.9 Hz), 2.41 (m, 2H), 2.24 (m, 2H), 2.06 (m, 1H), 1.27 (m, 6H), 1.04 (d, 3H, J=6.7 Hz), 1.02 (d, 3H, J=6.8 Hz).
¹³C NMR (CDCl₃, 150 MHz) δ 172.9, 171.4, 171.2, 163.1, 151.1, 145.8., 142.5, 139.7, 118.6, 116.9, 61.8, 60.8, 58.0, 52.0, 31.3, 30.3, 27.0, 19.4, 18.0, 14.2, 14.1, 12.3.
HRMS m/z: [M+H]⁺ Calcd for C₂₄H₃₃N₅O₆ 488.2504; Found 488.2500.
Example 17. Diethyl (1-(4-fluorophenyl)-5-isopropyl-1H-pyrazole-4-carbonyl)-L-valyl-D-glutamate (TT012)

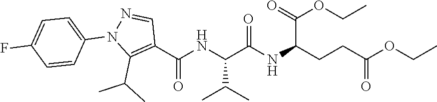 [SMILES: CCOC(=O)CC[C@@H](NC(=O)[C@@H](NC(=O)c1cnn(-c2ccc(F)cc2)c1C(C)C)C(C)C)C(=O)OCC]

TT012 was synthesised according to the general procedure for HATU-mediated coupling.
Yield: 20 mg (0.038 mmol, 63%).
¹H NMR (CDCl₃, 600 MHz) δ 7.82 (s, 1H), 7.34 (dd, 2H, J_{H,H}=8.8 Hz, ⁴J_{H,F}=4.7 Hz), 7.19 (dd, 2H, J_{H,H}=8.8 Hz, ³J_{H,F}=8.5 Hz), 6.78 (d, 1H, J=6.4 Hz), 6.37 (d, 1H, J=8.2 Hz), 4.59 (m, 1H), 4.51 (dd, 1H, J=8.4, 6.0 Hz), 4.20 (m, 2H), 4.13 (q, 2H, J=7.1 Hz), 3.22 (m, 1H), 2.41 (m, 2H), 2.27 (m, 1H), 2.21 (m, 1H), 2.05 (m, 1H), 1.33 (d, 6H, J=7.1 Hz), 1.27 (m, 6H), 1.04 (d, 3H, J=6.7 Hz), 1.02 (d, 3H, J=6.8 Hz).
¹³C NMR (CDCl₃, 150 MHz) δ 172.8 (s), 171.5 (s), 171.3 (s), 163.7 (s), 162.7 (d, ¹J_{C,F}=248 Hz), 151.5 (s), 139.2 (s), 135.7 (s), 128.6 (d, ³J_{C,F}=9 Hz), 116.2 (d, ²J_{C,F}=23 Hz), 114.8 (s), 61.7 (s), 60.8 (s), 58.3 (s), 51.9 (s), 31.1 (s), 30.3 (s), 27.1 (s), 26.3 (s), 20.7 (s), 20.7 (s), 19.4 (s), 18.0 (s), 14.2 (s), 14.1 (s).
HRMS m/z: [M+H]⁺ Calcd for C₂₇H₃₇FN₄O₆ 533.2770; Found 533.2768.
Example 18. Diethyl (1-(4-fluorophenyl)-3-methyl-1H-pyrazole-4-carbonyl)-L-valyl-D-glutamate (TT011)

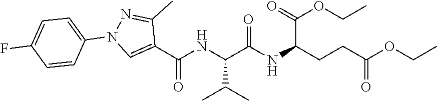 [SMILES: CCOC(=O)CC[C@@H](NC(=O)[C@@H](NC(=O)c1cn(-c2ccc(F)cc2)nc1C)C(C)C)C(=O)OCC]

TT011 was synthesised according to the general procedure for HATU-mediated coupling.
Yield: 29 mg (0.057 mmol, 96%).
¹H NMR (CDCl₃, 600 MHz) δ 8.23 (s, 1H), 7.63 (dd, 2H, J_{H,H}=8.9 Hz, ⁴J_{H,F}=4.6 Hz), 7.15 (dd, 2H, J_{H,H}=8.5 Hz, ³J_{H,F}=8.5 Hz), 6.82 (d, 1H, J=6.7 Hz), 6.42 (d, 1H, J=7.8 Hz), 4.56 (m, 2H), 4.19 (q, 2H, J=7.1 Hz), 4.11 (m, 2H), 2.58 (s, 3H), 2.40 (m, 2H), 2.22 (m, 2H), 2.06 (m, 1H), 1.25 (m, 6H), 1.04 (d, 3H, J=6.8 Hz), 1.01 (d, 3H, J=6.8 Hz).
¹³C NMR (CDCl₃, 150 MHz) δ 172.8 (s), 171.5 (s), 171.4 (s), 163.2 (s), 162.2 (d, ¹J_{C,F}=245 Hz), 149.8 (s), 135.7 (s), 129.1 (s), 121.1 (d, ³J_{C,F}=8 Hz), 117.5 (s), 116.4 (d, ²J_{C,F}=23 Hz), 61.7 (s), 60.8 (s), 58.1 (s), 52.0 (s), 31.3 (s), 30.3 (s), 27.0 (s), 19.4 (s), 18.1 (s), 14.14 (s), 14.09 (s), 13.8 (s).
HRMS m/z: [M+H]⁺ Calcd for C₂₅H₃₃FN₄O₆ 505.2457; Found 505.2457.
Example 19. Diethyl (1-(4-fluorophenyl)-5-methyl-1H-pyrazole-4-carbonyl)-L-valyl-D-glutamate (TT010)

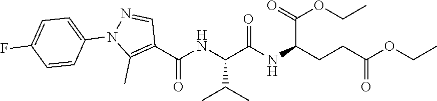 [SMILES: CCOC(=O)CC[C@@H](NC(=O)[C@@H](NC(=O)c1cnn(-c2ccc(F)cc2)c1C)C(C)C)C(=O)OCC]

TT010 was synthesised according to the general procedure for HATU-mediated coupling.
Yield: 30 mg (0.059 mmol, 99%).
¹H NMR (CDCl₃, 600 MHz) δ 7.87 (s, 1H), 7.39 (dd, 2H, J_{H,H}=8.8 Hz, ⁴J_{H,F}=4.8 Hz), 7.19 (dd, 2H, J_{H,H}=8.5 Hz, ³J_{H,F}=8.5 Hz), 6.77 (d, 1H, J=7.0 Hz), 6.35 (d, 1H, J=8.3 Hz), 4.58 (m, 1H), 4.52 (m, 1H), 4.20 (m, 2H), 4.13 (q, 2H, J=7.1 Hz), 2.55 (s, 3H), 2.41 (m, 2H), 2.24 (m, 2H), 2.05 (m, 1H), 1.26 (m, 6H), 1.04 (d, 3H, J=6.8 Hz), 1.02 (d, 3H, J=6.8 Hz).
¹³C NMR (CDCl₃, 150 MHz) δ 172.8 (s), 171.4 (s), 171.3 (s), 163.5 (s), 162.4 (d, ¹J_{C,F}=248 Hz), 142.3 (s), 138.6 (s), 135.0 (s) 127.4 (d, ³J_{C,F}=9 Hz), 116.2 (d, ²J_{C,F}=23 Hz), 115.4 (s), 61.7 (s), 60.8 (s), 58.0 (s), 52.0 (s), 31.3 (s), 30.3 (s), 27.1 (s), 19.4 (s), 18.0 (s), 14.2 (s), 14.1 (s), 11.8 (s).
HRMS m/z: [M+H]⁺ Calcd for C₂₅H₃₃FN₄O₆ 505.2457; Found 505.2455.
Example 20. Diethyl (3,5-dimethyl-1-phenyl-1H-pyrazole-4-carbonyl)-L-valyl-D-glutamate (TT018)

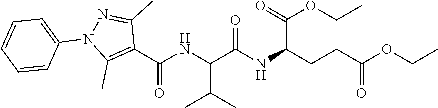 [SMILES: CCOC(=O)CC[C@@H](NC(=O)C(NC(=O)c1c(C)nn(-c2ccccc2)c1C)C(C)C)C(=O)OCC]

TT018 was synthesised according to the general procedure for HATU-mediated coupling.
Yield: 24 mg (0.048 mmol, 80%).
¹H NMR (CDCl₃, 600 MHz) δ 7.58-7.31 (m, 5H), 6.75 (br, 1H), 6.29 (d, 1H, J=8.0 Hz), 4.58 (m, 2H), 4.20 (m, 2H), 4.12 (m, 2H), 2.54 (s, 3H), 2.50 (s, 3H), 2.41 (m, 2H), 2.25 (m, 2H), 2.05 (m, 1H), 1.26 (m, 6H), 1.06 (d, 3H, J=6.8 Hz), 1.03 (d, 3H, J=6.9 Hz).
¹³C NMR (CDCl₃, 150 MHz) & 172.9, 171.5, 171.4, 164.7, 147.5, 142.5, 139.0, 129.3, 128.5, 125.7, 114.5, 61.8, 60.9, 58.1, 52.1, 31.4, 30.4, 27.2, 19.6, 18.1, 14.4, 14.3, 14.2, 12.5.
HRMS m/z: [M+H]⁺ Calcd for C₂₆H₃₆N₄O₆ 501.2708; Found 501.2703.
Example 21. Diethyl (1-(3,4-dichlorophenyl)-3,5-dimethyl-1H-pyrazole-4-carbonyl)-L-valyl-D-glutamate (TT022)

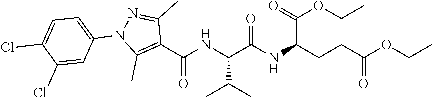 [SMILES: CCOC(=O)CC[C@@H](NC(=O)[C@@H](NC(=O)c1c(C)nn(-c2ccc(Cl)c(Cl)c2)c1C)C(C)C)C(=O)OCC]

TT022 was synthesised according to the general procedure for HATU-mediated coupling.
Yield: 25 mg (0.044 mmol, 73%).
¹H NMR (CDCl₃, 600 MHz) δ 7.56 (m, 2H), 6.71 (d, 1H, J=7.2 Hz), 6.30 (d, 1H, J=8.1 Hz), 4.57 (m, 2H), 4.20 (m, 2H), 4.13 (m, 2H), 2.53 (s, 3H), 2.52 (s, 3H), 2.40 (m, 2H), 2.25 (m, 2H), 2.06 (m, 1H), 1.26 (m, 6H), 1.05 (d, 3H, J=6.8 Hz), 1.02 (d, 3H, J=6.8 Hz).
¹³C NMR (CDCl₃, 150 MHz) δ 172.8, 171.4, 171.2, 164.2, 148.1, 142.4, 138.1, 133.3, 132.5, 130.8, 127.3, 124.4, 115.2, 61.8, 60.8, 57.9, 52.0, 31.4, 30.3, 27.1, 19.4, 17.9, 14.2, 14.1, 12.4.
HRMS m/z: [M+H]⁺ Calcd for C₂₆H₃₄Cl₂N₄O₆ 569.1928; Found 569.1928.
Example 22. Diethyl (1-(4-chlorophenyl)-3,5-dimethyl-1H-pyrazole-4-carbonyl)-L-valyl-D-glutamate (TT024)

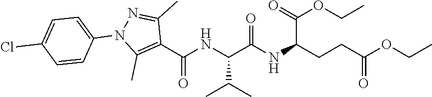 [SMILES: CCOC(=O)CC[C@@H](NC(=O)[C@@H](NC(=O)c1c(C)nn(-c2ccc(Cl)cc2)c1C)C(C)C)C(=O)OCC]

TT024 was synthesised according to the general procedure for HATU-mediated coupling.
Yield: 26 mg (0.049 mmol, 81%).
¹H NMR (CDCl₃, 600 MHz) δ 7.46 (d, 2H, J=8.6 Hz), 7.34 (d, 2H, J=8.6 Hz), 6.71 (d, 1H, J=7.1 Hz), 6.29 (d, 1H, J=8.0 Hz), 4.57 (m, 2H), 4.20 (m, 2H), 4.13 (m, 2H), 2.53 (s, 3H), 2.50 (s, 3H), 2.40 (m, 2H), 2.24 (m, 2H), 2.05 (m, 1H), 1.26 (m, 6H), 1.05 (d, 3H, J=6.8 Hz), 1.02 (d, 3H, J=6.8 Hz).
¹³C NMR (CDCl₃, 150 MHz) δ 172.9, 171.5, 171.3, 164.5, 147.8, 142.5, 137.5, 134.3, 129.5, 126.8, 114.9, 61.9, 60.9, 58.1, 52.1, 31.5, 30.4, 27.2, 19.6, 18.1, 14.33, 14.29, 14.2, 12.4.
HRMS m/z: [M+H]⁺ Calcd for C₂₆H₃₅ClN₄O₆ 535.2318; Found 535.2313.
Example 23. Diethyl (3-methyl-1-(p-tolyl)-1H-pyrazole-4-carbonyl)-L-valyl-D-glutamate (TT013)

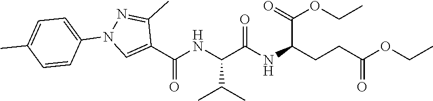 [SMILES: CCOC(=O)CC[C@@H](NC(=O)[C@@H](NC(=O)c1cn(-c2ccc(C)cc2)nc1C)C(C)C)C(=O)OCC]

TT013 was synthesised according to the general procedure for HATU-mediated coupling.
Yield: 22 mg (0.044 mmol, 73%).
¹H NMR (CDCl₃, 600 MHz) δ 7.87 (s, 1H), 7.28 (m, 4H), 6.77 (d, 1H, J=7.2 Hz), 6.32 (d, 1H, J=8.3 Hz), 4.58 (dd, 1H, J=12.7, 7.4 Hz), 4.52 (dd, 1H, J=7.8, 6.3 Hz), 4.20 (q, 2H, J=7.0 Hz), 4.12 (q, 2H, J=7.0 Hz), 2.55 (s, 3H), 2.42 (m, 5H), 2.24 (m, 2H), 2.06 (m, 1H), 1.26 (m, 6H), 1.04 (d, 3H, J=6.7 Hz), 1.02 (d, 3H, J=6.8 Hz).
¹³C NMR (CDCl₃, 150 MHz) δ 172.8, 171.4, 171.3, 163.7, 142.2, 138.7, 138.3, 136.4, 129.8, 125.3, 115.2, 61.7, 60.8, 58.0, 52.0, 31.2, 30.3, 27.1, 21.2, 19.4, 18.0, 14.2, 14.1, 11.9.
HRMS m/z: [M+H]⁺ Calcd for C₂₆H₃₆N₄O₆ 501.2708; Found 501.2707.
Example 24. Diethyl (1-(2-hydroxyphenyl)-5-methyl-1H-pyrazole-4-carbonyl)-L-valyl-D-glutamate (TT014)

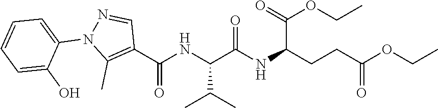 [SMILES: CCOC(=O)CC[C@@H](NC(=O)[C@@H](NC(=O)c1cnn(-c2ccccc2O)c1C)C(C)C)C(=O)OCC]

To a solution of TT015 (20 mg, 0.039 mmol) in DCM was added 1M BBr₃/DCM (78 μL, 0.078 mmol) on ice. The reaction mixture was stirred on ice for 2 h and then at RT for 1 h.
The reaction mixture was directly loaded on column and purified with flash chromatography using a gradient of MeOH in DCM. Yield: 19 mg (0.038 mmol, 98%).
¹H NMR (CDCl₃, 600 MHz) δ 8.61 (br, 1H), 7.96 (s, 1H), 7.29 (m, 1H), 7.20 (dd, 1H, J=7.9, 0.8 Hz), 7.10 (d, 1H, J=7.9 Hz), 7.02 (d, 1H, J=7.4 Hz), 6.97 (m, 1H), 6.59 (d, 1H, J=8.5 Hz), 4.57 (m, 1H), 4.53 (dd, 1H, J=8.3, 6.4 Hz), 4.18 (q, 2H, J=7.2 Hz), 4.11 (q, 2H, J=6.9 Hz), 2.60 (s, 3H), 2.40 (m, 2H), 2.22 (m, 2H), 2.04 (m, 1H), 1.25 (m, 6H), 1.02 (d, 3H, J=6.7 Hz), 1.00 (d, 3H, J=6.8 Hz).
¹³C NMR (CDCl₃, 150 MHz) δ 173.0, 171.5, 171.4, 163.3, 151.2, 143.4, 139.0, 130.0, 125.0, 120.0, 118.6, 115.7, 61.8, 60.9, 58.1, 52.0, 31.3, 30.3, 26.9, 19.4, 18.1, 14.19, 14.16, 12.2.
HRMS m/z: [M+H]⁺ Calcd for C₂₅H₃₄N₄O₇ 503.2500; Found 503.2499.
Example 25. Ethyl 1-(2-methoxyphenyl)-3,5-dimethyl-1H-pyrazole-4-carboxylate

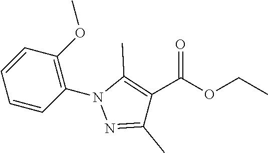 [SMILES: CCOC(=O)c1c(C)nn(-c2ccccc2OC)c1C]

A solution of 2-methoxyphenylhydrazine hydrochloride (349 mg, 2.00 mmol) and ethyl 2-acetyl-3-oxobutanoate (312 μL, 2.00 mmol) in ethanol (5 mL) was refluxed for 3 h. The solvent was evaporated, and the residue was purified with column chromatography eluting with 15% EtOAc/hexanes. Yield: 152 mg (0.55 mmol, 28%).
¹H NMR (CDCl₃, 600 MHz) δ 7.43 (m, 1H), 7.31 (dd, 1H, J=7.7, 1.4 Hz), 7.04 (m, 2H), 4.32 (q, 2H, J=7.1 Hz), 3.80 (s, 3H), 2.50 (s, 3H), 2.33 (s, 3H), 1.38 (t, 3H, J=7.1 Hz).
Example 26. Diethyl (1-(2-methoxyphenyl)-3,5-dimethyl-1H-pyrazole-4-carbonyl)-L-valyl-D-glutamate (TT035)

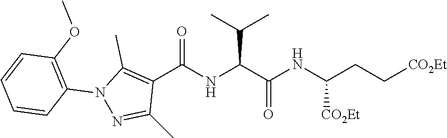 [SMILES: CCOC(=O)CC[C@@H](NC(=O)[C@@H](NC(=O)c1c(C)nn(-c2ccccc2OC)c1C)C(C)C)C(=O)OCC]

Ethyl 1-(2-methoxyphenyl)-3,5-dimethyl-1H-pyrazole-4-carboxylate (58 mg, 0.21 mmol) was hydrolysed according to the general procedure for alkaline hydrolysis [Intermediate yield: 45 mg (0.18 mmol, 87%)]. This material was used without further purification to synthesise TT035 according to the general procedure for HATU-mediated coupling. Yield: 29 mg (0.054 mmol, 30%).
¹H NMR (CDCl₃, 600 MHz) δ 7.48 (m, 1H), 7.34 (m, 1H), 7.07 (m, 2H), 6.73 (br, 1H), 6.35 (br, 1H), 4.58 (m, 2H), 4.20 (m, 2H), 4.13 (m, 2H), 3.83 (s, 3H), 2.81-1.89 (m, 11H), 1.27 (m, 6H), 1.06 (d, 3H, J=6.8 Hz), 1.03 (d, 3H, J=6.8 Hz).
¹³C NMR (CDCl₃, 150 MHz) δ 172.8, 171.43, 171.35, 164.7, 154.6, 147.5, 144.4, 130.8, 129.1, 127.3, 120.9, 113.3, 111.9, 61.7, 60.8, 58.0, 55.8, 52.0, 31.3, 30.3, 27.1, 19.5, 18.0, 14.3, 14.2, 14.1, 11.6.
HRMS m/z: [M+H]⁺ Calcd for C₂₇H₃₈N₄O₇ 531.2813; Found 531.2810.
Example 27. Ethyl 1-(3-methoxyphenyl)-3,5-dimethyl-1H-pyrazole-4-carboxylate

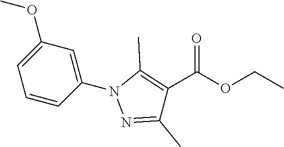 [SMILES: CCOC(=O)c1c(C)nn(-c2cccc(OC)c2)c1C]

A solution of 3-methoxyphenylhydrazine hydrochloride (87 mg, 0.5 mmol) and ethyl 2-acetyl-3-oxobutanoate (78 μL, 0.5 mmol) in ethanol (1 mL) was stirred at RT overnight. The solvent was evaporated, and the residue was purified with flash chromatography using a gradient of EtOAc in hexanes. Yield: 26 mg (0.094 mmol, 19%).
¹H NMR (CDCl₃, 600 MHz) δ 7.37 (m, 1H), 6.95 (m, 3H), 4.33 (q, 2H, J=7.1 Hz), 3.84 (s, 3H), 2.52 (s, 3H), 2.49 (s, 3H), 1.38 (t, 3H, J=7.1 Hz).
Example 28. Diethyl (1-(3-methoxyphenyl)-3,5-dimethyl-1H-pyrazole-4-carbonyl)-L-valyl-D-glutamate (TT036)

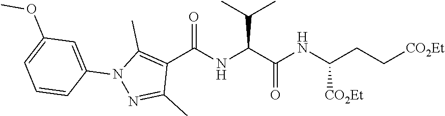 [SMILES: CCOC(=O)CC[C@@H](NC(=O)[C@@H](NC(=O)c1c(C)nn(-c2cccc(OC)c2)c1C)C(C)C)C(=O)OCC]

Ethyl 1-(3-methoxyphenyl)-3,5-dimethyl-1H-pyrazole-4-carboxylate (50 mg, 0.18 mmol) was hydrolysed according to the general procedure for alkaline hydrolysis [Intermediate yield: 33 mg (0.13 mmol, 71%)]. This material was used without further purification to synthesise TT036 according to the general procedure for HATU-mediated coupling. Yield: 22 mg (0.041 mmol, 32%).
¹H NMR (CDCl₃, 600 MHz) δ 7.39 (m, 1H), 6.98 (m, 3H), 6.74 (d, 1H, J=6.9 Hz), 6.34 (d, 1H, J=7.7 Hz), 4.58 (m, 2H), 4.20 (m, 2H), 4.13 (m, 2H), 3.86 (s, 3H), 2.60 (s, 3H), 2.52 (s, 3H), 2.41 (m, 2H), 2.25 (m, 2H), 2.06 (m, 1H), 1.27 (m, 6H), 1.06 (d, 3H, J=6.8 Hz), 1.02 (d, 3H, J=6.8 Hz).
¹³C NMR (CDCl₃, 150 MHz) δ 172.8, 171.4, 171.3, 164.5, 160.2, 147.3, 142.5, 139.7, 129.9, 117.8, 114.5, 114.4, 111.3, 61.7, 60.8, 58.0, 55.5, 52.0, 31.3, 30.3, 27.1, 19.5, 17.9, 14.2, 14.1, 12.4.
HRMS m/z: [M+H]⁺ Calcd for C₂₇H₃₈N₄O₇ 531.2813; Found 531.2810.
Example 29. Ethyl 3,5-dimethyl-1-(pyridin-2-yl)-1H-pyrazole-4-carboxylate

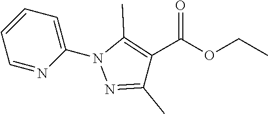 [SMILES: CCOC(=O)c1c(C)nn(-c2ccccn2)c1C]

A solution of 2-hydrazinylpyridine (55 mg, 0.5 mmol) and ethyl 2-acetyl-3-oxobutanoate (78 μL, 0.5 mmol) in acetic acid (0.5 mL) was stirred at RT overnight. The solvent was evaporated, and the residue was purified with flash chromatography using a gradient of EtOAc in hexanes. Yield: 109 mg (0.40 mmol, 79%).

¹H NMR (CDCl₃, 600 MHz) δ 8.49 (d, 1H, J=4.1 Hz), 7.83 (dd, 1H, J=7.1, 7.1 Hz), 7.76 (d, 1H, J=8.1 Hz), 7.26 (dd, 1H, J=6.1 Hz), 4.33 (q, 2H, J=7.1 Hz), 2.85 (s, 3H), 2.50 (s, 3H), 1.38 (t, 3H, J=7.1 Hz).
Example 30. Diethyl (3,5-dimethyl-1-(pyridin-2-yl)-1H-pyrazole-4-carbonyl)-L-valyl-D-glutamate (TT032)

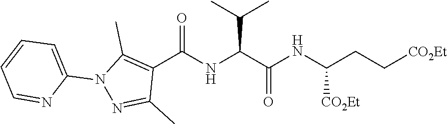 [SMILES: CCOC(=O)CC[C@@H](NC(=O)[C@@H](NC(=O)c1c(C)nn(-c2ccccn2)c1C)C(C)C)C(=O)OCC]

Ethyl 3,5-dimethyl-1-(pyridin-2-yl)-1H-pyrazole-4-carboxylate (109 mg, 0.40 mmol) was hydrolysed according to the general procedure for alkaline hydrolysis [Intermediate yield: 76 mg (0.37 mmol, 94%)]. This material was used without further purification to synthesise TT032 according to the general procedure for HATU-mediated coupling. Yield: 13 mg (0.026 mmol, 43%).
¹H NMR (CDCl₃, 600 MHz) δ 8.51 (m, 1H), 7.86 (m, 2H), 7.32 (m, 1H), 6.74 (br, 1H), 6.32 (br, 1H), 4.58 (m, 2H), 4.20 (m, 2H), 412 (q, 2H, J=7.1 Hz), 2.69-1.95 (m, 11H), 1.27 (m, 6H), 1.06 (d, 3H, J=6.9 Hz), 1.02 (d, 3H, J=6.8 Hz).
¹³C NMR (CDCl₃, 150 MHz) & 172.8, 171.4, 171.2, 164.5, 152.6, 148.4, 147.8, 143.1, 138.5, 122.1, 117.7, 116.2, 61.7, 60.8, 58.1, 52.0, 31.2, 30.3, 27.1, 19.5, 18.0, 14.2, 14.13, 14.09, 13.3.
HRMS m/z: [M+H]⁺ Calcd for C₂₅H₃₅N₅O₆ 502.2660; Found 502.2658.
Example 31. Ethyl 3,5-dimethyl-1-(pyridin-3-yl)-1H-pyrazole-4-carboxylate

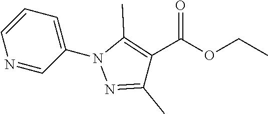 [SMILES: CCOC(=O)c1c(C)nn(-c2cccnc2)c1C]

A solution of 3-hydrazinylpyridine dihydrochloride (91 mg, 0.5 mmol), ethyl 2-acetyl-3-oxobutanoate (78 μL, 0.5 mmol), and triethylamine (140 μL, 1.0 mmol) in ethanol (1 mL) was stirred at RT for three days. The solvent was evaporated, and the residue was purified with flash chromatography using a gradient of EtOAc in hexanes. Yield: 25 mg (0.091 mmol, 18%).
¹H NMR (CDCl₃, 600 MHz) δ 8.70 (m, 2H), 7.80 (d, 1H), 7.46 (m, 1H), 4.34 (q, 2H, J=7.1 Hz), 2.56 (s, 3H), 2.50 (s, 3H), 1.39 (t, 3H, J=7.1 Hz).
Example 32. Diethyl (3,5-dimethyl-1-(pyridin-3-yl)-1H-pyrazole-4-carbonyl)-L-valyl-D-glutamate (TT033)

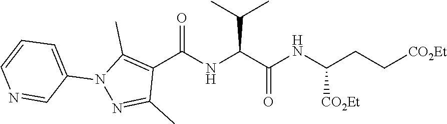 [SMILES: CCOC(=O)CC[C@@H](NC(=O)[C@@H](NC(=O)c1c(C)nn(-c2cccnc2)c1C)C(C)C)C(=O)OCC]

Ethyl 3,5-dimethyl-1-(pyridin-3-yl)-1H-pyrazole-4-carboxylate (25 mg, 0.091 mmol) was hydrolysed according to the general procedure for alkaline hydrolysis. Upon completion of the reaction, the aqueous phase was acidified with 1 N HCl to PH≈5 and extracted five times with EtOAc. The combined organic layers were dried over MgSO₄, filtered, and concentrated to afford the carboxylic acid derivative [Intermediate yield: 13 mg (0.06 mmol, 66%)]. This material was used without further purification to synthesise TT033 according to the general procedure for HATU-mediated coupling. Yield: 16 mg (0.032 mmol, 53%).
¹H NMR (CDCl₃, 600 MHz) δ 8.88 (br, 1H), 8.71 (br, 1H), 8.16 (br, 1H), 7.73 (br, 1H), 6.82 (d, 1H, J=7.3 Hz), 6.37 (d, 1H, J=8.2 Hz), 4.59 (m, 2H), 4.20 (m, 2H), 4.13 (q, 2H, 6.8 Hz), 2.61 (s, 3H), 2.53 (s, 3H), 2.42 (m, 2H), 2.24 (m, 3H), 2.06 (m, 1H), 1.27 (m, 6H), 1.06 (d, 3H, J=6.8 Hz), 1.02 (d, 3H, J=6.8 Hz).
¹³C NMR (CDCl₃, 150 MHz) δ 172.8, 171.4, 171.2, 164.1, 148.7, 148.0, 145.0, 142.7, 133.6, 129.5, 124.3, 115.6, 61.8, 60.8, 58.0, 52.0, 31.4, 30.3, 27.1, 19.5, 18.0, 14.2, 14.1, 14.0, 12.3.
HRMS m/z: [M+H]⁺ Calcd for C₂₅H₃₅N₅O₆ 502.2660; Found 502.2657.
Example 33. Ethyl 3,5-dimethyl-1-(pyridin-4-yl)-1H-pyrazole-4-carboxylate

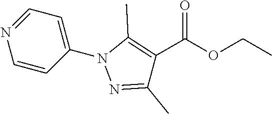 [SMILES: CCOC(=O)c1c(C)nn(-c2ccncc2)c1C]

A solution of 4-hydrazinylpyridine hydrochloride (73 mg, 0.5 mmol), ethyl 2-acetyl-3-oxobutanoate (78 μL, 0.5 mmol) in acetic acid (1 mL) was stirred at RT for three days. The solvent was evaporated, and the residue was purified with flash chromatography using a gradient of EtOAc in hexanes. Yield: 78 mg (0.28 mmol, 57%).
¹H NMR (CDCl₃, 600 MHz) δ 8.73 (d, 2H, J=5.9 Hz), 7.44 (d, 2H, J=6.1 Hz), 4.34 (q, 2H, J=7.1 Hz), 2.66 (s, 3H), 2.50 (s, 3H), 1.39 (t, 3H, J=7.1 Hz).
Example 34. Diethyl (3,5-dimethyl-1-(pyridin-4-yl)-1H-pyrazole-4-carbonyl)-L-valyl-D-glutamate (TT031)

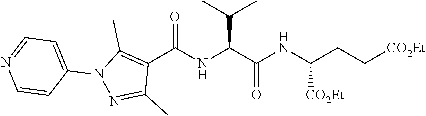 [SMILES: CCOC(=O)CC[C@@H](NC(=O)[C@@H](NC(=O)c1c(C)nn(-c2ccncc2)c1C)C(C)C)C(=O)OCC]

Ethyl 3,5-dimethyl-1-(pyridin-4-yl)-1H-pyrazole-4-carboxylate (78 mg, 0.28 mmol) was hydrolysed according to the general procedure for alkaline hydrolysis. Upon completion of the reaction, the aqueous phase was neutralised (pH≈6) with 1 N HCl resulting in precipitation. The precipitates were collected by filtration to afford the carboxylic acid derivative [Intermediate yield: 54 mg (0.25 mmol, 89%)]. This material was used without further purification to synthesise TT031 according to the general procedure for HATU-mediated coupling. Yield: 17 mg (0.034 mmol, 56%).
¹H NMR (CDCl₃, 600 MHz) δ 8.78 (d, 2H, J=5.6 Hz), 8.14 (d, 2H, J=5.8 Hz), 6.88 (br, 1H), 6.51 (d, 1H, J=5.3 Hz), 4.57 (m, 2H), 4.21 (q, 2H, J=7.0 Hz), 4.14 (q, 2H, J=7.0 Hz), 2.83 (s, 3H), 2.52 (s, 3H), 2.42 (m, 2H), 2.23 (m, 2H), 2.08 (m, 1H), 1.28 (m, 6H), 1.05 (d, 3H, J=6.8 Hz), 1.02 (d, 3H, J=6.8 Hz).
¹³C NMR (CDCl₃, 150 MHz) δ 172.8, 171.4, 171.1, 163.9, 149.7, 149.3, 146.7, 142.6, 118.3, 117.0, 61.8, 60.8, 58.0, 52.0, 31.5, 30.3, 27.0, 19.4, 18.0, 14.2, 14.1, 12.9.
HRMS m/z: [M+H]⁺ Calcd for C₂₅H₃₅N₅O₆ 502.2660; Found 502.2657.
Example 35. Ethyl 3,5-dimethyl-1-(pyrimidin-5-yl)-1H-pyrazole-4-carboxylate

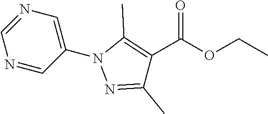 [SMILES: CCOC(=O)c1c(C)nn(-c2cncnc2)c1C]

Pyrimidin-5-ylboronic acid (62 mg, 0.50 mmol) and ethyl 3,5-dimethyl-1H-pyrazole-4-carboxylate (84 mg, 0.50 mmol) were weighed in a scintillation vial and dissolved in DMF (2 mL) under ambient atmosphere. To this Cu(OAc)₂ (68 mg, 0.38 mmol) and pyridine (81 μL, 1.0 mmol) were added. The reaction mixture was stirred at RT for three days and then diluted with 75% EtoAc in hexanes and washed with brine. Flash chromatography using a gradient of EtOAc in hexanes afforded the title compound. Yield: 24 mg (0.097 mmol, 19%).
¹H NMR (CDCl₃, 600 MHz) δ 9.22 (s, 1H), 8.88 (m, 2H), 4.32 (q, 2H, J=7.1 Hz), 2.59 (s, 3H), 2.48 (s, 3H), 1.37 (t, 3H, J=7.1 Hz).
Example 36. Diethyl (3,5-dimethyl-1-(pyrimidin-5-yl)-1H-pyrazole-4-carbonyl)-L-valyl-D-glutamate (TT034)

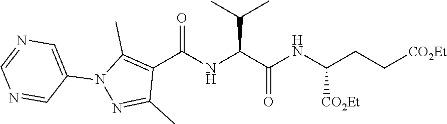 [SMILES: CCOC(=O)CC[C@@H](NC(=O)[C@@H](NC(=O)c1c(C)nn(-c2cncnc2)c1C)C(C)C)C(=O)OCC]

Ethyl 3,5-dimethyl-1-(pyrimidin-5-yl)-1H-pyrazole-4-carboxylate (24 mg, 0.097 mmol) was hydrolysed according to the general procedure for alkaline hydrolysis [Intermediate yield: 21 mg (0.10 mmol, quant.)]. This material was used without further purification to synthesise TT034 according to the general procedure for HATU-mediated coupling. Yield: 19 mg (0.038 mmol, 38%).
¹H NMR (CDCl₃, 600 MHz) δ 9.26 (s, 1H), 8.93 (m, 2H), 6.82 (br, 1H), 6.43 (br, 1H), 4.58 (m, 2H), 4.21 (m, 2H), 4.14 (m, 2H), 2.59 (s, 3H), 2.54 (s, 3H), 2.41 (m, 2H), 2.24 (m, 2H), 2.07 (m, 1H), 1.27 (m, 6H), 1.06 (d, 3H, J=6.7 Hz), 1.03 (d, 3H, J=6.8 Hz).
¹³C NMR (CDCl₃, 150 MHz) δ 172.8, 171.4, 171.2, 163.9, 157.1, 152.6, 149.4, 143.0, 134.3, 116.2, 61.8, 60.9, 58.1, 52.1, 31.5, 30.3, 27.0, 19.4, 18.0, 14.2, 14.1, 14.0, 12.2.
HRMS m/z: [M+H]⁺ Calcd for C₂₄H₃₄N₆O₆ 503.2613; Found 503.2611.
Example 37. Ethyl 3,5-dimethyl-1-(2-oxo-1,2-dihydropyridin-4-yl)-1H-pyrazole-4-carboxylate

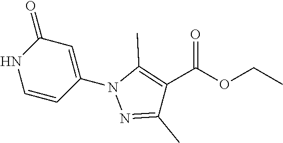 [SMILES: CCOC(=O)c1c(C)nn(-c2cc[nH]c(=O)c2)c1C]

A solution of 4-hydrazinylpyridin-2 (1H)-one (197 mg, 1.57 mmol), ethyl 2-acetyl-3-oxobutanoate (246 μL, 1.57 mmol) in acetic acid (3 mL) was stirred at RT overnight. The solvent was evaporated, and the residue was purified with column chromatography eluting with acetonitrile (two column volume) followed by 5% MeOH/DCM. Yield: 381 mg (1.45 mmol, 92%).
¹H NMR (CDCl₃, 600 MHz) δ 7.45 (d, 1H, J=7.1 Hz), 6.67 (d, 1H, J=7.1, 2.0 Hz), 6.57 (d, 1H, J=1.8 Hz), 4.34 (q, 2H, J=7.1 Hz), 2.69 (s, 3H), 2.48 (s, 3H), 1.38 (t, 3H, J=7.1 Hz).
Example 38. Diethyl (3,5-dimethyl-1-(2-oxo-1,2-dihydropyridin-4-yl)-1H-pyrazole-4-carbonyl)-L-valyl-D-glutamate (TT030)

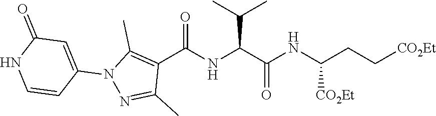 [SMILES: CCOC(=O)CC[C@@H](NC(=O)[C@@H](NC(=O)c1c(C)nn(-c2cc[nH]c(=O)c2)c1C)C(C)C)C(=O)OCC]

Ethyl 3,5-dimethyl-1-(2-oxo-1,2-dihydropyridin-4-yl)-1H-pyrazole-4-carboxylate (133 mg, 0.51 mmol) was hydrolysed according to the general procedure for alkaline hydrolysis. Upon completion of the reaction, the solvent was evaporated, and the residual aqueous phase was acidified with 1N HCl, which resulted in precipitation. The precipitates were collected by filtration and dried under vacuum at 60° C. [Intermediate yield: 119 mg (0.51 mmol, quant.)]. This material was used without further purification to synthesise TT030 according to the general procedure for HATU-mediated coupling. Upon completion of the reaction, reaction mixture was diluted with EtOAc, washed with aqueous NH₄Cl, dried over Na₂SO₄, filtered, and concentrated. The residue was purified with column chromatography using two different columns and eluents: first with 5% MeOH/DCM; then on a new column with 100% THF. Yield: 19 mg (0.037 mmol, 37%).
¹H NMR (CDCl₃, 600 MHz) δ 7.40 (d, 1H, J=7.1 Hz), 6.91 (d, 1H, J=7.1 Hz), 6.62 (m, 2H), 6.51 (d, 1H, J=7.1 Hz), 4.57 (m, 2H), 4.20 (m, 2H), 4.13 (q, 2H, J=7.1 Hz), 2.62 (s, 3H), 2.48 (s, 3H), 2.42 (m, 2H), 2.25 (m, 2H), 2.06 (m, 1H), 1.27 (m, 6H), 1.06 (d, 3H, J=6.8 Hz), 1.04 (d, 3H, J=6.8 Hz).
¹³C NMR (CDCl₃, 150 MHz) δ 172.8, 171.8, 171.6, 164.9, 164.3, 150.1, 149.2, 142.2, 135.4, 117.4, 111.6, 103.9, 61.8, 60.8, 58.4, 52.0, 31.2, 30.4, 27.0, 19.5, 18.2, 14.2, 14.1, 13.9, 13.1.
HRMS m/z: [M+H]⁺ Calcd for C₂₅H₃₅N₅O₇ 518.2609; Found 518.2606.
Example 39. Ethyl 1-(1H-indol-5-yl)-3,5-dimethyl-1H-pyrazole-4-carboxylate

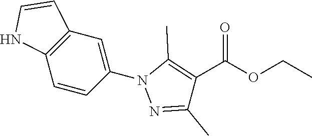 [SMILES: CCOC(=O)c1c(C)nn(-c2ccc3[nH]ccc3c2)c1C]

(1H-Indol-5-yl) boronic acid (64 mg, 0.40 mmol) and ethyl 3,5-dimethyl-1H-pyrazole-4-carboxylate (67 mg, 0.40 mmol) were weighed in a scintillation vial and dissolved in DMF (2 mL) under ambient atmosphere. To this Cu(OAc)₂ (54 mg, 0.30 mmol) and pyridine (64 μL, 0.80 mmol) were added. The reaction mixture was stirred at RT for three days and then diluted with 50% EtOAc in hexanes and washed with brine. Flash chromatography using a gradient of EtOAc in hexanes afforded the title compound. Yield: 76 mg (0.27 mmol, 67%). ¹H NMR (CDCl₃, 600 MHz) δ 8.39 (br, 1H), 7.61 (s, 1H), 7.44 (d, J=8.5 Hz), 7.30 (br, 1H), 7.17 (dd, J=8.5, 1.7 Hz), 6.61 (br, 1H), 4.34 (q, 2H, J=7.1 Hz), 2.52 (s, 3H), 2.49 (s, 3H), 1.39 (t, 3H, J=7.1 Hz).
Example 40. Diethyl (1-(1H-indol-5-yl)-3,5-dimethyl-1H-pyrazole-4-carbonyl)-L-valyl-D-glutamate (TT028)

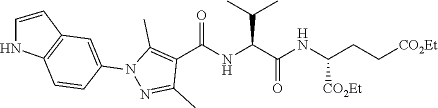 [SMILES: CCOC(=O)CC[C@@H](NC(=O)[C@@H](NC(=O)c1c(C)nn(-c2ccc3[nH]ccc3c2)c1C)C(C)C)C(=O)OCC]

Ethyl 1-(1H-indol-5-yl)-3,5-dimethyl-1H-pyrazole-4-carboxylate (76 mg, 0.27 mmol) was hydrolysed according to the general procedure for alkaline hydrolysis [Intermediate yield: 70 mg (0.27 mmol, quant.)]. This material was used without further purification to synthesise TT028 according to the general procedure for HATU-mediated coupling. Yield: 23 mg (0.043 mmol, 71%).
¹H NMR (CDCl₃, 600 MHz) δ 7.62 (s, 1H), 7.41 (br, 1H), 7.32 (br, 1H), 7.12 (br, 1H), 6.86 (br, 1H), 6.59 (br, 1H), 6.53 (br, 1H), 4.60 (m, 2H), 4.24 (q, 2H, J=6.7 Hz), 4.16 (q, 2H, J=6.9 Hz), 2.64 (s, 3H), 2.51 (s, 3H), 2.45 (m, 2H), 2.29 (m, 2H), 2.10 (m, 1H), 1.30 (m, 6H), 1.10 (d, 3H, J=6.7 Hz), 1.07 (d, 3H, J=6.8 Hz).
¹³C NMR (CDCl₃, 150 MHz) δ 172.8, 171.44, 171.41, 164.7, 146.7, 143.0, 135.5, 130.9, 127.9, 126.2, 119.9, 118.3, 113.7, 111.6, 103.1, 61.7, 60.8, 58.1, 52.0, 31.2, 30.3, 27.1, 19.5, 18.0, 14.2, 14.1, 12.3.
HRMS m/z: [M+H]⁺ Calcd for C₂₈H₃₇N₅O₆ 540.2817; Found 540.2813.
Example 41. Ethyl 1-(1H-indol-6-yl)-3,5-dimethyl-1H-pyrazole-4-carboxylate

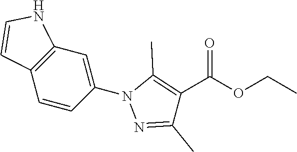 [SMILES: CCOC(=O)c1c(C)nn(-c2ccc3cc[nH]c3c2)c1C]

Indole-6-boronic acid (64 mg, 0.40 mmol) and ethyl 3,5-dimethyl-1H-pyrazole-4-carboxylate (67 mg, 0.40 mmol) were weighed in a scintillation vial and dissolved in DMF (2 mL) under ambient atmosphere. To this Cu(OAc)₂ (54 mg, 0.30 mmol) and pyridine (64 μL, 0.80 mmol) were added. The reaction mixture was stirred at RT for three days and then diluted with 50% EtOAc in hexanes and washed with brine. Flash chromatography using a gradient of EtOAc in hexanes afforded the title compound. Yield: 62 mg (0.22 mmol, 55%).
¹H NMR (CDCl₃, 600 MHz) δ 9.17 (br, 1H), 7.67 (br, 1H), 7.39 (br, 1H), 7.29 (br, 1H), 7.04 (br, 1H), 6.57 (br, 1H), 4.32 (q, 2H, J=7.0 Hz), 2.49 (s, 3H), 2.48 (s, 3H), 1.37 (t, 3H, J=7.1 Hz).
Example 42. Diethyl (1-(1H-indol-6-yl)-3,5-dimethyl-1H-pyrazole-4-carbonyl)-L-valyl-D-glutamate (TT025)

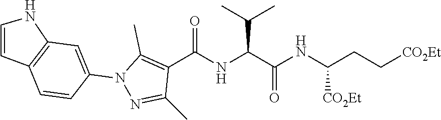 [SMILES: CCOC(=O)CC[C@@H](NC(=O)[C@@H](NC(=O)c1c(C)nn(-c2ccc3cc[nH]c3c2)c1C)C(C)C)C(=O)OCC]

Ethyl 1-(1H-indol-6-yl)-3,5-dimethyl-1H-pyrazole-4-carboxylate (62 mg, 0.22 mmol) was hydrolysed according to the general procedure for alkaline hydrolysis [Intermediate yield: 57 mg (0.22 mmol, quant.)]. This material was used without further purification to synthesise TT025 according to the general procedure for HATU-mediated coupling. Yield: 19 mg (0.035 mmol, 59%).
¹H NMR (CDCl₃, 600 MHz) δ 9.09 (br, 1H), 7.68 (m, 1H), 7.41 (br, 1H), 7.33 (br, 1H), 7.06 (dd, 1H, J=8.3, 1.4 Hz), 6.84 (d, 1H, J=7.3 Hz), 6.63 (br, 1H), 6.58 (s, 1H), 4.58 (m, 2H), 4.20 (q, 2H, J=7.1 Hz), 4.13 (q, 2H, J=6.8 Hz), 2.46 (s, 3H), 2.45 (s, 3H), 2.40 (m, 2H), 2.27 (m, 2H), 2.07 (m, 1H), 1.26 (m, 6H), 1.07 (d, 3H, J=6.7 Hz), 1.05 (d, 3H, J=6.8 Hz).
¹³C NMR (CDCl₃, 150 MHz) δ 172.8, 171.4, 171.3, 164.5, 146.9, 142.9, 135.4, 132.3, 128.2, 126.5, 121.0, 117.7, 114.2, 109.0, 102.6, 61.7, 60.8, 58.2, 52.0, 31.2, 30.3, 27.1, 19.5, 18.0, 14.2, 14.1, 13.8, 12.3.
HRMS m/z: [M+H]⁺ Calcd for C₂₈H₃₇N₅O₆ 540.2817; Found 540.2812.
Example 43. Ethyl 3,5-dimethyl-1-(1H-pyrrolo[2,3-b]pyridin-5-yl)-1H-pyrazole-4-carboxylate

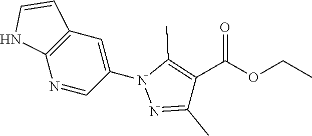 [SMILES: CCOC(=O)c1c(C)nn(-c2cnc3[nH]ccc3c2)c1C]

(1H-Pyrrolo[2,3-b]pyridin-5-yl) boronic acid (65 mg, 0.40 mmol) and ethyl 3,5-dimethyl-1H-pyrazole-4-carboxylate (67 mg, 0.40 mmol) were weighed in a scintillation vial and dissolved in DMF (2 mL) under ambient atmosphere. To this Cu(OAc)₂ (54 mg, 0.30 mmol) and pyridine (64 μL, 0.80 mmol) were added. The reaction mixture was stirred at RT for three days and then diluted with 50% EtOAc in hexanes and washed with brine. Flash chromatography using a gradient of EtOAc in hexanes afforded the title compound. Yield: 65 mg (0.23 mmol, 57%).
¹H NMR (CDCl₃, 600 MHz) δ 9.23 (br, 1H), 8.35 (s, 1H), 7.96 (s, 1H), 7.44 (d, 1H, J=2.3 Hz), 6.59 (dd, 1H, J=3.2, 2.0 Hz), 4.35 (q, 2H, J=7.1 Hz), 2.52 (s, 3H), 2.51 (s, 3H), 1.39 (t, 3H, J=7.1 Hz).
Example 44. Diethyl (3,5-dimethyl-1-(1H-pyrrolo[2,3-b]pyridin-5-yl)-1H-pyrazole-4-carbonyl)-L-valyl-D-glutamate (TT029)

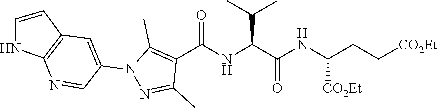 [SMILES: CCOC(=O)CC[C@@H](NC(=O)[C@@H](NC(=O)c1c(C)nn(-c2cnc3[nH]ccc3c2)c1C)C(C)C)C(=O)OCC]

Ethyl 3,5-dimethyl-1-(1H-pyrrolo[2,3-b]pyridin-5-yl)-1H-pyrazole-4-carboxylate (65 mg, 0.23 mmol) was hydrolysed according to the general procedure for alkaline hydrolysis [Intermediate yield: 26 mg (0.10 mmol, 44%)]. This material was used without further purification to synthesise TT029 according to the general procedure for HATU-mediated coupling. Yield: 16 mg (0.030 mmol, 30%).

¹H NMR (CDCl₃, 600 MHz) δ 8.49 (s, 1H), 8.40 (s, 1H), 7.69 (s, 1H), 7.07 (d, 1H, J=7.4 Hz), 6.81 (d, 1H, J=2.2 Hz), 6.59 (d, 1H, J=8.3 Hz), 4.67 (m, 1H), 4.62 (m, 1H), 4.22 (q, 2H, J=6.8 Hz), 4.16 (q, 2H, J=7.1 Hz), 2.55 (s, 3H), 2.53 (s, 3H), 2.46 (m, 2H), 2.29 (m, 2H), 2.11 (m, 1H), 1.29 (m, 6H), 1.09 (d, 3H, J=6.7 Hz), 1.07 (d, J=6.8 Hz).
¹³C NMR (CDCl₃, 150 MHz) δ 172.7, 171.8, 171.4, 164.9, 147.9, 146.6, 142.5, 138.9, 129.1, 127.9, 127.0, 120.6, 115.1, 101.6, 61.7, 60.8, 58.4, 52.1, 31.3, 30.3, 27.1, 19.5, 18.2, 14.2, 14.1, 13.9, 12.1.
HRMS m/z: [M+H]⁺ Calcd for C₂₇H₃₆N₆O₆ 541.2769; Found 541.2764.
Example 45. tert-Butyl (5-(4,4,5,5-tetramethyl-1,3,2-dioxaborolan-2-yl)pyridin-2-yl) carbamate

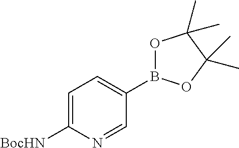 [SMILES: CC(C)(C)OC(=O)Nc1ccc(B2OC(C)(C)C(C)(C)O2)cn1]

To a solution of 5-(4,4,5,5-tetramethyl-1,3,2-dioxaborolan-2-yl)pyridin-2-amine (220 mg, 1.00 mmol), triethylamine (348 μL, 2.50 mmol), and DMAP (12 mg, 0.10 mmol) in DCM (5 mL) was added di-tert-butyl decarbonate (276 μL, 1.20 mmol) at 0° C. The reaction mixture was allowed to warm to RT overnight. The reaction mixture was then diluted with EtOAc and washed with brine, dried over Na₂SO₄, filtered, and concentrated. The residue was purified with flash chromatography using a gradient of EtOAc in hexanes to afford the title compound as white solid. Yield: 147 mg (0.44 mmol, 44%).
¹H NMR (CDCl₃, 600 MHz) δ 9.55 (br, 1H), 8.70 (s, 1H), 8.03 (m, 2H), 1.57 (s, 9H), 1.33 (s, 12H).
Example 46. Ethyl 1-(6-((tert-butoxycarbonyl)amino)pyridin-3-yl)-3,5-dimethyl-1H-pyrazole-4-carboxylate

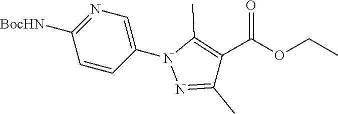 [SMILES: CCOC(=O)c1c(C)nn(-c2ccc(NC(=O)OC(C)(C)C)nc2)c1C]

tert-Butyl (5-(4,4,5,5-tetramethyl-1,3,2-dioxaborolan-2-yl)pyridin-2-yl) carbamate (67 mg, 0.20 mmol) and ethyl 3,5-dimethyl-1H-pyrazole-4-carboxylate (34 mg, 0.20 mmol) were weighed in a scintillation vial and suspended in DMF (2 mL) under ambient atmosphere. To this Cu(OAc)₂ (27 mg, 0.15 mmol) and pyridine (32 μL, 0.40 mmol) were added. The reaction mixture was stirred at 50° C. for 24 h and then diluted with 50% EtOAc in hexanes and washed with brine. Flash chromatography using a gradient of EtOAc in hexanes afforded the title compound. Yield: 37 mg (0.10 mmol, 51%).
¹H NMR (CDCl₃, 600 MHz) δ 8.33 (s, 1H), 8.11 (d, 1H, J=8.9 Hz), 7.97 (br, 1H), 7.70 (dd, 1H, J=8.8, 2.3 Hz), 4.33 (q, 2H, J=7.1 Hz), 2.50 (s, 3H), 2.48 (s, 3H), 1.54 (s, 9H), 1.38 (t, 3H, J=7.1 Hz).
Example 47. Diethyl (1-(6-((tert-butoxycarbonyl)amino)pyridin-3-yl)-3,5-dimethyl-1H-pyrazole-4-carbonyl)-L-valyl-D-glutamate (TT027)

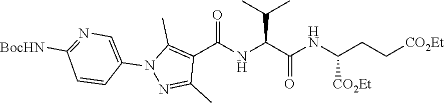 [SMILES: CCOC(=O)CC[C@@H](NC(=O)[C@@H](NC(=O)c1c(C)nn(-c2ccc(NC(=O)OC(C)(C)C)nc2)c1C)C(C)C)C(=O)OCC]

Ethyl 1-(6-((tert-butoxycarbonyl)amino)pyridin-3-yl)-3,5-dimethyl-1H-pyrazole-4-carboxylate (37 mg, 0.10 mmol) was hydrolysed according to the general procedure for alkaline hydrolysis [Intermediate yield: 33 mg (0.10 mmol, quant.)]. This material was used without further purification to synthesise TT027 according to the general procedure for HATU-mediated coupling. Yield: 16 mg (0.026 mmol, 26%).
¹H NMR (CDCl₃, 600 MHz) δ 8.29 (s, 1H), 8.20 (br, 1H), 7.80 (br, 1H), 6.75 (d, 1H, J=7.3 Hz), 6.31 (d, 1H, J=8.1 Hz), 4.58 (m, 2H), 4.20 (m, 2H), 4.13 (q, 2H, J=6.8 Hz), 2.52 (s, 3H), 2.50 (s, 3H), 2.41 (m, 2H), 2.25 (m, 2H), 2.05 (m, 1H), 1.55 (s, 9H), 1.26 (m, 6H), 1.05 (d, 3H, J=6.8 Hz), 1.02 (d, 3H, J=6.8 Hz).
¹³C NMR (CDCl₃, 150 MHz) δ 172.8, 171.4, 171.2, 164.2, 151.9, 151.3, 148.2, 142.8, 136.3, 130.7, 115.0, 112.7, 82.1, 61.8, 60.8, 58.0, 52.0, 31.4, 30.3, 28.2, 27.1, 19.5, 17.9, 14.2, 14.1, 12.1.
HRMS m/z: [M+H]⁺ Calcd for C₃₀H₄₄N₆O₈ 617.3293; Found 617.3290.
Example 48. Diethyl (1-(6-aminopyridin-3-yl)-3,5-dimethyl-1H-pyrazole-4-carbonyl)-L-valyl-D-glutamate (TT026)

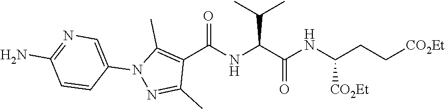 [SMILES: CCOC(=O)CC[C@@H](NC(=O)[C@@H](NC(=O)c1c(C)nn(-c2ccc(N)nc2)c1C)C(C)C)C(=O)OCC]

TT027 (7 mg, 0.011 mmol) was stirred in 4 N HCl in 1,4-dioxane at RT for 4 h. Upon completion of the reaction, the reaction mixture was concentrated to dryness to afford TT026 as HCl salt. Yield: 6 mg (0.011 mmol, quant.).
HRMS m/z: [M+H]⁺ Calcd for C₂₅H₃₆N₆O₆ 517.2769; Found 517.2768.
Example 49. Diethyl N-(1-(4-fluorophenyl)-3-methyl-1H-pyrazole-4-carbonyl)-N-methyl-L-valyl-D-glutamate (TT021)

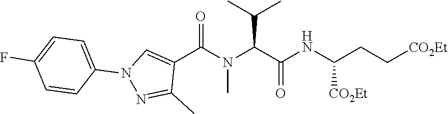 [SMILES: CCOC(=O)CC[C@@H](NC(=O)[C@H](C(C)C)N(C)C(=O)c1cn(-c2ccc(F)cc2)nc1C)C(=O)OCC]

TT021 was synthesised according to the general procedure for HATU-mediated coupling. Yield: 19 mg (0.037 mmol, 37%).
¹H NMR (CDCl₃, 600 MHz) δ 7.98 (s, 1H), 7.61 (m, 2H), 7.14 (m, 3H), 4.66 (d, 1H, J=11.1 Hz), 4.53 (m, 1H), 4.14 (m, 4H), 2.99 (s, 3H), 2.43 (s, 3H), 2.38 (m, 1H), 2.23 (m, 1H), 1.99 (m, 1H), 1.25 (m, 6H), 1.03 (d, 3H, J=6.2 Hz), 0.97 (d, 3H, J=6.5 Hz).
¹³C NMR (CDCl₃, 150 MHz) δ 172.4 (s), 171.6 (s), 170.2 (s), 167.6 (s), 161.4 (d, ¹J_{C,F}=245 Hz), 150.0 (s), 135.9 (s), 128.1 (s), 121.1 (d, ³J_{C,F}=8 Hz), 117.3 (s), 116.3 (d, ²J_{C,F}=23 Hz), 62.9 (s), 61.4 (s), 60.7 (s), 51.5 (s), 33.1 (s), 30.5 (s), 26.9 (s), 25.1 (s), 19.9 (s), 18.6 (s), 14.2 (s), 13.1 (s).
HRMS m/z: [M+H]⁺ Calcd for C₂₆H₃₅FN₄O₆ 519.2613; Found 519.2614.
Example 50. Diethyl N-(1-(4-fluorophenyl)-5-methyl-1H-pyrazole-4-carbonyl)-N-methyl-L-valyl-D-glutamate (TT020)

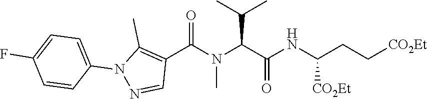 [SMILES: CCOC(=O)CC[C@@H](NC(=O)[C@H](C(C)C)N(C)C(=O)c1cnn(-c2ccc(F)cc2)c1C)C(=O)OCC]

TT020 was synthesised according to the general procedure for HATU-mediated coupling. Yield: 16 mg (0.031 mmol, 31%).
¹H NMR (CDCl₃, 600 MHz) δ 7.71 (s, 1H), 7.41 (m, 2H), 7.19 (m, 3H), 4.63 (d, 1H, J=11.2 Hz), 4.54 (m, 1H), 4.14 (m, 4H), 3.07 (s, 3H), 2.44 (s, 3H), 2.40 (m, 3H), 2.22 (m, 1H), 1.97 (m, 1H), 1.25 (m, 6H), 1.03 (d, 3H, J=6.3 Hz) 0.97 (d, 3H, J=6.6 Hz).
¹³C NMR (CDCl₃, 150 MHz) δ 172.5 (s), 171.6 (s), 170.3 (s), 167.7 (s), 162.3 (d, ¹J_{C,F}=247 Hz), 142.1 (s), 139.3 (s), 135.1 (s), 127.3 (d, ³J_{C,F}=8.7 Hz), 116.2 (d, ²J_{C,F}=23 Hz), 115.6 (s), 62.9 (s), 61.4 (s), 60.7 (s), 51.4 (s), 33.3 (s), 30.5 (s), 27.0 (s), 25.2 (s), 19.9 (s), 18.7 (s), 14.2 (s), 14.1 (s), 11.7 (s).
HRMS m/z: [M+H]⁺ Calcd for C₂₆H₃₅FN₄O₆ 519.2613; Found 519.2613.
Example 51. Diethyl N-(3,5-dimethyl-1-phenyl-1H-pyrazole-4-carbonyl)-N-methyl-L-valyl-D-glutamate (TT019)

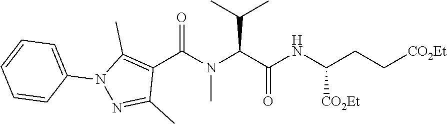 [SMILES: CCOC(=O)CC[C@@H](NC(=O)[C@H](C(C)C)N(C)C(=O)c1c(C)nn(-c2ccccc2)c1C)C(=O)OCC]

To a solution of 3,5-dimethyl-1-phenyl-1H-pyrazole-4-carboxylic acid (22 mg, 0.10 mmol) and DMF (2 drops) in DCM (1.0 mL) was added dropwise oxalyl chloride (43 μL, 0.50 mmol) at 0° C. The reaction mixture was stirred for 1 h and concentrated under vacuum to remove excess oxalyl chloride. This material was used without further purification. To a solution of the dipeptide (0.080 mmol) and DIPEA (70 μL, 0.40 mmol) in DCM (0.5 mL) was added dropwise the acyl chloride in DCM (0.5 mL) at 0° C. The reaction mixture was allowed to warm to RT over 2 days. Upon completion of the reaction, the reaction mixture was directly loaded on column and purified with flash chromatography using a gradient of EtOAc in hexanes. Yield: 16 mg (0.040 mmol, 40% over two steps).
¹H NMR (CDCl₃, 600 MHz) δ 7.44 (m, 5H), 7.24 (br, 1H), 4.67 (d, 1H, J=11.3 hz), 4.52 (m, 1H), 4.14 (m, 4H), 2.93 (s, 3H), 2.42 (m, 3H), 2.33 (m, 6H), 2.22 (m, 1H), 1.98 (m, 1H), 1.25 (m, 6H), 1.04 (d, 3H, J=6.4 Hz), 0.98 (d, 3H, J=6.7 Hz).
¹³C NMR (CDCl₃, 150 MHz) δ 172.4, 171.6, 170.2, 169.1, 139.0, 129.2, 128.1, 125.2, 115.7, 62.7, 61.4, 60.7, 51.6, 32.5, 30.6, 26.9, 24.9, 20.0, 18.7, 14.19, 14.16, 12.9, 11.6.
HRMS m/z: [M+H]⁺ Calcd for C₂₇H₃₈N₄O₆ 515.2864; Found 515.2859.
Example 52. Diethyl (tert-butoxycarbonyl)-D-valyl-L-glutamate

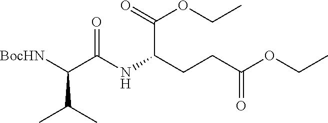 [SMILES: CCOC(=O)CC[C@H](NC(=O)[C@H](NC(=O)OC(C)(C)C)C(C)C)C(=O)OCC]

To a solution of Boc-D-valine-OH (227 mg, 1.04 mmol), diethyl L-glutamate hydrochloride (250 mg, 1.04 mmol), EDC-HCl (200 mg, 1.04 mmol) and HOBt-xH₂O (155 mg, 1.15 mmol) in DMF (5 mL) was added TEA (291 μL, 2.09 mmol) at 0° C. The reaction mixture was allowed to warm to RT overnight. The reaction mixture was quenched with aqueous NH₄Cl and extracted three times with 50% EtOAc in hexanes. Combined organic layers was washed with brine, dried over Na₂SO₄, filtered, concentrated, dried under vacuum for three days. This material was used in proceeding reactions without further purification. Yield: 416 mg (1.03 mmol, 99%), white powder.
¹H NMR (CDCl₃, 600 MHz) δ 6.69 (d, 1H, J=7.0 Hz), 4.97 (m, 1H), 4.58 (dd, 1H, J=12.8, 7.5 Hz), 4.20 (q, 2H, J=7.1 Hz), 4.13 (q, 2H, J=7.1 Hz), 4.00 (m 1H), 2.38 (m, 2H), 2.21 (m, 2H), 2.01 (m, 1H), 1.45 (s, 9H), 1.28 (t, 3H, J=7.1 Hz), 1.25 (t, 3H, J=7.1 Hz), 0.98 (d, 3H, J=6.7 Hz), 0.91 (d, 3H, J=6.8 Hz).
Example 53. Diethyl (1-(4-fluorophenyl)-3,5-dimethyl-1H-pyrazole-4-carbonyl)-D-valyl-L-glutamate (TT007, DL)

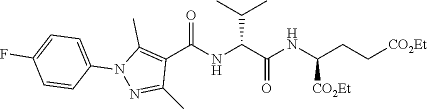 [SMILES: CCOC(=O)CC[C@H](NC(=O)[C@H](NC(=O)c1c(C)nn(-c2ccc(F)cc2)c1C)C(C)C)C(=O)OCC]

TT007, DL was synthesised according to the general procedure for HATU-mediated coupling. The product was purified with column chromatography eluting with 75% EtOAc in hexanes. Yield: 28 mg (0.054 mmol, 54%).
¹H NMR (CDCl₃, 600 MHz) δ 7.36 (dd, 2H, J_{H,H}=8.8 Hz, ⁴J_{H,F}=4.7 Hz), 7.17 (dd, 2H, J_{H,H}=8.5 Hz, ³J_{H,F}=8.5 Hz), 6.74 (br, 1H), 6.31 (d, 1H, J=8.1 Hz), 4.58 (m, 2H), 4.20 (m, 2H), 4.12 (m, 2H), 2.53 (s, 3H), 2.48 (s, 3H), 2.40 (m, 2H), 2.24 (m, 2H), 2.05 (m, 1H), 1.26 (m, 6H), 1.06 (d, 3H, J=6.7 Hz), 1.02 (d, 3H, J=6.8 Hz).
Example 54. Diethyl (1-(2-methoxyphenyl)-3,5-dimethyl-1H-pyrazole-4-carbonyl)-D-valyl-L-glutamate (TT035, DL)

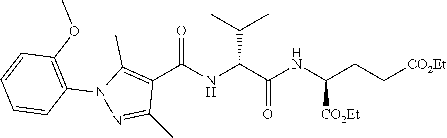 [SMILES: CCOC(=O)CC[C@H](NC(=O)[C@H](NC(=O)c1c(C)nn(-c2ccccc2OC)c1C)C(C)C)C(=O)OCC]

TT035, DL was synthesised according to the general procedure for HATU-mediated coupling. The product was purified with column chromatography eluting with 75% EtOAc in hexanes. Yield: 32 mg (0.060 mmol, 60%).
¹H NMR (CDCl₃, 600 MHz) δ 7.43 (ddd, 1H, J=7.9, 7.9, 1.4 Hz), 7.29 (dd, 1H, J=7.7, 1.4 Hz), 7.05 (dd, 1H, J=7.7, 7.7 Hz), 7.02 (d, 1H, J=8.3 Hz), 6.74 (d, 1H, J=7.4 Hz), 6.28 (d, 1H, J=8.1 Hz), 4.58 (m, 2H), 4.20 (m, 2H), 4.12 (m, 2H), 3.80 (s, 3H), 2.54 (s, 3H), 2.41 (m, 2H), 2.32 (s, 3H), 2.25 (m, 2H), 2.05 (m, 1H), 1.26 (m, 6H), 1.06 (d, 3H, J=6.8 Hz), 1.03 (d, 3H, J=6.8 Hz).
Example 55. Diethyl (1-(1H-indol-6-yl)-3,5-dimethyl-1H-pyrazole-4-carbonyl)-D-valyl-L-glutamate (TT025, DL)

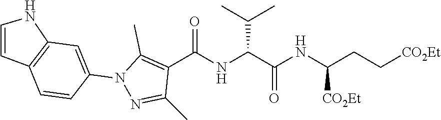 [SMILES: CCOC(=O)CC[C@H](NC(=O)[C@H](NC(=O)c1c(C)nn(-c2ccc3cc[nH]c3c2)c1C)C(C)C)C(=O)OCC]

TT025, DL was synthesised according to the general procedure for HATU-mediated coupling. The product was purified with column chromatography eluting with 75% EtOAc in hexanes. Yield: 20 mg (0.037 mmol, 34%).
¹H NMR (CDCl₃+CD₃OD, 600 MHz) δ 10.03 (br, 1H), 7.89 (s, 1H), 7.79 (d, 1H, J=7.5 Hz), 7.60 (d, 1H, J=8.3 Hz), 7.31 (s, 1H), 6.94 (d, 1H, J=8.3 Hz), 6.62 (d, 1H, J=8.5 Hz), 6.49 (s, 1H), 4.42 (m, 2H), 4.11 (q, 2H, J=7.1 Hz), 4.05 (q, 2H, J=6.8 Hz), 2.43 (s, 3H), 2.36 (s, 5H), 2.13 (m, 2H), 1.95 (m, 1H), 1.19 (m, 6H), 0.97 (d, 3H, J=6.7 Hz), 0.94 (d, 3H, J=6.7 Hz).
Example 56. Diethyl (3,5-dimethyl-1-(2-oxo-1,2-dihydropyridin-4-yl)-1H-pyrazole-4-carbonyl)-D-valyl-L-glutamate (TT030, DL)

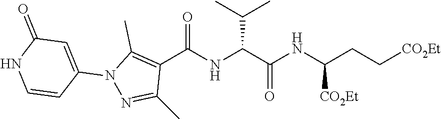 [SMILES: CCOC(=O)CC[C@H](NC(=O)[C@H](NC(=O)c1c(C)nn(-c2cc[nH]c(=O)c2)c1C)C(C)C)C(=O)OCC]

TT030, DL was synthesised according to the general procedure for HATU-mediated coupling. Upon completion of the reaction, reaction mixture was diluted with EtOAc, washed with aqueous NH₄Cl, dried over Na₂SO₄, filtered, and concentrated. The residue was purified with column chromatography using two different columns and eluents: first with 5% MeOH/DCM; then on a new column with 100% THF. Yield: 9 mg (0.017 mmol, 16%).
¹H NMR (CDCl₃, 600 MHz) δ 7.41 (d, 1H, J=7.1 Hz), 6.94 (br, 1H), 6.63 (m, 2H), 6.51 (d, 1H, J=7.1 Hz), 4.57 (m, 2H), 4.20 (m, 2H), 4.13 (q, 2H, J=7.1 Hz), 2.62 (s, 3H), 2.48 (s, 3H), 2.41 (m, 2H), 2.25 (m, 2H), 2.07 (m, 1H), 1.27 (m, 6H), 1.06 (d, 3H, J=6.8 Hz), 1.03 (d, 3H, J=6.8 Hz).
Example 57. Diethyl (tert-butoxycarbonyl)glycyl-L-valyl-D-glutamate

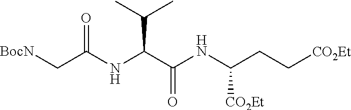

Diethyl (tert-butoxycarbonyl)-L-valyl-D-glutamate (150 mg, 0.37 mmol) was deprotected with 25% TFA/DCM (4 mL). To a solution of Boc-Gly-OH (72 mg, 0.41 mmol), HBTU (79 mg, 0.41 mmol), and TEA (260 μL, 1.86 mmol) in DMF (4 mL) was added the deprotected amine at 0° C. The reaction mixture was allowed to warm to RT over 1 h, diluted with EtOAc, washed with aqueous NaHCO₃, aqueous NH₄Cl, and brine, dried over MgSO₄, filtered, and concentrated. The residue was purified with column chromatography (25% EtOAc/DCM). Yield: 59 mg (0.13 mmol, 34%), yellow oil.
¹H NMR (CDCl₃, 600 MHz) δ 7.00 (br, 1H), 6.63 (br, 1H), 5.23 (br, 1H), 4.53 (m, 1H), 4.38 (m, 1H), 4.21 (q, 2H, J=7.0 Hz), 4.16 (q, 2H, J=7.0 Hz), 3.91 (m, 1H), 3.82 (m, 1H), 2.5-2.0 (m, 5H), 1.49 (s, 9H), 1.29 (m, 6H), 0.99 (d, 3H, J=6.7 Hz), 0.95 (d, 3H, J=6.8 Hz).
Example 58. Diethyl cinnamoylglycyl-L-valyl-D-glutamate [CinGVE]

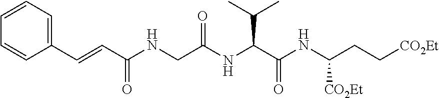 [SMILES: CCOC(=O)CC[C@@H](NC(=O)[C@@H](NC(=O)CNC(=O)/C=C/c1ccccc1)C(C)C)C(=O)OCC]

Diethyl (tert-butoxycarbonyl)glycyl-L-valyl-D-glutamate (30 mg, 0.065 mmol) was deprotected with 25% TFA/DCM (1.5 mL). To the solution of the deprotected amine and TEA (45 μL, 0.33 mmol) in DMF (0.6 mL) was added cinnamoyl chloride (22 mg, 0.13 mmol) at 0° C. The reaction mixture was allowed to warm to RT over 30 min, diluted with EtOAc, washed with aqueous NaHCO₃, aqueous NH₄Cl, and brine, dried over MgSO₄, filtered, and concentrated. The residue was purified with flash chromatography using a gradient of EtOAC in DCM. Yield: 16 mg (33 μmol, 50%), white solid.
¹H NMR (CDCl₃, 600 MHz) δ 7.69 (d, 1H, J=15.6 Hz), 7.54 (m, 2H), 7.40 (m, 3H), 6.98 (br, 1H), 6.66 (br, 1H), 6.52 (br, 1H), 6.51 (d, 1H, J=15.7 Hz), 4.57 (m, 1H), 4.41 (m, 1H), 4.17 (m, 1H), 2.5-2.0 (m, 5H), 1.27 (m, 6H), 1.00 (d, 3H, J=6.7 Hz), 9.65 (d, 3H, J=6.8 Hz).
HRMS m/z: [M+H]⁺ Calcd for C₂₅H₃₆N₃O₇ 490.2548; Found 490.2559.

Preparation of NOD2/LRR-MDP docking model. All computational studies were carried out using the Schrödinger Drug Discovery Platform and RDKit. Structures and NOD2 agonist activity data of MDP and its analogues and derivatives were curated from literature. These ligands were prepared for docking using LigPrep to test the performance of docking models. MDP conformers were generated using an expanded sampling protocol in MacroModel ¹. The leucine-rich repeat (LRR) domain of NOD2 was excised from the crystal structure of OcNOD2 (PDB ID: 5IRN) and prepared for docking using Protein Preparation Wizard. A receptor-based pharmacophore model was generated by E-Pharmacophore. In Phase, this pharmacophore model was used to screen the MDP conformers to generate initial docking models. These docking models were then refined using Glide SP with vdW scaling and clustered, and 100 representative models were selected. For each of the 100 models, the side chains within 5 Å of MDP were minimised using Prime MM-GB/SA to account for local receptor flexibility. Resulting receptors were clustered, and 13 representative models were selected for re-docking. In each receptor of the ensemble was docked MDP using Glide SP-PEP. These docking models were then refined and rescored using Prime MM-GB/SA, and 10 top-scoring models were selected for further evaluation. To test if any of these models can correctly tell active ligands from inactive ligands, the set of known MDP analogues and derivatives was docked using Glide SP while their maximum common structures with MDP were fixed in position. For desmuramyl dipeptides with aromatic surrogates, an additional positional constraint for an aromatic atom was inferred from receptor-based pharmacophore models and applied in Glide XP. The best-performing model was selected based on the balance of sensitivity and selectivity and visually inspected.

DMDP library preparation and screening. Chemical structures of Enamine Carboxylic Acid Building Blocks were downloaded as SDF (07/18/2020). Using an RDKit script, these carboxylic acid derivatives (31344 compounds) were conjugated with L-Val-D-Glu, and resulting DMDPs were filtered against PAINS and required to have at least one aromatic ring and to reside within the beyond-rule-of-five space boundaries. The filtered ligands were prepared for docking using LigPrep and docked using a cascade of Glide SP (passed: 3938 compounds) and XP (passed: 1683 compounds) while their maximum common structures with MDP were fixed in position. Resulting models were refined and rescored using Prime MM-GB/SA, and the top 10% (168 ligands) were selected for further analysis. Filtering out ligands that considerably deviated from their original docking poses during the refinement (RMSD for MCS with MDP>1.5 Å) and tautomers left 77 unique compounds, which were clustered and visually inspected.

Reporter cell line activation assay. Approximately 24 h before the assay, culture medium for each of the HEK-Blue reporter cell lines was replaced with a 1:1 mixture of DMEM supplemented with 10% FBS and OptiMEM. On the day of the assay, single cell suspensions (150,000 cells/mL) were produced in HEK-Blue detection medium (InvivoGen, hb-det2). Cells were aliquoted at 200 μL per well of a 96-well plate containing 0.2 μL of a 1000× stock of each test compound in DMSO. Cells were then incubated at 37° C. in 5% CO₂ for 8-10 h. To measure activity, wells were then gently pipetted up and down to mix the conditioned medium, and absorbance from the colorimetric product of the secreted alkaline phosphatase was measured at 630 nm. In some assays, cells were stimulated for 16 h with compounds as described above in complete DMEM. Then, 20 μL of the conditioned medium was mixed with 180 μL QUANTI-Blue solution (InvivoGen, rep-qbs) and incubated for 2-4 h at 37° C. Activity was then measured by absorbance as described above.

Cytokine release assay. Human PMBCs were obtained from whole blood donated by healthy individuals via the Scripps Normal Blood Donor Service. Whole blood was diluted 1:1 with phosphate-buffered saline (PBS) containing 2% FBS and then layered onto Lymphoprep (STEMCELL Technologies, 07851) in SepMate-50 isolation tubes (STEMCELL Technologies, 85480). The discontinuous gradient was centrifuged for 10 min at 1,200×g, and mononuclear cells were removed by pouring off. Mononuclear cells were then resuspended in PBS+2% FBS and centrifuged for 3 min at 200×g twice before further use. Washed mononuclear cells were then aliquoted at 5×10⁵ cells per well of a 48-well plate with 300 μL RPMI containing 10% FBS (complete RPMI). Cells were then diluted with 100 μL complete RPMI containing 50 μM of the test compound (10 μM final concentration). Then, 100 μL complete RPMI (-LPS) or 100 μL complete RPMI containing 5 ng mL-1 LPS (+LPS, 10 ng mL⁻¹ final concentration) was added. Cells were incubated overnight at 37° C. in 5% CO₂, and conditioned medium was harvested and centrifuged for 5 min at 500×g to remove residual cells. Samples were used directly for cytokine analysis with the LEGENDPlex Human Inflammatory Panel 1 (BioLegend, 740809) according to the manufacturer's protocol. For analysis, 25 μL of the conditioned medium was used for −LPS samples, and 12.5 μL was used for the +LPS samples. On-bead ELISA readouts were measured using the standard PE and APC detection filter parameters on a ZE5 flow cytometer (Bio-Rad; 405, 488, 561, and 640 lasers) in the Scripps Research Flow Cytometry Core Facility. Raw data files were then imported into the LEGENDPlex Cloud Data Analysis software suite (BioLegend) and analyzed using the manufacturer's pre-set parameters.

Tumor growth assay. B16-F10 cells were subcultured on the day prior to harvesting for tumor inoculation. On the day of injection, cells were harvested using TrypLE Express, washed and resuspended in cold PBS, and counted with a hemocytometer. Cells were then resuspended at 2×10⁶ cells mL⁻¹ in PBS and mixed 1:1 with Matrigel matrix (Corning; growth factor reduced, 356231). Cell suspensions were kept on ice prior to injection. Animals were anesthetized using 3% isoflurane, and their right flank was shaved with hair clippers. Animals were then subcutaneously injected with 100 μL of the cell suspension on their mid-right flank (1×10⁵ cells per injection). Once tumors were established at roughly 25-100 mm³, tumor volume was measured every other day. Digital calipers were used to measure the length and width of each tumor, and tumor volume was calculated as length×width×0.5, where width was the smaller of the two measurements. Checkpoint inhibitor treatment was started two days following the first measurement and continued every other day for three total injections. For each injection, 100 μg anti-PD-L1 (BioXCell, BP0101) and 100 μg of either MDP (InvivoGen, tlrl-mdp), TT030, or TT030-ent were intraperitoneally injected using 200 μL antibody diluent (BioXCell, IP0065). Animals were humanely euthanized with CO₂ asphyxiation at the end of each experiment.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A compound of Formula I 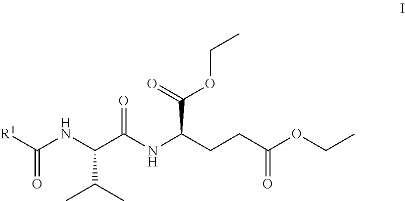
wherein:
R¹ is —(C₁-C₁₀)alkyl, —(C₁-C₁₀)heteroalkyl, —(C₂-C₁₀)alkenyl, —(C₂-C₁₀)alkynyl, —(C₂-C₁₀)heteroalkenyl, —(C₃-C₇)cycloalkyl, —(C₁-C₆)alkyl(C₃-C₇)cycloalkyl, —(C₃-C₇)cycloalkenyl, —(C₁-C₆)alkyl(C₃-C₇)cycloalkenyl, —(C₃-C₇)heterocycloalkyl, —(C₃-C₇)heterocycloalkenyl, —(C₁-C₆)alkyl(C₃-C₇)heterocycloalkyl, —(C₁-C₆)alkyl(C₃-C₇)heterocycloalkenyl, —(C₆-C₁₀)aryl, —(C₁-C₆)alkyl(C₆-C₁₀)aryl, —(C₅-C₁₀)heteroaryl, and —(C₁-C₆)alkyl(C₅-C₁₀)heteroaryl, wherein each is optionally and independently substituted with one or more R^{1′};
each R^{1′} is independently R^{1a} or R^{1b},
R^{1a} is H, halo, —CN, —OH, —NH₂, or —NHC(═O)O(C₁-C₆)alkyl;
R^{1b} is —(C₁-C₁₀)alkyl, —(C₁-C₁₀)heteroalkyl, —(C₂-C₁₀)alkenyl, —(C₂-C₁₀)alkynyl, —(C₂-C₁₀)heteroalkenyl, —(C₃-C₇)cycloalkyl, —(C₁-C₆)alkyl(C₃-C₇)cycloalkyl, —(C₃-C₇)cycloalkenyl, —(C₁-C₆)alkyl(C₃-C₇)cycloalkenyl, —(C₃-C₇)heterocycloalkyl, —(C₃-C₇)heterocycloalkenyl, —(C₁-C₆)alkyl(C₃-C₇)heterocycloalkyl, —(C₁-C₆)alkyl(C₃-C₇)heterocycloalkenyl, —(C₆-C₁₀)aryl, —(C₁-C₆)alkyl(C₆-C₁₀)aryl, —(C₅-C₁₀)heteroaryl, or —(C₁-C₆)alkyl(C₅-C₁₀)heteroaryl, wherein each is optionally and independently substituted with one or more R^{1″}; and
each R^{1″} is independently H, halo, oxo, —CN, —OH, —(C₁-C₆)alkyl, —O(C₁-C₆)alkyl-NH₂, —NHC(═O)O(C₁-C₆)alkyl, —(C₁-C₆)heteroalkyl, —(C₁-C₆) haloalkyl, —(C₂-C₆)alkenyl, —(C₂-C₆)alkynyl, —(C₃-C₇)cycloalkyl, —(C₃-C₇)cycloalkenyl, —(C₃-C₇)heterocycloalkyl, or —(C₃-C₇)heterocycloalkenyl;
with the proviso that Formula I is not diethyl ((E)-3-(4-hydroxy-3-methoxyphenyl)acryloyl)glycyl-L-valyl-D-glutamate; diethyl (tert-butoxycarbonyl)glycyl-L-valyl-D-glutamate; diethyl ((E)-3-(4-((tert-butoxycarbonyl)amino)phenyl)acryloyl)glycyl-L-valyl-D-glutamate; diethyl ((E)-3-(4-aminophenyl)acryloyl)glycyl-L-valyl-D-glutamate; diethyl(2-(3,4-difluorophenyl)cyclopropane-1-carbonyl)glycyl-L-valyl-D-glutamate; diethyl (6-phenyl-1H-indole-2-carbonyl)glycyl-L-valyl-D-glutamate; diethyl cinnamoylglycyl-L-valyl-D-glutamate; diethyl(2-phenylcyclopropane-1-carbonyl)glycyl-L-valyl-D-glutamate; or diethyl ((E)-3-(3,4-difluorophenyl)acryloyl)glycyl-L-valyl-D-glutamate;
or a pharmaceutically acceptable salt thereof.

2. The compound of claim 1, wherein R¹ is —(C₁-C₁₀)alkyl optionally substituted with one or more R^{1′}.

3. The compound of claim 1, wherein R¹ is —(C₂-C₁₀)alkenyl optionally substituted with one or more R^{1′}.

4. The compound of claim 1, wherein R¹ is —(C₃-C₇)cycloalkyl optionally substituted with one or more R^{1′}.

5. The compound of claim 4, wherein —(C₃-C₇)cycloalkyl is cyclopropyl.

6. The compound of claim 4, wherein —(C₃-C₇)cycloalkyl is cyclopentyl.

7. The compound of claim 1, wherein R¹ is —(C₁-C₆)alkyl(C₃-C₇)heterocycloalkenyl optionally substituted with one or more R^{1′}.

8. The compound of claim 7, wherein —(C₁-C₆)alkyl(C₃-C₇)heterocycloalkenyl is methylene diazirinyl.

9. The compound of claim 1, wherein R¹ is —(C₅-C₁₀)heteroaryl optionally substituted with one or more R^{1′}.

10. The compound of claim 9, wherein —(C₅-C₁₀)heteroaryl is pyrazolyl.

11. The compound of claim 10, wherein R¹ is pyrazolyl optionally substituted with one or more —(C₁-C₆)alkyl and further substituted with one or more R^{1′}.

12. The compound of claim 11, wherein R¹ is pyrazolyl substituted with one or more —CH₃ and further substituted with one or more R^{1′}.

13. The compound of claim 12, wherein R¹ is pyrazolyl substituted with one or more —CH₃ and further substituted with one or more R^{1′}.

14. The compound of claim 13, wherein R¹ is methyl pyrazolyl further substituted with one or more R^{1′}.

15. The compound of claim 13, wherein R¹ is dimethyl pyrazolyl further substituted with one or more R^{1′}.

16. The compound of claim 1, having the structure of Formula II

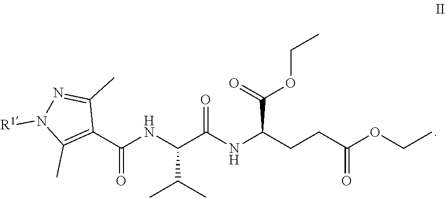

17. The compound of claim 1, having the structure of Formula III 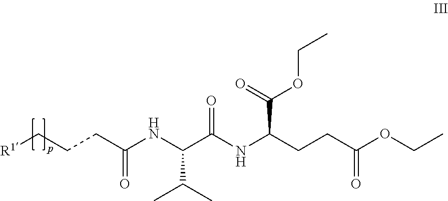
wherein: [symbol]
represents a single or double bond; and
n is 0 to 6.

18. The compound of claim 17, wherein n is 0.

19. The compound of claim 17, wherein n is 1.

20. The compound of claim 17, wherein n is 2.

21. The compound of any one of claims 17-20, wherein [symbol] is a single bond.

22. The compound of any one of claims 17-20, wherein [symbol] is a double bond.

23. The compound of claim 1, having the structure of Formula IV

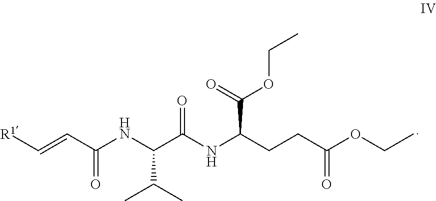

24. The compound of claim 1, having the structure of Formula V 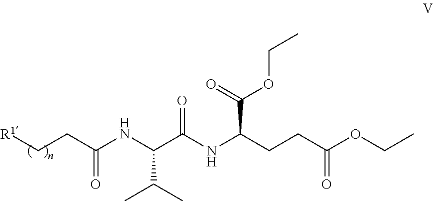
wherein:
n is 0 to 3.

25. The compound of claim 24, wherein n is 0.

26. The compound of claim 24, wherein n is 1.

27. The compound of claim 24, wherein n is 2.

28. The compound of claim 1, having the structure of Formula VI 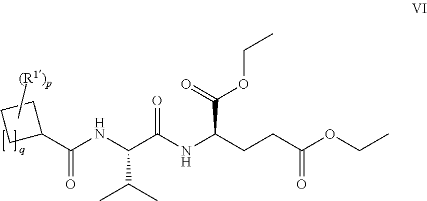
wherein:
p is 0 to 3; and
q is 0 to 3.

29. The compound of claim 28, wherein q is 0 and p is 1.

30. The compound of claim 28, wherein q is 1 and p is 1.

31. The compound of claim 28, wherein q is 2 and p is 1.

32. The compound of any one of claims 1-31, wherein R^{1′} is Ph optionally substituted with one or more R^{1″}.

33. The compound of any one of claims 1-31, wherein R^{1′} is —(C₅-C₁₀)heteroaryl optionally substituted with one or more R^{1″}.
The compound of claim 33, wherein R^{1′} is pyridinyl optionally substituted with one or more R^{1″}.

34. The compound of claim 33, wherein R^{1′} is 1,2-dihydropyridinyl optionally substituted with one or more R^{1″}.

35. The compound of claim 33, wherein R^{1′} is pyrimidinyl optionally substituted with one or more R^{1″}.

36. The compound of claim 33, wherein R^{1′} is 1-H-indolyl optionally substituted with one or more R^{1″}.

37. The compound of claim 33, wherein R^{1′} is 1-H-pyrrolo[2,3-b]pyridinyl optionally substituted with one or more R^{1″}.

38. The compound of any one of claims 1-39, wherein one R^{1″} is present.

39. The compound of claim 39, wherein R^{1″} is halo.

40. The compound of claim 40, wherein halo is F.

41. The compound of claim 40, wherein halo is Cl.

42. The compound of claim 39, wherein R^{1″} is —OMe.

43. The compound of claim 39, wherein R^{1″} is —NH₂.

44. The compound of claim 35, wherein R^{1″} is oxo.

45. The compound of claim 39, wherein R^{1″} is oxo.

46. The compound of claim 39, wherein R^{1″} is Me.

47. The compound of claim 39, wherein R^{1″} is —OH.

48. The compound of any one of claims 1-38, wherein two R^{1″} are present.

49. The compound of claim 49, wherein at least one R^{1″} is halo.

50. The compound of claim 49, wherein both R^{1″} are halo.

51. The compound of either claim 49 or claim 50, wherein halo is F.

52. The compound of either claim 49 or claim 50, wherein halo is Cl.

53. A compound having any one of the formulae selected from the group consisting of:
diethyl (4-phenylbutanoyl)-L-valyl-D-glutamate;
diethyl ((E)-3-(4-fluorophenyl)acryloyl)-L-valyl-D-glutamate;
diethyl (1-(4-fluorobenzyl)cyclopentane-1-carbonyl)-L-valyl-D-glutamate;
rac-diethyl ((1R,2R)-2-(3,4-dichlorophenyl)cyclopropane-1-carbonyl)-L-valyl-D-glutamate;
rac-diethyl ((R)-3-(3,4-difluorophenyl)-2-methylpropanoyl)-L-valyl-D-glutamate;
diethyl (1-(4-fluorophenyl)-3,5-dimethyl-1H-pyrazole-4-carbonyl)-L-valyl-D-glutamate;
diethyl (1-(4-fluorophenyl)-5-methyl-1H-pyrazole-4-carbonyl)-L-valyl-D-glutamate;
diethyl (1-(4-fluorophenyl)-3-methyl-1H-pyrazole-4-carbonyl)-L-valyl-D-glutamate;
diethyl (1-(4-fluorophenyl)-5-isopropyl-1H-pyrazole-4-carbonyl)-L-valyl-D-glutamate;
diethyl (3-methyl-1-(p-tolyl)-1H-pyrazole-4-carbonyl)-L-valyl-D-glutamate;
diethyl (1-(2-hydroxyphenyl)-5-methyl-1H-pyrazole-4-carbonyl)-L-valyl-D-glutamate;
diethyl (5-methyl-1-(pyridin-4-yl)-1H-pyrazole-4-carbonyl)-L-valyl-D-glutamate;
diethyl (3,5-dimethyl-1-phenyl-1H-pyrazole-4-carbonyl)-L-valyl-D-glutamate;
diethyl (1-(3,4-dichlorophenyl)-3,5-dimethyl-1H-pyrazole-4-carbonyl)-L-valyl-D-glutamate;
diethyl (1-(4-methoxyphenyl)-3,5-dimethyl-1H-pyrazole-4-carbonyl)-L-valyl-D-glutamate;
diethyl (1-(4-chlorophenyl)-3,5-dimethyl-1H-pyrazole-4-carbonyl)-L-valyl-D-glutamate;
diethyl (1-(1H-indol-6-yl)-3,5-dimethyl-1H-pyrazole-4-carbonyl)-L-valyl-D-glutamate;
diethyl (1-(6-aminopyridin-3-yl)-3,5-dimethyl-1H-pyrazole-4-carbonyl)-L-valyl-D-glutamate;
diethyl (1-(6-((tert-butoxycarbonyl)amino)pyridin-3-yl)-3,5-dimethyl-1H-pyrazole-4-carbonyl)-L-valyl-D-glutamate;
diethyl (1-(1H-indol-5-yl)-3,5-dimethyl-1H-pyrazole-4-carbonyl)-L-valyl-D-glutamate;
diethyl (3,5-dimethyl-1-(1H-pyrrolo[2,3-b]pyridin-5-yl)-1H-pyrazole-4-carbonyl)-L-valyl-D-glutamate;
diethyl (3,5-dimethyl-1-(2-oxo-1,2-dihydropyridin-4-yl)-1H-pyrazole-4-carbonyl)-L-valyl-D-glutamate;
diethyl (3,5-dimethyl-1-(pyridin-4-yl)-1H-pyrazole-4-carbonyl)-L-valyl-D-glutamate;
diethyl (3,5-dimethyl-1-(pyridin-2-yl)-1H-pyrazole-4-carbonyl)-L-valyl-D-glutamate;
diethyl (3,5-dimethyl-1-(pyridin-3-yl)-1H-pyrazole-4-carbonyl)-L-valyl-D-glutamate;
diethyl (3,5-dimethyl-1-(pyrimidin-5-yl)-1H-pyrazole-4-carbonyl)-L-valyl-D-glutamate;
diethyl (1-(2-methoxyphenyl)-3,5-dimethyl-1H-pyrazole-4-carbonyl)-L-valyl-D-glutamate;
diethyl (1-(3-methoxyphenyl)-3,5-dimethyl-1H-pyrazole-4-carbonyl)-L-valyl-D-glutamate;
diethyl (1-(pyridin-3-yl)-1H-pyrazole-4-carbonyl)-L-valyl-D-glutamate; and
diethyl(2-(3-(but-3-yn-1-yl)-3H-diazirin-3-yl) acetyl)-L-valyl-D-glutamate.

54. A compound having the formula diethyl (3,5-dimethyl-1-(2-oxo-1,2-dihydropyridin-4-yl)-1H-pyrazole-4-carbonyl)-L-valyl-D-glutamate.

55. A pharmaceutical composition comprising the compound of any one of claims 1-55, admixed with a pharmaceutically acceptable carrier, diluent, or excipient.

56. The pharmaceutical composition of claim 56, further comprising one or more adjuvants, immunotherapeutic agents, or anti-infective compounds or compositions.

57. The pharmaceutical composition of claim 57, wherein the one or more adjuvants, immunotherapeutic agents, or anti-infective compounds or compositions is IL-1β, TNFα, IL-12p70, or anti-PD-L1.

58. The pharmaceutical composition of claim 57, wherein the one or more adjuvants, immunotherapeutic agents, or anti-infective compounds or compositions is an additional NOD2 agonist compound or composition.

59. The pharmaceutical composition of claim 59, wherein the additional NOD2 agonist compound or composition includes, but is not limited to, MDP, dMDP analogues including CinGVE, MurNAc, L18-MDP, N-glycolyl-MDP, murabutide, mifamurtide, M-TriLYS, and CL429.

60. A method of activating NOD2, comprising administering to a subject infected with a NOD2-responsive infection, disease, or disorder a therapeutically effective amount of the compound of any one of claims 1-55 or the pharmaceutical composition of claims 56-60.

61. A method of preventing, ameliorating, or treating a NOD2-responsive infection, disease, or disorder, comprising administering to a subject in need thereof a therapeutically effective amount of the compound of any one of claims 1-55 or the pharmaceutical composition of claims 56-60.

62. The method of claim 62, further comprising administration of one or more additional therapeutic compounds or compositions.

63. The method of claim 63, wherein the one or more therapeutic compounds or compositions is an adjuvant, anti-infective, immunotherapeutic, or anti-cancer therapeutic compound or composition.

64. The method of claim 64, wherein at least one of the one or more therapeutic compounds or compositions is an immunotherapeutic compound or composition.

65. The method of claim 63, wherein at least one of the one or more therapeutic compounds or compositions is an anti-cancer therapeutic compound or composition.

66. Use of the compound of any one of claims 1-55 as an adjuvant in immunotherapy.

67. Use of the compound of any one of claims 1-55 as an anti-infective agent.

68. Use of the compound of any one of claims 1-55 as an anti-cancer agent.

69. The use of any one of claims 67-69, wherein the compound is the compound of claim 55.

70. Any compound, composition, method, or use as described herein.
